(19) United States
(12) Patent Application Publication
(10) Pub. No.: US 2023/0355939 A1
(43) Pub. Date: Nov. 9, 2023

(54) DEVICES AND METHODS FOR CONTINUOUS EXTRUSION OF A DRUG INTO THE MOUTH

(71) Applicant: SynAgile Corporation, San Jose, CA (US)

(72) Inventors: Adam HELLER, Austin, TX (US); John SPIRIDIGLIOZZI, West Roxbury, MA (US); Steven DIIANNI, Groveland, MA (US); Robert DRAPER, Jamaica Plain, MA (US); John HOWARD, Salem, MA (US)

(21) Appl. No.: 18/025,459

(22) PCT Filed: Sep. 15, 2021
(86) PCT No.: PCT/US2021/050555

§ 371 Date: Mar. 9, 2023

Related U.S. Application Data
(60) Provisional application No. 63/078,796, filed on Sep. 15, 2020.

(51) Int. Cl. A61M 31/00; A61M 5/142; A61J 7/00; A61M 39/20

(52) U.S. Cl. CPC A61M 31/002; A61J 7/0092; A61M 5/142; A61M 39/20

(57) ABSTRACT

Devices and methods are disclosed for continuous extrusion of a drug-containing fluid into the mouth of a patient from a device residing in the mouth of the patient. Methods of treating a disease by such extrusion are also disclosed.

### FIELD OF THE INVENTION

The invention features a drug delivery device anchored in the mouth for continuously administering a pharmaceutical composition and methods of using said device.

### BACKGROUND OF THE INVENTION

This invention relates to devices and methods for continuous or semi-continuous drug administration via the oral route. It is an aim of this invention to solve several problems related to drugs with short physiological half-lives of drugs (e.g., shorter than 8 hours, 6 hours, 4 hours, 2 hours, 1 hour, 30 min, 20 min or 10 min) and/or narrow therapeutic windows of drugs that are currently dosed multiple times per day: it is inconvenient to take a drug that must be dosed multiple times per day or at night, the drug’s pharmacokinetics and efficacy may be sub-optimal, and side effects may increase in frequency and/or severity. Continuous or semi-continuous administration can be particularly beneficial for drugs with a short half-life (e.g., in the plasma), and/or short persistence of the drug’s physiological effect, and/or a narrow therapeutic window, such as levodopa (LD), muscle relaxants (e.g., baclofen for managing spasticity), anti-epileptics (e.g., oxcarbazepine, topiramate, lamotrigine, gabapentin, carbamazepine, valproic acid, levetiracetam, pregabalin), parasympathomimetics (e.g., pyridostigmine) and sleep medications (e.g., zaleplon). Continuous or semi-continuous infusion in the mouth can provide for lesser fluctuation in the concentration of a drug in an organ or fluid, for example in the blood or plasma. Convenient, automatic administration of a drug can also increase patient compliance with their drug regimen, particularly for patients who must take medications at night and for patients with dementia.

Medical conditions managed by continuously orally administered drugs include Parkinson’s disease, spasticity, muscular weakness, bacterial infections, viral infections, fungal infections, parasite caused diseases, cancer, pain, organ transplantation, disordered sleep, epilepsy and seizures, anxiety, mood disorders, post-traumatic stress disorder, arrhythmia, hypertension, heart failure, dementia, allergies, and diabetic nephropathy.

Most drugs intended for oral administration have been formulated as solids (e.g., pills, tablets), solutions, or suspensions that are administered once or several times per day. Such drugs are not formulated to meet the requirements of continuous or semi-continuous, constant-rate, intra-oral administration. For example, many suspensions and solutions have been formulated in relatively large daily volumes that don’t fit in the mouth without interfering with its functions, particularly with speech, and/or in formulations that are physically or chemically unstable over the course of a day at body temperature; and pills and tablets have rarely been formulated in units and dosage amounts appropriate for dosing frequently throughout the day.

Large quantities of drug must be administered to treat some diseases. For example, 1,000 mg of levodopa is a typical daily dose administered to patients with advanced Parkinson’s disease. In order to continuously administer such large quantities of drug into the mouth in a fluid volume that will fit comfortably in the mouth (typically less than 5 mL) for many hours, it is sometimes necessary to employ concentrated, often viscous, fluid formulations of the drug. Use of viscous fluids can provide the small volumes, high concentrations, uniform drug dispersion, storage stability, and operational stability desired for the drugs and methods of the invention. Consequently, it is often necessary to employ miniaturized pumps tailored to provide the pressures required to pump the viscous fluids. The drug devices and formulations of the invention address these unmet needs.

As a specific example, Parkinson’s disease (PD) is characterized by the inability of the dopaminergic neurons in the substantia nigra to produce the neurotransmitter dopamine. PD impairs motor skills, cognitive processes, autonomic functions, and sleep. Motor symptoms include tremor, rigidity, slow movement (bradykinesia), and loss of the ability to initiate movement (akinesia) (collectively, the “off” state). Non-motor symptoms of PD include dementia, dysphagia (difficulty swallowing), slurred speech, orthostatic hypotension, seborrheic dermatitis, urinary incontinence, constipation, mood alterations, sexual dysfunction, and sleep issues (e.g., daytime somnolence, insomnia).

After more than 40 years of clinical use levodopa (LD) therapy remains the most effective method for managing PD and provides the greatest improvement in motor function. Consequently, LD administration is the primary treatment for PD. LD is usually orally administered. The orally administered LD enters the blood and part of the LD in the blood crosses the blood brain barrier. It is metabolized, in part, in the brain to dopamine which temporarily diminishes the motor symptoms of PD. As the neurodegeneration underlying PD progresses, the patients require increasing doses of LD and the fluctuations of brain dopamine levels increase. When too much LD is transported to the brain, dyskinesia sets in (uncontrolled movements such as writhing, twitching and shaking); when too little is transported, the patient re-enters the off state. Furthermore, as PD progresses, the therapeutic window for oral formulations of LD narrows, and it becomes increasingly difficult to control PD motor symptoms without inducing motor complications. In addition, most PD patients develop response fluctuations to intermittent oral LD therapy, such as end of dose wearing off, sudden on/offs, delayed time to on, and response failures.

Methods and devices that permit continuous or semi-continuous administration of drugs to subjects in order to produce reliable and steady pharmacokinetic performance are needed to improve efficacy and/or safety of therapies, particularly for drugs with a short half-life (e.g., in the plasma), and/or short persistence of the drug’s physiological effect, and/or a narrow therapeutic window.

### SUMMARY OF THE INVENTION

The present invention features a drug delivery device for continuously administering a pharmaceutical composition to the oral cavity, a storage case for said drug delivery device, and methods of using the drug delivery device to administer a drug to a patient.

In a first aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including: (a) a drug reservoir containing said pharmaceutical composition, the volume of said drug reservoir being from 0.1 mL to 5 mL; and (b) a reversibly compressible, multi-layered delivery tube in fluidic communication with the drug reservoir for delivery of the pharmaceutical composition to the mouth of the patient that is configured to recover its shape after compression such that the steady-state rate of administration of said pharmaceutical composition from said drug delivery device increases or decreases by less than 10% (e.g., less than 10%, less than 9%, less than 8%, less than 7%, less than 6%, less than 5%, less than 4%, less than 3%, less than 2%, or less than 1%) after said compression is released.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; (ii) a pump including: (a) a drug reservoir containing said pharmaceutical composition, the volume of said drug reservoir being from 0.1 mL to 5 mL; and (b) a delivery tube in fluidic communication with the drug reservoir for delivery of the pharmaceutical composition to the mouth of the patient; and (iii) a removable plug including an elastomeric polymer for hermetically sealing the delivery tube prior to initial use by the patient, wherein, following removal of the removable plug, the device is ready to be inserted into the mouth of the patient for said intraoral administration. In some embodiments, the delivery tube is a reversibly compressible, multi-layered delivery tube that is configured to recover its shape after compression such that the steady-state rate of administration of said pharmaceutical composition from said drug delivery device increases or decreases by less than 10% (e.g., less than 10%, less than 9%, less than 8%, less than 7%, less than 6%, less than 5%, less than 4%, less than 3%, less than 2%, or less than 1%) after said compression is released

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including a drug reservoir containing said pharmaceutical composition, the volume of said drug reservoir being from 0.1 mL to 5 mL; and in which the fastener includes a pocket that is fabricated as an integral part of the fastener for securing the drug delivery device to a buccal side of an upper molar of the patient, and wherein the pocket is configured to position the drug delivery device coplanar with an occlusal plane of the molar.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including a drug reservoir containing said pharmaceutical composition, the volume of said drug reservoir being from 0.1 mL to 5 mL, in which the drug reservoir comprises a rigid housing having a port for filling the drug reservoir with said pharmaceutical composition that is hermetically sealed with an elastomeric plug including a fluorocarbon elastomer that is biocompatible with the oral mucosa and compatible with the pharmaceutical composition.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including a drug reservoir containing said pharmaceutical composition, the volume of said drug reservoir being from 0.1 mL to 5 mL; in which the drug delivery device comprises two flow restrictors fluidically connected to the drug reservoir, and in which a first flow restrictor has a length of 6 - 12 mm and an internal diameter of 0.0100 - 0.0120 inches, and a second flow restrictor has a length of 14 — 24 mm and an internal diameter of 0.0130 — 0.0145 inches. In some embodiments, the two flow restrictors have the dimensions of a configuration listed in Table 3.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including: (a) a drug reservoir containing said pharmaceutical composition, the volume of said drug reservoir being from 0.1 mL to 5 mL; (b) a flow restrictor in fluidic communication with the drug reservoir; and (c) a reversibly compressible delivery tube in fluidic communication with the drug reservoir for delivery of the pharmaceutical composition to the mouth of the patient, in which said flow restrictor and/or said delivery tube are hermetically bonded either directly or indirectly to the drug reservoir by an adhesive.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a propellant-driven pump including a drug reservoir containing said pharmaceutical composition, the volume of said drug reservoir being from 0.1 mL to 5 mL; in which the propellant-driven pump comprises a rigid housing, an eyelet (e.g., an eyelet bonded to the housing), and a compressible, elastomeric septum for propellant loading fitted into the eyelet to form a hermetic seal.

In another aspect, the invention provides a drug delivery device configured to be removably inserted in a patient’s mouth and for continuous or semi-continuous intraoral administration of a pharmaceutical composition including a drug, said device including: (i) a first chamber containing the pharmaceutical composition; (ii) a second chamber containing a propellant; and (iii) a flexible and/or deformable diaphragm separating said first chamber from said second chamber, in which said first chamber, said second chamber, and, optionally, said diaphragm are bonded by an adhesive to form a hermetic seal.

In another aspect, the invention provides a drug delivery device configured to be removably inserted in a patient’s mouth and for continuous or semi-continuous intraoral administration of a pharmaceutical composition including a drug, said device including: (i) a first chamber containing the pharmaceutical composition; (ii) a second chamber containing a propellant; and (iii) a flexible and/or deformable diaphragm separating said first chamber from said second chamber, in which said first chamber, said second chamber, and, optionally, said diaphragm are partly or fully joined or sealed by a crimp.

In another aspect, the invention provides a drug delivery device configured to be removably inserted in a patient’s mouth and for continuous or semi-continuous intraoral administration of a pharmaceutical composition including a drug, said device including: (i) a first chamber containing the pharmaceutical composition; (ii) a second chamber containing a propellant; and (iii) a flexible and/or deformable diaphragm separating said first chamber from said second chamber, in which said first chamber comprises a first rigid housing, said second chamber comprises a second rigid housing, and in which the first chamber and the second chamber each include a rim having a step to strengthen the bond between, or the join formed upon the bonding of, the rims of the two chambers (e.g., by increasing the adhesive-bonded area).

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including a drug reservoir containing said pharmaceutical composition, the volume of said drug reservoir being from 0.1 mL to 5 mL; in which the fastener is configured such that the patient’s oral anatomy would mechanically interfere with and prevent the removal of the drug delivery device from the fastener when the fastener is attached to the surface of the patient’s mouth.

In another aspect, the invention provides a drug delivery device configured to be removably inserted in a patient’s mouth and for continuous or semi-continuous intraoral administration of a pharmaceutical composition including a drug, said device including: (i) a fastener to removably secure said drug delivery device to a surface of said patient’s mouth; and (ii) a pump including a drug reservoir containing said pharmaceutical composition, the volume of said drug reservoir being from 0.1 mL to 5 mL; in which the fastener is configured such that it has a friction fit with the drug delivery device and thereby prevents the drug delivery device from being dislodged from the fastener during insertion into the mouth or administration of the pharmaceutical composition. Alternatively, the drug delivery device is reversibly secured in the fastener with a detent, groove, snap, or lock.

In some embodiments of any of the foregoing aspects, the fastener secures the drug delivery device to a buccal side of an upper molar of the patient and is configured to position the drug delivery device coplanar with an occlusal plane of the molar. In some embodiments of any of the foregoing aspects, the fastener is configured to position the drug delivery device coplanar with the occlusal plane of the first or second molar. In some embodiments of any of the foregoing aspects, the fastener is configured to position the drug delivery device such that it only spans the first and second molars. In some embodiments of any of the foregoing aspects, the fastener is configured to position the drug delivery device such that a posterior end of the drug delivery device is between a midline and a posterior of the patient’s rear-most molar. In some embodiments of any of the foregoing aspects, the fastener is configured to position the drug delivery device such that it is set on the Wilson plane. In some embodiments of any of the foregoing aspects, the fastener includes a pocket that is fabricated as an integral part of the fastener for securing the drug delivery device to the buccal side of an upper molar of the patient.

In some embodiments of any of the foregoing aspects, the device further includes a reversibly compressible, multi-layered delivery tube in fluidic communication with the drug reservoir or first chamber for delivery of the pharmaceutical composition to the mouth of the patient that is configured to recover its shape after compression such that the steady-state rate of administration of said pharmaceutical composition from said drug delivery device increases or decreases by less than 10% (e.g., less than 10%, less than 9%, less than 8%, less than 7%, less than 6%, less than 5%, less than 4%, less than 3%, less than 2%, or less than 1%) after said compression is released.

In some embodiments of any of the foregoing aspects, administration of the pharmaceutical composition can be temporarily stopped when the drug delivery device is in use by compressing the delivery tube.

In some embodiments of any of the foregoing aspects, the delivery tube contains an outer layer and an inner layer containing laminated polymers, in which the outer layer contains an elastomeric polymer that is biocompatible with the oral mucosa. In some embodiments, the outer layer contains polyurethane, poly(ethylene glycol) diacrylate, or poly(lactide-co-glycolide) (PLGA). In some embodiments, the outer layer contains polyurethane. In some embodiments, the thickness of the outer layer is between 0.05 mm and 0.2 mm. In some embodiments, the inner layer contains a polymer that is compatible with the pharmaceutical composition and that provides radial strength and reduces water permeation. In some embodiments, the inner layer contains polyethylene terephthalate (PET), high density polyethylene (HDPE), polyvinylidene chloride (PVDC), polytetrafluoroethylene (PTFE), or polymeric fluorinated ethylene propylene (FEP). In some embodiments, the inner layer contains PET. In some embodiments, the thickness of the inner layer is between 0.025 mm and 0.075 mm. In some embodiments, the delivery tube substantially re-acquires its internal diameter after compression. In some embodiments, the delivery tube reduces loss of water from the pharmaceutical composition when it is in the delivery tube, thereby reducing drying out of the pharmaceutical composition. In some embodiments, the delivery tube has a length of 0.5 cm to 5 cm. In some embodiments, the delivery tube has an internal diameter of 0.010 inches to 0.080 inches.

In some embodiments of any of the foregoing aspects, the drug delivery device further contains a removable plug including an elastomeric polymer for hermetically sealing the delivery tube prior to initial use by the patient, and wherein, following removal of the removable plug, the device is ready to be inserted into the mouth of the patient for said intraoral administration.

In some embodiments of any of the foregoing aspects, the removable plug prevents extrusion of the pharmaceutical composition from the drug delivery device. In some embodiments of any of the foregoing aspects, the elastomeric polymer is compatible with the pharmaceutical composition. In some embodiments of any of the foregoing aspects, the removable plug includes a fluoropolymer. In some embodiments of any of the foregoing aspects, the removable plug includes a perfluoroelastomer (e.g., FFKM), a fluoroelastomer (e.g., FKM or FEPM), polybutadiene (BR), polychloroprene (CR), styrenebutadiene copolymer (SBR), nitrile rubber (NBR), hydrogenated nitrile rubber (HNBR), ethylene propylene rubber (EPM), ethylene propylene diene rubber (EPDM), chlorosulfonated polyethylene (CSM), or ethylene vinyl acetate rubber (EVA). In some embodiments of any of the foregoing aspects, the removable plug includes Viton™. In some embodiments of any of the foregoing aspects, the removable plug is encased in a heat shrink tubing to facilitate its removal from the drug delivery device. In some embodiments, the heat shrink tubing includes a fluoropolymer. In some embodiments of any of the foregoing aspects, the removeable plug and all or a portion of the delivery tube are enwrapped in a polymeric sheath prior to initial use of the drug delivery device by the patient. In some embodiments, the removeable plug and the entire length of the delivery tube are enwrapped in a polymeric sheath prior to initial use of the drug delivery device by the patient. In some embodiments, the heat shrink tubing is enwrapped in a polymeric sheath prior to initial use of the drug delivery device by the patient (e.g., the polymeric sheath enwraps the removable plug/heat shrink tubing assembly). In some embodiments, the polymeric sheath includes a fluoropolymer. In some embodiments, the polymeric sheath slows water vapor permeation, thereby reducing or preventing drying of pharmaceutical composition in the delivery tube (e.g., compared to drying in a delivery tube that is not enwrapped by the polymeric sheath). In some embodiments, the polymeric sheath slows oxygen permeation through the delivery tube. In some embodiments, the delivery tube is water impermeable when enwrapped by the polymeric sheath such that the pharmaceutical composition does not dry in the delivery tube. In some embodiments, the polymeric sheath includes a slit tail. In some embodiments, the tail of the polymeric sheath extends beyond the removable plug (or the removable plug/heat shrink tubing assembly) and applies retention force to maintain the removable plug inside the delivery tube. In some embodiments, the polymeric sheath is configured to be removed before insertion of the drug delivery device into the mouth of the patient. In some embodiments, the removable plug is configured to be removed before insertion of the drug delivery device into the mouth of the patient (e.g., by removal of the heat shrink tubing).

In some embodiments of any of the foregoing aspects, the drug delivery device further includes a fastener to removably secure said drug delivery device to a surface of said patient’s mouth. In some embodiments of any of the foregoing aspects, the fastener is configured such that the patient’s oral anatomy would mechanically interfere with and prevent the removal of the drug delivery device from the fastener when the fastener is attached to the surface of the patient’s mouth.

In some embodiments of any of the foregoing aspects, the fastener contains a pocket that is fabricated as an integral part of the fastener for securing the drug delivery device to a buccal side of an upper molar of the patient, and in which the pocket is configured to position the drug delivery device coplanar with an occlusal plane of the molar.

In some embodiments of any of the foregoing aspects, the pocket is configured to position the drug delivery device coplanar with the occlusal plane of the first or second molar. In some embodiments of any of the foregoing aspects, the pocket is configured to position the drug delivery device such that it only spans the first and second molars. In some embodiments of any of the foregoing aspects, the pocket is configured to position the drug delivery device such that a posterior end of the drug delivery device is between a midline and a posterior of the patient’s rear-most molar. In some embodiments of any of the foregoing aspects, the pocket is configured to position the drug delivery device such that it is set on the Wilson plane.

In some embodiments of any of the foregoing aspects, the fastener is worn over the bicuspids and molars. In some embodiments, the fastener is in a position spanning approximately halfway the hard and soft palates e.g., approximately half of the hard palate and half of the soft palate is covered by the fastener). In some embodiments of any of the foregoing aspects, the fastener is made of a plastic. In some embodiments, the plastic is an acrylate-containing polymer or an olefin-containing polymer. For example, the fastener may be made of a thermoplastic polyolefin, such as polypropylene. The plastic may be an Essix® PLUS™ plastic, an Essix A+® plastic, an Essix C+® plastic, an Essix ACE® plastic, an Essix® dual laminate plastic, an Essix® nightguard laminate plastic, an Essix® sports mouthguard material, an Essix® laminated sports mouthguard material, an Essix® bleach tray and model duplication material, an Essix Tray Rite® plastic, a Dreve Drufosoft® Pro plastic, a Dreve Kombiplast plastic, a Dreve Biolon plastic, or a Dreve Drufosoft® sports mouthguard material.

In some embodiments of any of the foregoing aspects, the pocket is configured to position the drug delivery device such that the pharmaceutical composition is released from a delivery tube on a lingual side of the patient’s teeth. In some embodiments of any of the foregoing aspects, the pocket is configured to position the drug delivery device such that the delivery tube wraps around the rear-most tooth of the patient. In some embodiments of any of the foregoing aspects, the pocket is configured such that the drug delivery device can be pushed into the pocket before the fastener is inserted into the mouth of the patient. In some embodiments of any of the foregoing aspects, the pocket is configured such that the drug delivery device cannot be dislodged from the pocket while being placed onto the surface of the patient’s mouth or during use in the mouth. In some embodiments of any of the foregoing aspects, the pocket is configured such that it has a friction fit with the drug delivery device and thereby prevents the drug delivery device from being dislodged from the pocket during insertion into the mouth or administration of the pharmaceutical composition. Alternatively, the drug delivery device is reversibly secured in the pocket with a detent, groove, snap, or lock. In some embodiments of any of the foregoing aspects, the pocket is configured such that the patient’s oral anatomy would mechanically interfere with and prevent the removal of the drug delivery device from the pocket when the fastener is attached to the surface of the patient’s mouth. In some embodiments of any of the foregoing aspects, the drug delivery device can be removed from the pocket by pushing on the device (e.g., when the device is outside of the mouth of a patient).

In some embodiments of any of the foregoing aspects, the fastener is matched to the dentition of the patient. In some embodiments of any of the foregoing aspects, the fastener is a retainer.

In some embodiments of any of the foregoing aspects, the drug reservoir or the first chamber includes a rigid housing having a port for filling the drug reservoir or chamber with said pharmaceutical composition that is hermetically sealed with an elastomeric plug including a fluorocarbon elastomer that is biocompatible with the oral mucosa and compatible with the pharmaceutical composition. In some embodiments, the elastomeric plug is an injection molded elastomeric plug. In some embodiments, the elastomeric plug is compressible. In some embodiments, the port sealed with the elastomeric plug can sustain a pressure of about 12 bar when the housing wall has a thickness of about 0.015 inches.

In some embodiments of any of the foregoing aspects, the drug delivery device includes two flow restrictors fluidically connected to the drug reservoir or first chamber, in which a first flow restrictor has a length of 6 - 12 mm and an internal diameter of 0.0100 - 0.0120 inches, and a second flow restrictor has a length of 14 — 24 mm and an internal diameter of 0.0130 — 0.0145 inches. In some embodiments, the two flow restrictors have the dimensions of a configuration listed in Table 3. In some embodiments, the pharmaceutical composition flows through both flow restrictors at approximately the same rate. In some embodiments, the pharmaceutical composition flows through both flow restrictors at different rates. In some embodiments, the pharmaceutical composition entering the first flow restrictor comes from a first region of the drug delivery device or first chamber and the pharmaceutical composition entering the second flow restrictor comes from a second region of the drug delivery device or first chamber. In some embodiments, the two flow restrictors are made of a chemically inert polymer with low water permeability. In some embodiments, the two flow restrictors are made of PET.

In some embodiments of any of the foregoing aspects, the drug delivery device includes a flow restrictor in fluidic communication with the drug reservoir or first chamber, and said flow restrictor and/or said delivery tube are hermetically bonded either directly or indirectly to the drug reservoir by an adhesive. In some embodiments, the flow restrictor is bonded to a coupling adaptor, which is then hermetically bonded to the drug reservoir or first chamber (e.g., the flow restrictor is bonded indirectly to the drug reservoir or first chamber). In some embodiments, the delivery tube is hermetically bonded to the drug reservoir or first chamber (e.g., the delivery tube is bonded directly to the drug reservoir or first chamber).

In some embodiments of any of the foregoing aspects, the pump is a mechanical pump, e.g., a propellant-driven pump. In another embodiment, the pump is an osmotic pump. In some embodiments, the pump is a battery-driven pump.

In some embodiments of any of the foregoing aspects, the second chamber or the propellant-driven pump contains a rigid housing, an eyelet (e.g., an eyelet bonded to the housing), and a compressible, elastomeric septum for propellant loading fitted into the eyelet to form a hermetic seal. In some embodiments, the septum is biocompatible with the oral mucosa and compatible with the propellant. In some embodiments, the septum includes nitrile rubber. In some embodiments, the septum forms a hermetic seal after a propellant-injecting needle is withdrawn from the septum. In some embodiments, the diameter of the septum is reduced upon compression. In some embodiments, the septum extends beyond the eyelet. In some embodiments, the septum is flared to prevent its ejection from the device under the pressure in the propellant chamber. In some embodiments, the eyelet includes a cut out to guide the propellant-injecting needle. In some embodiments, the eyelet includes an internal rib to increase resistance to injection.

In some embodiments of any of the foregoing aspects, said pump is a propellant-driven pump and the device includes: (i) a first chamber containing the pharmaceutical composition; (ii) a second chamber containing a propellant; and (iii) a flexible and/or deformable diaphragm separating said first chamber from said second chamber.

In some embodiments of any of the foregoing aspects, said first chamber, said second chamber, and, optionally, said diaphragm are bonded by an adhesive to form a hermetic seal.

In some embodiments of any of the foregoing aspects, the first chamber has a first rigid housing and the second chamber has a second rigid housing, and the first chamber and the second chamber each comprise a rim having a step for increasing the adhesive-bonded area (e.g., by strengthening the bond between, or the joint formed upon the bonding of, the rims of the two chambers). In some embodiments, the absolute propellant pressure is between 2 bars and 12 bars when the device is in the mouth at body temperature.

In some embodiments of any of the foregoing aspects, the adhesive is acrylate, gum, epoxy, or a sealant. In some embodiments, the adhesive is epoxy. In some embodiments, the epoxy adhesive includes two components, a first component containing a compound with two or more epoxide functions, and a second component containing a compound with two or more amine functions. In some embodiments, the cured adhesive is compatible with both the propellant and with components of the drug-containing fluid. In some embodiments, the first housing and the second housing sandwich the diaphragm.

In some embodiments of any of the foregoing aspects, said first chamber, said second chamber, and, optionally, said diaphragm are partly or fully joined or sealed by a crimp.

In some embodiments of any of the foregoing aspects, the first chamber has a first rigid housing and the second chamber has a second rigid housing, in which the first chamber and the second chamber each comprise a rim having a step, and in which the step of the rim of either the first housing or the second housing (e.g., the rim of the second housing) comprises crimped tabs.

In some embodiments of any of the foregoing aspects, the second chamber contains a propellant having a vapor pressure of about 4 atm or greater at 37° C.

In some embodiments of any of the foregoing aspects, the rigid housings are metallic. In some embodiments of any of the foregoing aspects, each of said rigid housings has a wall thickness between 0.25 mm and 0.5 mm.

In some embodiments of any of the foregoing aspects, the diaphragm is metallic. In some embodiments of any of the foregoing aspects, the diaphragm is 0.025 mm to 0.25 mm thick.

In some embodiments of any of the foregoing aspects, the pharmaceutical composition includes the drug and one or more excipients, and less than about 3% of said drug and of each of said excipients is absorbed by or transported through said delivery tube when the drug delivery device is stored non-frozen for one year. In some embodiments, the excipients include water, oil, and surfactant.

In some embodiments of any of the foregoing aspects, the device is positioned on the buccal side of the teeth of said patient. In some embodiments of any of the foregoing aspects, the delivery tube is configured to administer the pharmaceutical composition on the lingual side of the teeth of the patient.

In some embodiments of any of the foregoing aspects, the pump maintains an internal pressure of greater than or equal to about 2 bar on the pharmaceutical composition.

In some embodiments of any of the foregoing aspects, the pharmaceutical composition has a dynamic viscosity at 37° C. greater than 100 cP, such as greater than 10,000 cP, 50,000 cP, 100,000 cP, 500,000 cP, or 1,000,000 cP.

In some embodiments of any of the foregoing aspects, the pharmaceutical composition contains solid drug particles and/or solid excipient particles.

In another aspect, the invention provides a method of manufacturing a drug delivery device of the invention by applying an adhesive to any of said first chamber, said second chamber, said diaphragm, said flow restrictor, and/or said delivery tube, and curing said adhesive to form a hermetic seal.

In another aspect, the invention provides a storage case for temporarily stopping extrusion of a pharmaceutical composition from a drug delivery device, including paired components on opposing interior surfaces of the storage case that are configured to pinch or kink a reversibly compressible tube of a drug delivery device when the storage case is closed, thereby stopping extrusion from the drug delivery device. In some embodiments, the paired components comprise an elastomeric pad and a metal pin. In some embodiments, the storage case further includes a holder to align the drug delivery device in the storage case. In some embodiments, the storage case stops extrusion of a pharmaceutical composition from a drug delivery device of the invention. In some embodiments, wherein the case includes materials that do not support microbial growth. In some embodiments, the storage case is configured to temporarily stop extrusion from the drug delivery device while the drug delivery device is attached to a fastener configured to removably secure said drug delivery device to a surface of a patient’s mouth. In an alternative embodiment, the storage case is configured to temporarily stop extrusion from the drug delivery device while the drug delivery device is not attached to a fastener configured to removably secure said drug delivery device to a surface of a patient’s mouth.

In another aspect, the invention provides a method of pausing extrusion of a pharmaceutical composition from a drug delivery device of the invention by inserting the drug delivery device into a storage case of the invention and closing the lid of the storage case.

In another aspect, the invention provides a method of administering a pharmaceutical composition into a mouth of a patient by (i) inserting a drug delivery device of the invention into the mouth of said patient; (ii) administering said pharmaceutical composition to said patient; and (iii) removing said device from the mouth of said patient. In some embodiments, the method further includes temporarily stopping administration of the pharmaceutical composition by pinching or kinking the delivery tube. In some embodiments, the tube is pinched or kinked by inserting the drug delivery device into a storage case of the invention and closing the lid.

In some embodiments of any of the foregoing methods, the method further includes first inserting the pump for delivery of said pharmaceutical composition into the fastener (e.g., into the pocket of the fastener) configured to removably secure said drug delivery device to the surface of the patient’s mouth (e.g., before insertion in the mouth or before insertion into the storage case).

In another aspect, the invention provides a packaging material for long term storage of a drug delivery device including a hermetically sealed pouch that is gas impermeable with an atmosphere that is substantially free of oxygen. In some embodiments, the pouch is filled with an inert gas. In some embodiments, the inert gas is nitrogen, argon, or carbon dioxide. In some embodiments, a wall of the pouch includes a laminate of a metal film and a polymer. In some embodiments, the pouch comprises a humidifying porous insert. In some embodiments, the pouch comprises a drug device described herein.

In some embodiments, the drug delivery device configured to be removably inserted in a patient’s mouth and for continuous or semi-continuous intraoral administration of a drug includes a propellant-driven pump including a rigid housing, the rigid housing including a wall of a first chamber containing a drug-including fluid and a wall of a second chamber containing a propellant. The device can include a flexible and/or deformable propellant-impermeable diaphragm separating the first chamber from the second chamber. The diaphragm can include a wall of the first chamber and a wall of the second chamber. In particular embodiments, the density of a metal comprising or constituting the propellant-impermeable diaphragm can be greater than 2.0 g per cm³ at 25° C. The diaphragm can be metallic (e.g., tin or silver or aluminum or iron or magnesium or copper or titanium or steel or an alloy of tin or of silver or of aluminum or of iron or of magnesium or of copper or of titanium). Optionally, the metallic diaphragm can comprise silver or an alloy of silver, or iron or an alloy of iron. The diaphragm can be shaped to substantially conform to the interior housing wall of the first chamber and/or the interior housing wall of the second chamber. The diaphragm can be between 10 µm and 250 µm thick, e.g., between 20 µm and 125 µm thick, such as between 25 µm and 75 µm thick. In particular embodiments, the thickness of the diaphragm can vary across the interior of the housing by less than ± 25 %, or by less than ± 10 %. In other embodiments, the diaphragm includes a rim that is thicker than the center of the diaphragm (e.g., the thickness of the rim can be at least 1.5 times greater than the thickness of the center of the diaphragm, the thickness of the rim can be between 1.5 times and 2 times the thickness of the center of the diaphragm, the thickness of the rim can be between 2 times and 3 times the thickness of the center of the diaphragm, or the thickness of the rim can be 3 times or more the thickness of the center of the diaphragm). The diaphragm can be planar, folded, pleated, or scored. The device can be hermetically sealed except for one or more orifices for drug filling or drug delivery. Optionally, the one or more orifices for drug filling or drug delivery can be hermetically or non-hermetically sealed. Optionally, the one or more orifices for drug filling or delivery are hermetically sealed. In particular embodiments, the propellant chamber can be hermetically sealed and can include a hermetically sealed orifice for filling with propellant. In certain embodiments, the drug chamber can include two, three, or more hermetically sealable or sealed orifices for filling with drug or for drug delivery. In still other embodiments, the rigid housing and the diaphragm can be joined by a hermetically sealing weld. For example, the hermetically sealing weld can prevent the influx of air and water vapor or the outflux of water vapor, drug or propellant, or prevent the influx of air or oxygen, or prevent the influx or the outflux of helium. In particular embodiments, the rigid housing of the device can include a metal, a ceramic, or a composite of a polymer reinforced by fibers (e.g., carbon fibers, glass fibers, or metal fibers). The rigid housing can include a material having at 25±3° C. a yield strength greater than 100 MPa, and/or having at 25±3° C. a tensile yield strength greater than 100 MPa, and/or having at 25±3° C. a modulus of elasticity greater than 30 GPa, and/or having at 25±3° C. a Brinell hardness greater than 200 MPa, and/or having a density greater than 2.5 g/cm³ at 25±3° C., e.g., greater than 3.5 g/cm³ such as greater than 4.5 g/cm³, or having a density equal to or greater than 5.5 g/cm³. The rigid housing can include a metal selected from the group titanium or iron or aluminum or molybdenum or tungsten or an alloy of titanium or iron or aluminum or molybdenum or tungsten. In some embodiments, the rigid housing is formed of steel, such as stainless steel. In particular embodiments, the rigid housing can include titanium or an alloy of titanium and a metallic diaphragm (that can separate chambers within the housing) can be welded to the rigid housing including titanium or an alloy of titanium. In certain embodiments, the diaphragm can include silver or an alloy of silver or it can optionally include iron or an alloy of iron. In some embodiments, the diaphragm can include iron or tin or silver or titanium, or an alloy of iron or of tin or of silver or of titanium. In some embodiments, the diaphragm can include steel, such as stainless steel.

In some embodiments, the drug delivery device configured to be removably inserted in a patient’s mouth and for continuous or semi-continuous intraoral administration of a drug includes: a first chamber containing a drug-including fluid; a second chamber containing a propellant; and a flexible and/or deformable diaphragm separating the first chamber from the second chamber, wherein 75% - 85%, 86% -95%, or >95% of the drug-including fluid can be dispensed while the delivery rate can vary by less than ±20%, ±15%, ±10%, or ±5%, over a period of at least 4, 8, 16, or 24 hours. The pump can include a liquid propellant, the liquid propellant having a boiling point of less than 37° C. at sea level atmospheric pressure. In particular embodiments, the liquid propellant can be a hydrocarbon, a halocarbon, a hydrofluoroalkane, an ester, or an ether (e.g., the liquid propellant can be 2-methylpropane, isopentane, trifluorochloromethane, dichlorofluoromethane, 1-fluorobutane, 2-fluorobutane, 1,2-difluoroethane, dimethyl ether, 2-butene, n-butane, 1-fluoropropane, 1-butene, 2-fluoropropane, 1,1-difluoroethane, cyclopropene, propane, propene, or diethyl ether). In certain embodiments, the liquid propellant is 1,1,1,2-tetrafluoroethane, 1,1,1,2,3,3,3-heptafluoropropane, 1,1,1,3,3,3-hexafluoropropane, octafluorocyclobutane or isopentane. The propellant can have a vapor pressure of greater than 1.5 bar and less than 20 bar at 37° C., such as a vapor pressure of greater than 2.0 bar and less than 15 bar at 37° C., or a vapor pressure of greater than 3.0 bar and less than 10 bar at 37° C. The drug delivery device can include a reservoir containing any pharmaceutical composition described herein.

In some embodiments, the drug delivery device configured to be removably inserted in a patient’s mouth and for continuous or semi-continuous intraoral administration of a drug includes: (i) a fastener to removably secure the drug delivery device to a surface of the patient’s mouth; (ii) a mechanical pump; and (iii) a drug reservoir containing any of the pharmaceutical compositions of the invention, the volume of the drug reservoir being from 0.1 mL to 5 mL (e.g., from 0.1 mL to 4 mL, from 0.1 mL to 3 mL, from 0.1 mL to 2 mL, from 0.1 mL to 1 mL, from 0.1 mL to 0.5 mL, from 0.1 mL to 0.25 mL, from 0.2 mL to 5 mL, from 0.3 mL to 5 mL, from 0.5 mL to 5 mL, from 1 mL to 5 mL, from 2 mL to 5 mL, from 4 mL to 5 mL, from 0.5 mL to 1 mL, from 0.5 mL to 2 mL, from 1 mL to 2 mL, from 2 mL to 3 mL).

A drug delivery device of the invention may removably secure to one or more teeth of the patient. In some embodiments, the fastener that removably secures the drug delivery device to one or more teeth includes a band, a bracket, a clasp, a splint, or a retainer. For example, the fastener may include a transparent retainer or a partial retainer attachable to fewer than 5 teeth.

In some embodiments, a drug delivery device of the invention further includes one, two, or more flow restrictors (e.g., a tube or orifice). The flow restrictor can have an internal diameter smaller than 1 mm or 2 mm and larger than 0.05 mm and a length between 0.25 cm and 10 cm. In particular embodiments, the flow restrictor can have an internal diameter smaller than 0.7 mm and larger than 0.2 mm. Preferred internal diameters are 0.1 — 2 mm (0.1 - 0.7 mm, 0.2 - 0.5 mm, 0.5 - 0.75 mm, 0.75 - 1.0 mm, 1.0 - 1.5 mm, or 1.5 - 2.0 mm) and preferred lengths are 0.25 - 5 cm (such as 1 - 2.5 cm, 1 - 5 cm, 0.25 - 0.5 cm, 0.5 - 0.75 cm, 0.75 - 1 cm, 1 - 2 cm, 2 - 3 cm, 3 - 4 cm, or 4 - 5 cm). The flow restrictor can be made of a plastic, such as an engineering plastic. In particular embodiments, the engineering plastic includes a polyamide or a polyester, or a polycarbonate, or a polyetheretherketone, or a polyetherketone, or a polyimide, or a polyoxymethylene, or a polyphenylene sulfide, or a polyphenylene oxide, or a polysulfone, or polytetrafluoroethylene, or polyvinylidene difluoride, or ultra-high-molecular-weight polyethylene, or a strong elastomer.

Any of the drug delivery devices of the invention may be configured to deliver an average hourly rate of volume of from about 0.015 mL/hour to about 1.25 mL/hour over a period of from about 4 hours to about 168 hours at about 37° C. and a constant pressure of about 1.013 bar, wherein the average hourly rate varies by less than ± 20% or ± 10% per hour over a period of 4 or more hours (e.g., 6 hours, 8 hours, 10 hours, 12 hours, 18 hours, 24 hours, 36 hours, 48 hours, 60 hours, 72 hours, 168 hours, or more).

Any of the drug delivery devices of the invention may be configured to continuously or semicontinuously administer the pharmaceutical composition into the mouth of the patient at a rate between 0.001 mL/hour and 1.25 mL/hour

In another aspect, the invention features a method of administering a pharmaceutical composition to a patient, the method including removably attaching a drug delivery device of the invention to an intraoral surface of the patient. In certain embodiments, the method further includes detaching the device from the intraoral surface and/or administering drug to the patient for a delivery period of not less than about 4 hours and not more than about 7 days. In some embodiments, the drug delivery device includes a drug reservoir including a volume of a drug, and the method further includes oral administration at a rate in the range of from 15 µL per hour to about 1.25 mL per hour (e.g., as described herein) during the delivery period. For example, the method can include oral administration at a rate in the range of from about 0.015 mL/hour to about 0.25 mL/hour; from about 0.25 mL/hour to about 0.5 mL/hour; from about 0.5 mL/hour to about 0.75 mL/hour; or from about 0.75 mL/hour to about 1.0 mL/hour. In particular embodiments, the fluctuation index of the drug is reduced to less than or equal to 2.0, 1.5, 1.0, 0.75, 0.50, 0.25, or 0.15 during the delivery period. In certain embodiments, the pharmaceutical composition can be administered to the patient over a delivery period of about 4 or more hours (e.g., 4, 8, 10, 12, 14, 16, 18, 20, 24, or more hours).

In certain embodiments, a method of administering a pharmaceutical composition to a patient further includes treating a disease in the patient, wherein the disease is spasticity, muscle weakness, mucositis, allergy, an immune disease, anesthesia, a bacterial infection, cancer, pain, organ transplantation, disordered sleep, epilepsy or a seizure, anxiety, a mood disorder, post-traumatic stress disorder, arrhythmia, hypertension, heart failure, or diabetic nephropathy.

In one particular embodiment of any of the above methods, the method further includes treating a disease in the patient, wherein the disease is multiple sclerosis, cerebral palsy, spasticity, neurogenic orthostatic hypotension, Wilson’s disease, cystinuria, rheumatoid arthritis, Alzheimer’s disease, myasthenia gravis, Type-1 Gaucher disease, Type C Niemann-Pick disease, eosinophilic gastroenteritis, chronic mastocytosis, ulcerative colitis, gastro-oesophageal reflux, gastroenteritis, hyperemesis gravidarum, glioblastoma multiforme, anaplastic astrocytoma, pulmonary hypertension, coronary heart disease, congestive heart failure, angina, Type 2 diabetes, COPD, asthma, irritable bowel syndrome, overactive bladder, or urinary urge incontinence. In one particular embodiment, the method includes treating myasthenia gravis and the pharmaceutical composition includes pyridostigmine, or a pharmaceutically acceptable salt thereof.

In one particular embodiment of treating a disease in a patient, the method includes treating Parkinson’s disease and the pharmaceutical composition includes levodopa or a levodopa prodrug. In some embodiments, the pharmaceutical composition further comprises carbidopa or a carbidopa prodrug and/or benserazide.

In any of the devices or methods of the invention, the pharmaceutical composition may include one or more of levodopa or a levodopa prodrug, baclofen or a baclofen prodrug, pyridostigmine or a pyridostigmine prodrug, pilocarpine or a pilocarpine prodrug, furosemide or a furosemide prodrug, methylphenidate, a prostaglandin, prostacyclin, treprostinil, beraprost, nimodipine, and testosterone.

In any of the preceding embodiments of the above devices and methods, the pharmaceutical composition may include a mucoadhesive polymer and, optionally, a permeation enhancer (e.g., to aid transport across the sublingual or buccal mucosa).

In particular embodiments of treating a disease in a patient: (a) the patient has Parkinson’s disease, (b) the drug reservoir includes levodopa or a levodopa prodrug, and (c) the method includes administering into the patient’s mouth the levodopa or a levodopa prodrug for a period of at least 4 hours at an hourly rate in the range of 30 mg/hour to 150 mg/hour. The method can include intraoral administration such that a circulating plasma levodopa concentration greater than 1,200 ng/mL and less than 2,500 ng/mL is continuously maintained for a period of at least 4 hours during the administration. In certain embodiments, the subject has a score of 4 and 5 on the Hoehn and Yahr scale.

In a method for treating Parkinson’s disease in a patient, the fluctuation index of levodopa may be less than or equal to 2.0, 1.5, 1.0, 0.75, 0.50, 0.25, or 0.15 for a period of at least 4 hours (e.g., at least 6 hours, at least 8 hours, or longer) during the administration. In some embodiments, during administration the circulating levodopa plasma concentration varies by less than +/- 20% or +/- 10% from its mean for a period of at least 1 hour (e.g., 2 hours, 3 hours, 4 hours, or more hours).

In a further aspect, the invention features a method for treating Parkinson’s disease in a patient, the method including continuous or semi-continuous administration of a pharmaceutical composition comprising levodopa or a levodopa prodrug using a drug delivery device described herein into the patient at a rate of 10 mg/hour to 200 mg/hour (e.g., as described herein, such as 30 mg/hour to 150 mg/hour or 50 mg/hour to 125 mg/hour) for a period of about 4 hours to about 168 hours (e.g., as described herein).

In some embodiments of methods of treating Parkinson’s disease, the patient has a motor or non-motor complication of Parkinson’s disease such as a complication including tremor, akinesia, bradykinesia, dyskinesia, dystonia, cognitive impairment, or disordered sleep. In particular embodiments, the method of treating Parkinson’s disease includes treating a motor or non-motor complication of Parkinson’s disease.

In certain embodiments of treating a disease, the method includes treating spasticity in a subject in need thereof and the pharmaceutical composition includes baclofen or a baclofen prodrug. In some embodiments, the subject has multiple sclerosis or a spinal cord injury.

In certain embodiments of treating a disease, the method includes treating myasthenia gravis in a subject in need thereof, and the pharmaceutical composition includes pyridostigmine or a pyridostigmine prodrug.

In certain embodiments of treating a disease, the method includes treating dry mouth in a subject in need thereof and the pharmaceutical composition includes pilocarpine or a pilocarpine prodrug. In some embodiments, the dry mouth is associated with or caused by Sjogren’s syndrome or radiation therapy.

In certain embodiments of treating a disease, the method includes treating high blood pressure (hypertension) or edema in a subject in need thereof, and the pharmaceutical composition includes furosemide or a furosemide prodrug. In some embodiments, the edema is associated with or caused by heart failure (e.g., congestive heart failure), kidney disease, or liver disease (e.g., cirrhosis or liver fibrosis).

In one embodiment of any of the above methods, during the administration the circulating drug plasma concentration varies by less than +/- 20% or +/- 10% from its mean for a period of at least 1, 2, or 4 hours.

In still other embodiments, the drug reservoir includes a composition including a suspension that is a drug particle-containing emulsion including (i) from 35% to 70% (w/w) drug particles including levodopa and/or carbidopa, or salts thereof, (ii) from 19% to 30% (w/w) of one or more water-immiscible compounds (e.g., an oil), (iii) from 2% to 16% (w/w) water, and (iv) from 1% to 8% (w/w) surfactant. The suspension can include a continuous hydrophilic phase including greater than 50% (w/w) drug particles.

In an embodiment of any of the above devices, methods, and pharmaceutical compositions, the drug can be an analgesic (e.g., lidocaine, bupivacaine, mepivacaine, ropivacaine, tetracaine, etidocaine, chloroprocaine, prilocaine, procaine, benzocaine, dibucaine, dyclonine hydrochloride, pramoxine hydrochloride, benzocaine, proparacaine, and their pharmaceutically acceptable salts) or an opioid (e.g., buprenorphine, nor-buprenorphine, fentanyl, methadone, levorphanol, morphine, hydromorphone, oxymorphone codeine, oxycodone, hydrocodone, and their pharmaceutically acceptable salts) administered for the treatment of pain.

In some embodiments of any of the above aspects, the drug delivery device configured for continuously or semi-continuously administering a drug into the mouth of a patient includes: a pharmaceutical composition including a paste, solution or suspension having a viscosity greater than 100 poise and less than 100,000 poise at 37° C. and including the drug; and a mechanical pump including a flow restrictor, the flow restrictor including an internal diameter between 0.05 mm and 3.00 mm and a length between 0.25 cm and 20 cm, configured and arranged to administer the pharmaceutical composition at a rate between 0.001 mL/hour and 1.25 mL/hour. The mechanical pump can include a propellant. In particular embodiments, the propellant has a vapor pressure at about 37° C. greater than 2 bar and less than 50 bar. The pharmaceutical composition includes solid drug particles and/or excipient particles can have a D₉₀ between 0.1 µm and 200 µm and a D₅₀ between 0.1 µm and 50 µm when measured by light scattering with the particles dispersed in a non-solvent. The drug delivery device of can be configured such that: (i) the administration rate is greater than 0.03 mL/hour and less than 0.5 mL/hour; (ii) the viscosity greater than 200 poise and less than 100,000 poise; (iii) the flow restrictor has an internal diameter between 0.1 mm and 0.7 mm and a length between 1 cm and 5 cm; and (iv) the propellant has a vapor pressure at about 37° C. greater than 2.5 bar and less than 15 bar. In particular embodiments, the solid drug particles and/or excipient particles having a D₉₀ between 1 µm and 50 µm and a D₅₀ between 0.5 µm and 30 µm when measured by light scattering with the particles dispersed in a non-solvent. The drug delivery device of can be configured such that: (i) the administration rate is greater than 0.05 mL/hour and less than 0.2 mL/hour; (ii) the viscosity is greater than 500 poise and less than 100,000 poise; (iii) the flow restrictor has an internal diameter between 0.2 mm and 0.5 mm and a length between 1 cm and 2.5 cm; and (iv) the propellant has a vapor pressure at about 37° C. greater than 4 bar and less than 10 bar. In particular embodiments, the solid drug particles and/or excipient particles having a D₉₀ between 3 µm and 30 µm and a D₅₀ between 2 µm and 20 µm when measured by light scattering with the particles dispersed in a non-solvent.

### DEFINITIONS

The term “about,” as used herein, refers to a number that is ±10% of a value that this term precedes except when the value is that of a temperature. For temperatures “about” means ± 3° C.

The term “administration” or “administering” refers to a method of giving a dosage of a therapeutic drug, such as LD and/or carbidopa (CD), to a patient. The drug may be formulated as a fluid, such as a viscous suspension. Fluids may be infused. The dosage form of the invention is preferably administered into the mouth or nasal cavity, optionally using a drug delivery device such as an infusion pump, and the drug can be swallowed and/or absorbed anywhere within the mouth or via the stomach. Typical durations of administration from a single device or drug reservoir are greater than 4, 8, 12, or 16 hours per day, up to and including 24 hours per day. Administration can also take place over multiple days from a single device or drug reservoir, e.g., administration of a drug for 2 or more days, 4 or more days, or 7 or more days.

The term “CD” refers to Carbidopa.

The term “compatible” means that a material contacting a pharmaceutical composition does not substantially change the chemical, physical, or pharmacological properties of the pharmaceutical composition.

As used herein, “co-administered” or “co-infused” refers to two or more pharmaceutically active agents, formulated together or separately, and administered or infused into the mouth simultaneously or within less than 60, 30, 15, or 5 minutes of each other.

The term “COMT” refers to catechol-O-methyl transferase.

As used herein “continuous administration” or “continuous infusion” refers to uninterrupted administration or infusion of a drug in solid or fluid form.

As used herein the term “Dso” is defined as the median for a volume distribution (as opposed to a mass, number, or surface distribution) of the particles. The particle size can be measured by conventional particle size measuring techniques well known to those skilled in the art. Such techniques include, for example, optical microscopy, electron microscopy, sedimentation, field flow fractionation, photon correlation spectroscopy, light scattering (e.g., with a Microtrac UPA 150, Malvern Mastersizer), laser diffraction, and centrifugation. D₅₀ values are commonly derived of particle size distributions of particles suspended in a non-solvent, the distributions measured by light scattering.

The term “DDC” refers to DOPA decarboxylase.

As used herein, the term “drug particle” refers to solid particles including a drug. The drug particles can be included in the pharmaceutical compositions of the invention. For example, the pharmaceutical composition can contain particulates containing or formed from LD, LD salts, CD, or CD salts.

As used herein the term “engineering plastic” is synonymous with the terms “engineered plastic”, “engineered polymer” and “engineering polymer”. The term means a polymer differing from the most widely used polymers in its superior mechanical properties, or in its superior resistance to chemicals or its lesser wetting by water or by oils, or its lesser swelling in water or in oils. Exemplary engineering plastics include polyamides such as Nylon 6, Nylon 6-6 and other Nylons; polyesters like polybutylene terephthalate or polyethylene terephthalate; polycarbonates; polyetheretherketones; polyetherketones; polyimides; polyoxymethylenes such as polyacetals or polyformaldehydes; polyphenylene sulfide; polyphenylene oxide; polysulfone; polytetrafluoroethylene; polyvinylidene difluoride; ultra-high-molecular-weight polyethylene; and strong elastomers such as highly crosslinked acrylonitrile butadiene styrene, and their co-polymers.

As used herein, the term “fastener” refers to an element for attaching the device of the invention, or its components, to a surface of the mouth (e.g., to the teeth). Exemplary methods of attachment are fasteners banded, adhered, cemented or glued to one, two or more teeth; dental appliances; splints; transparent retainers; metal wire Hawley retainers; partial retainers on one side of the mouth (e.g., attached to 3, 4, or 5 teeth); thermo or vacuum-formed Essix retainers typically including a polypropylene or polyvinylchloride material, typically 0.020” or 0.030” thick; thermo-formed Zendura retainers including polyurethane; bonded (fixed) retainers including a passive wire bonded to the tongue-side of lower or upper teeth; muco-adhesives that adhere to the oral mucosal tissue and slowly erode; and fasteners that conform or are molded to fit a patient’s teeth or soft tissue, similar to dental splints used to treat bruxism and sleep apnea. Similarly, the drug delivery devices, drug pumps, drug reservoirs and other devices of the invention may be directly or indirectly attached to a removable denture, a prosthetic tooth crown, a dental bridge, a moral band, a bracket, a mouth guard, or a dental implant.

As used herein the term “fluctuation index” refers to the magnitude of the rise and fall of drug level in plasma relative to the average plasma concentration, and is defined as [Cₘₐₓ-Cₘᵢₙ]/ C_{avg}. The fluctuation index is measured over a specified period of time. The time period can begin, for example, after the drug’s plasma concentration: has reached the steady-state concentration; or has reached 90% of the steady-state concentration; or 30, 60, or 120 minutes after any of the drug delivery devices of the invention has been inserted into the mouth and begun to deliver drug. The time period can end, for example: at the end of the use period specified in the instructions for use of the drug delivery device; when the drug reservoir is 90% depleted or substantially depleted; or about 4, 8, 16, 24, 72, or 168 hours after the start of the time period.

As used herein, the term “fluid” encompasses any drug-including liquid, gel, or non-pourable suspension that can be pumped or extruded. The fluid can be a Newtonian or a non-Newtonian fluid; it can be an easy to deform solid or a soft paste, which may move as a plug via slip flow. It can be, for example, a viscous Newtonian or non-Newtonian suspension. The term encompasses, for example, true solutions, colloidal solutions, emulsions, pastes, suspensions, and dense semi-solid toothpaste-like suspensions deforming under pressure sufficiently to be extruded into the mouth. The fluid infused can be aqueous, non-aqueous, single phase, two-phase, three- phase or multiphase. The emulsions can be, for example, oil-in-water or water-in-oil, and can include micelles and/or liposomes.

As used herein, “infused” or “infusion” includes infusion into any part of the body, preferably infusion into the mouth or nasal cavity. It is exemplified by extrusion into the mouth.

The term “LD” refers to levodopa, also known as L-DOPA, or a salt thereof.

The term “MAO-B” refers to monoamine oxidase-B.

As used herein, “mechanical pump” means any drug delivery device whose motive force is not electricity, magnetism, or gravity. Examples of mechanical pumps include drug delivery devices wherein the drug is delivered by the force or pressure of a spring, an elastomer, a compressed gas, or a propellant.

As used herein, “mouth” includes the areas of the oral cavity, including those areas of the oral cavity adjacent the lips, cheeks, gums, teeth, tongue, roof of the mouth, hard palate, soft palate, tonsils, uvula, and glands.

The abbreviation “M” means moles per liter. Usage of the term does not imply, as it often does in chemistry, that the drug is dissolved. As used herein 1 M means that a 1 liter volume contains 1 mole of the combination of the undissolved (often solid) and/or the dissolved drug. For example, 1 M LD means that there is 197 mg of solid (undissolved) and dissolved LD in one mL.

The term “PD” refers to Parkinson’s disease.

The term “prodrug,” as used herein, represents compounds that are rapidly transformed in vivo to the parent compound, for example, by hydrolysis in blood. Prodrugs may be conventional esters. Some common esters that have been utilized as prodrugs are phenyl esters, aliphatic (C₈-C₂₄) esters, acyloxymethyl esters, carbamates, and amino acid esters. For example, a compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26:4351-4367, 1996, each of which is incorporated herein by reference.

As used herein, “pump” refers to any mechanism capable of administering a fluid formulated drug product over a period of 4 or more hours. Examples of pumps include battery-powered pumps (e.g., syringe pumps, piezoelectric, peristaltic pumps, or diaphragm pumps), mechanical devices with or without moving parts that are not battery-powered (e.g., liquefied propellant driven pumps, gas-driven pumps, spring-driven pumps, shape memory alloy driven pumps, and elastomeric pumps), osmotic pumps, and battery-operated electroosmotic pumps (with or without moving parts).

The terms “semi-continuous administration” and “frequent administration,” as used interchangeably herein, refer to an administration (e.g., infusion) of a drug in solid or fluid form at a frequency of at least once every 120 minutes, and preferably at least every 90, 60, 30, 15, or 5 minutes.

As used herein, the term “shelf life” means the shelf life of the drug delivered by the inventive device (e.g., LD or CD), in its form as a product sold for use by consumers, during which period the product is suitable for use by a patient. The shelf life of the drugs (e.g., LD or CD) administered by the devices of the invention can be greater than 3, 6, 12, 18, or preferably 24 months. The shelf life may be achieved when the product is stored frozen (e.g., at about -18° C.), stored refrigerated (at 5 ± 3° C., for example at 4 ± 2° C.), or stored at room temperature (e.g., at about 25° C.). The drug (e.g., LD or CD) product sold to consumers may be the drug-containing suspension, e.g., suspension ready for infusion, or it may be its components.

As used herein, “stable” refers to stable formulations of any of the drugs administered by the devices of the invention. Stable formulations exhibit physical stability (as defined above) and a reduced susceptibility to chemical transformation (e.g., oxidation) prior to administration into a patient. Stable drug formulations have a shelf life at about 5° C. and/or at about 25° C. of equal to or greater than 3, 6, 12, 18, or 24 months, and an operational life of greater than or equal to 8 hours, 12 hours, 16 hours, 24 hours, 48 hours, 72 hours, 96 hours, or 7 days. In the context of LD and/or CD containing formulations, “stable” refers to formulations which are chemically stable and physically stable. Chemically stable formulations are those having a shelf life during which less than 20% (e.g., 10%, 5%, 4%, 3%, 2% or less than 1%) of the LD and/or CD is chemically transformed (e.g., oxidized) when stored for a period of 3, 6, 12, 18, or 24 months. For formulations such as suspensions and drug particle-containing emulsions, the term “stable” also refers to formulations that are physically stable. In the context of LD and CD, “stable” refers to formulations that are “oxidatively stable.” Stable formulations of LD and CD are those having a shelf life during which less than 10% (e.g., 5%, 4%, 3%, 2% or less than 1%) of the LD and CD is oxidized when stored for a period of 3, 6, 12, 18, or 24 months. Stable formulations of LD and CD have an operational life during which less than 10% (e.g., as described herein) of the LD and CD is oxidized over a period of 8 hours, 12 hours, 16 hours, 24 hours, 48 hours, 72 hours, 96 hours, or 7 days. The chemically stable formulations may contain less than 1 µg of hydrazine per mg of CD when stored for a period of 3, 6, 12, 18, or 24 months at about 5° C. and/or at about 25° C.

As used herein, “substantially free of oxygen” refers to compositions packaged in a container for storage or for use wherein the packaged compositions are largely free of oxygen gas (e.g., less than 10%, or less than 5%, of the gas that is in contact with the composition is oxygen gas) or wherein the partial pressure of the oxygen is less than 15 torr, 10 torr, or 5 torr. This can be accomplished, for example, by replacing a part or all of the ambient air in the container with an inert gas, such as nitrogen, carbon dioxide, argon, or neon.

As used herein, the term “suitable for continuous or frequent intermittent intra-oral delivery” refers to drug particle suspensions of the invention that are efficacious and safe upon intra-oral delivery. For example, local adverse events in or near the mouth (if any) produced by continuous or frequent intermittent intra-oral administration of the suspension are tolerable or mild.

As used herein the term “suspension” refers to a mixture including a liquid and particles of at least one solid. The liquid can be aqueous or non-aqueous or an emulsion. The non-aqueous liquid can be an edible oil and the emulsion can include an edible oil. Suspensions may be, for example, flowing suspensions or suspensions that are extruded, i.e., slipping as a plug (e.g., through a flow-controlling orifice, nozzle, or tubing).

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to ameliorate the disease and improve the patient’s condition. The term “treating” also includes treating a patient to delay progression of a disease or its symptoms. Thus, in the claims and embodiments, treating is the administration to a patient either for therapeutic or prophylactic purposes.

As used herein “viscosity” means dynamic viscosity also known as shear viscosity, measured at a low shear rate.

Other features and advantages of the invention will be apparent from the following Detailed Description, the drawings, and the claims.

### BRIEF DESCRIPTION OF THE DRAWINGS

FIG. 1 is a drawing showing an exploded view of components of a device for constant rate, continuous extrusion of a drug-containing fluid into the mouth. Components shown in FIG. 1 include propellant housing 1 and drug housing 3, both of which can be made of or contain metal, and diaphragm 2, which may be made of metal or contain a metal film. Septum eyelet 4 is inserted into propellant housing 1 and can be made of metal, typically titanium or titanium alloy. Septum 5 can be inserted into septum eyelet 4, and may optionally contain a rubber, such as nitrile rubber. Elastomeric plug 6 can be inserted into drug housing 3 to seal the drug chamber. Distal, long flow restrictor 7a is mounted in coupling adaptor 8, and delivers drug to delivery tube 9 and 10 (labeled with two numbers to indicate that it is a two-layered delivery tube). Delivery tube 9 and 10 can contain removable plug 12, which can seal the delivery tube and prevent extrusion of drug-containing fluid when the drug delivery device is stored before use. Removable plug 12 is encased in heat shrink tubing 13. Delivery tube 9 and 10 and removable plug 12 encased in heat shrink tubing 13 are enwrapped by polymeric sheath 11 to reduce oxygen and water permeation through the delivery tube and maintain the removable plug inside the delivery tube.

FIG. 2 is a drawing showing parts of an assembly for constant rate, continuous extrusion of a drug-containing fluid into the mouth. FIG. 2 shows an embodiment of the device of FIG. 1 that contains two flow restrictors 7 of different lengths, in which one is substantially shorter than the other (e.g., one flow restrictor extends further into the drug chamber than the other).

FIGS. 3A-3B are a series of diagrams showing views of the delivery tube assembly. FIG. 3A shows a cutaway view of the delivery tube assembly (top) and a view of an intact delivery tube assembly (bottom), each including a laminated delivery tube, removable plug, heat shrink tubing, and polymeric sheath. FIG. 3B shows an enlarged view of the cutaway view of the delivery tube of FIG. 3A (the area that is enlarged is indicated by a circle in the schematic in the upper right corner).

FIG. 4 is a drawing showing a cross-sectional view of a multilayered delivery tube. The delivery tube contains outer layer 14, which is chosen for its biocompatibility and elasticity, and inner layer 15, which is chosen for its ability to provide radial strength and reduce permeation of water. Inner layer 15 can also be chosen for compatibility with the components of the pharmaceutical composition to be delivered using the device. The layers can be laminated polymer layers. In some embodiments, the outer polymer layer is a polyurethane layer and the inner layer is a PET layer.

FIG. 5 is a drawing showing a delivery tube with a flow-stopping removable plug inserted into the delivery tube, in which the flow-stopping removable plug is encased in a heat shrink tubing and the delivery tube, removable plug, and heat shrink tubing are enwrapped by a polymer sheath. The removable plug seals the delivery tube and prevents extrusion of the drug-containing fluid from the device before use. The heat shrink tubing facilitates removal of the removable plug from the delivery tube before use (e.g., after the polymer sheath has been removed). The polymer sheath slows water permeation, reducing or preventing the drug-containing fluid in the delivery tube from drying during extended storage. As shown in FIG. 5, the tail of the polymer sheath can contain a slit to facilitate peeling apart the polymer sheath for removal just prior to use by a patient. After the sheath is peeled off, part of the removable plug (e.g., the removable plug encased in a heat shrink tubing) is exposed. The removable plug can then be pulled out of the delivery tube (e.g., by pulling on the heat shrink tubing that contains the removable plug) to start the flow of the drug-containing fluid.

FIGS. 6A-6D are a series of drawings showing how the polymeric sheath can be removed from the drug delivery device (e.g., peeled off of the removable plug (encased in a heat shrink tubing) and the delivery tube by pulling apart the slit tail) before initial use of the device. A patient or caregiver can grip the ends of the slit tail of the polymer sheath (FIG. 6A) and pull apart the two ends of the slit tail (FIGS. 6B and 6C) until the entire polymer sheath is removed (FIG. 6D), thereby exposing the delivery tube and the heat shrink tubing encasing the removable plug.

FIGS. 7A-7B are a series of drawings showing how the removable plug can be removed from the drug delivery device (e.g., pulled out of the delivery tube by grasping the heat shrink tubing that encases the removable plug) before initial use of the device to start the flow of the drug-containing fluid. The delivery tube can be gently held in one hand (FIG. 7A) and the heat shrink tubing encasing the removable plug can be grasped in the other hand and pulled out of the delivery tube (FIG. 7B) to remove the plug from the delivery tube and prepare the device for use by a patient.

FIG. 8 is a drawing showing an exemplary drug delivery device mounted on a retainer that delivers a drug-containing fluid on the lingual side of a patient’s teeth. The delivery tube wraps around the rear-most tooth of the patient.

FIG. 9 is a drawing showing a close-up view of an exemplary drug delivery device positioned within a retainer pocket.

FIG. 10 is a drawing showing an alternative view of an exemplary drug delivery device positioned in a retainer pocket.

FIG. 11 is a drawing showing an exemplary retainer locating the drug delivery device in the buccal vestibule so as to prevent or reduce interference with speech.

FIG. 12 is a drawing showing a view of an exemplary retainer containing a retainer pocket for holding a drug delivery device from above.

FIGS. 13A-13C are a series of drawings demonstrating how to insert the drug delivery device into the retainer pocket of a retainer (FIGS. 13A and 13B) and how to remove the drug delivery device from the retainer pocket of the retainer (FIG. 13C) when the drug delivery device needs to be replaced. The device can be inserted into the retainer pocket with one hand while the other hand is used to hold the retainer containing the retainer pocket for receiving the drug delivery device.

FIGS. 14A-14B are a series of drawings showing how the retainer-mounted drug delivery device is inserted into the mouth of a patient (FIG. 14A) and the position of the drug delivery device in the mouth once the retainer is properly positioned (FIG. 14B). As shown in FIG. 14A, the drug delivery device can be inserted into the retainer pocket before the retainer is inserted into the mouth.

FIGS. 15A-15C are a series of images showing where the drug delivery device can be positioned in the mouth for optimal comfort with respect to the occlusal surfaces of the rearmost two molars. The drug delivery device can be positioned parallel to the occlusal plane of a molar such as the first or second molar.

FIGS. 16A-16C are a series of drawings showing where the drug delivery device can be positioned in the mouth for optimal comfort. FIG. 16A shows that the device spans only the first and second molars, FIG. 16B shows that the midline of the pump lies on the occlusal plane of the molars, and FIG. 16C shows the optimal positioning range of the drug delivery device anterior and posterior to the rearmost molar.

FIG. 17 is an image showing an adhesive-bonded oral drug delivery device. In this device, both of the housings have rims with a step. The step can serve to increase the adhesive-bonded area, which can prevent failure of an adhesive bond subjected to the force associated with the pressure of a pump (e.g., the force of the propellent chamber in a propellant-driven pump) that is transmitted to a drug reservoir (e.g., a drug chamber containing the drug-containing fluid). Mating flange features in both housings can provide a gap, shown by a hollow arrow, that can be filled with adhesive. Filling the gap with adhesive can hermetically seal the device.

FIG. 18 is a drawing showing the typical dimensions of the flanges (rims) of a propellant housing, such as propellant housing 1 of FIGS. 1 and 2, and their steps, which can be used to increase the adhesive bonded area.

FIG. 19 is a series of drawings showing the typical dimensions of the flanges (rims) of a drug housing, such as drug housing 3 of FIGS. 1 and 2, and their steps, for strengthening the joint, that can optionally be adhesive bonded.

FIG. 20 is a drawing showing an assembled drug delivery device having a metallic housing, the housing having tabs for crimping in which the tabs have already been crimped. Crimping the tabs can strengthen the drug delivery device. It can also form a joint that can be a hermetically sealed joint.

FIG. 21 is a drawing showing an assembled drug delivery device that has tabs for crimping (indicated by an arrow). The tabs have already been crimped in this device.

FIG. 22 is a series of drawings showing a housing of a drug delivery device with tabs for crimping.

FIG. 23 is a drawing showing components of the septum for filling a propellant chamber with propellant (propellant chamber 1, septum eyelet 4, and septum 5).

FIG. 24 is a series of drawings showing the eyelet of a septum for filling the propellant chamber with propellant. An internal rib in the eyelet can increase resistance to needle insertion and removal.

FIG. 25 is a series of drawings showing an elastomeric plug for sealing the drug-containing fluid within the drug chamber.

FIG. 26 is a drawing of an exemplary metal-walled drug housing, showing its dimensions and dimensions of its port used for filling with a drug-containing fluid.

FIG. 27 is a drawing of an exemplary storage case that can stop extrusion of drug-containing fluid from a drug delivery device when the device is in the case and the lid of the storage case is closed, but that allows drug-containing fluid to be extruded from the drug delivery device when the lid is opened. The arrow indicates a holder that can be used to align the device (e.g., a fastener-mounted drug delivery device) in the case. The device contains a diagram that a patient can use for guidance for positioning the device within the storage case.

FIG. 28 is a drawing of an exemplary storage case that can be used to stop extrusion of drug-containing fluid from a drug delivery device when the device is in the case and the lid of the storage case is shut, but that allows drug-containing fluid to be extruded from the drug delivery device when the lid is opened. The arrows indicate the components of the storage case that pinch or kink the delivery tube to stop extrusion of the drug-containing fluid when the storage case is closed.

FIG. 29 is a series of images of the storage case containing a retainer-mounted drug delivery device. The delivery tube of the drug delivery device is positioned such that it is pinched by the components of the case when the storage case is shut.

FIGS. 30A-30C are a series of drawings depicting the insertion of a retainer-mounted drug delivery device into a storage case. A patient can insert the retainer-mounted drug delivery device based on the guide image shown on the bottom interior surface of the storage case (FIG. 30A). The drug delivery device can be inserted into the holder by sliding it into place (FIG. 30B). When the drug delivery device is properly positioned in the storage case, the delivery tube is positioned such that it is pinched by the opposing components on the interior of the case that reversibly compress the tube when the storage case is closed to temporarily stop extrusion of the drug-containing fluid (FIG. 30C, which shows a close up of the drug delivery tube on the elastomeric pad that works with the metal pin in the lid of the storage case to pinch or kink the drug delivery tube when the storage case is closed).

FIG. 31 is an image showing the kinking of the delivery tube when the storage case is closed (part of the storage case is not shown in this image so that the kinking mechanism can be observed).

### DETAILED DESCRIPTION OF THE INVENTION

The devices, compositions, and methods of the invention are useful for continuous or semi-continuous oral delivery of medicaments.

While syringes, drug reservoirs and pumps outside the mouth can be large because space is typically available, space in the mouth for a drug delivery device is limited and is particularly limited when a drug delivery device is so small that it does not interfere with speaking, swallowing, drinking, or eating. Consequently, the delivered drug, its reservoir and its delivery device must occupy a small volume. In the exemplary management of Parkinson’s disease, the concentration of the orally infused LD and/or CD including fluid of the invention can be typically greater than 1 M, such as greater than 1.5 M, 2 M, 2.5 M, 3 M, 3.5 M, 4 M or 4.5 M. These are substantially higher concentrations than the 0.1 M LD concentration of the Duodopa (also known as Duopa™}) gels that are commercially available for jejunal or duodenal infusions. The concentrated drug suspension contained in a drug delivery device of the invention can be viscous, for example its dynamic viscosity at 37° C. can be much greater than 100 cP, such as greater than 10,000 cP, 100,000 cP, or 1,000,000 cP. The suspension can have, for example, viscosity equal to or greater than that of toothpaste, the viscosity being greater than about 20,000 cP, for example greater than 50,000 cP, such as greater than 500,000 cP. The earlier practice of infusion of viscous fluids through long tubings, typically longer than 50 cm, such as those used for nasogastric, jejunal, or duodenal infusions, required that their internal diameter be large and/or that the pumping pressure be high. Furthermore, when the earlier suspensions were infused through the longer tubings, the likelihood of blockage of the flow because of clustering of the suspended LD particles increased and translucent, very fine particle colloids were used to reduce blockage. In contrast, the herein disclosed orally infused, more much concentrated suspensions are typically opaque because they can contain large solid particles scattering visible-wavelength light. The much more concentrated and much more viscous orally infused suspensions can be rich in particle sizes greater than 1 µm, 5 µm, 10 µm, or even 50 µm. The suspensions can be orally infused, for example, through flow restrictors such as orifices in reservoirs that are narrower than 2 mm, 1 mm, or 0.5 mm and/or flow restrictors such as plastic tubings or nozzles that can be shorter than 5 cm, e.g., shorter than 4 cm, 3 cm, 2 cm or 1 cm.

The devices described herein include removable, intraoral drug delivery devices that reside in the mouth and contain a pump, a drug reservoir containing a drug-comprising fluid, and a reversibly compressible delivery tube (e.g., a delivery tube that can be compressed and, after the compression has ended, recovers its shape). The reversibly compressible tube can be multilayered, containing an outer layer that is elastomeric and biocompatible with the inner mucosa (e.g., compatible with living tissue, for example, the material does not cause sensitivity or irritation of the oral mucosa and does not cause systemic toxicity or cytotoxicity) and an inner layer that is compatible with the drug-comprising fluid and that provides radial strength and reduces the permeation of water. The tube can be pinched or kinked to temporarily stop the extrusion of the drug-comprising fluid from the delivery tube when a patient using the device removes the device from the mouth. The invention also features storage cases having a mechanism that pinches or kinks the delivery tube when the storage case is closed, thereby temporarily pausing flow of the drug-comprising fluid. Before the device is first used by a patient, the delivery tube is sealed using a removable plug that is optionally encased in a heat shrink tubing and a polymer sheath, which can be removed when the patient is ready to use the device. To use the device, a patient can insert the device into a pocket attached to a retainer, which holds the device in place on a buccal side of the patient’s teeth on the occlusal plane of a molar and allows the drug delivery tube to wrap around the rear-most molar to deliver the drug-comprising fluid on the lingual side of the teeth. The drug delivery devices may also feature adhesive bonding and/or tabs for crimping to strengthen the device and/or hermetically seal the device. Before the device is used, it can be stored for at least 6 months in a hermetically sealed pouch having an atmosphere that is substantially free of oxygen.

Using the devices and methods of the invention, drugs can be administered intraorally (i.e., onto or near any intraoral salivated or mucous surface, e.g., the lips, cheeks, gums, teeth, tongue, roof of the mouth, hard palate, soft palate, uvula, and glands). The drugs administered intraorally are typically swallowed by the patient, together with the patient’s saliva. The drugs can be diluted by the patient’s saliva and can optionally be partly or fully dissolved in the saliva. The drugs can be absorbed in the patient’s gastrointestinal tract, e.g., in the small intestines or large intestines.

The devices and methods of the invention permit continuous or semi-continuous administration of drugs to subjects in order to produce reliable and steady pharmacokinetic performance are needed to improve efficacy and/or safety of therapies, particularly for drugs with a short half-life (e.g., in the plasma), and/or short persistence of the drug’s physiological effect, and/or a narrow therapeutic window.

### Therapy

The devices and methods of the invention are suitable for the administration of a variety of drugs that have a short half-life and/or a narrow therapeutic range. Complementary drugs may be co-administered or co-infused with these drugs. Such complementary drugs may improve the pharmacokinetics, improve the efficacy, and/or reduce the side effects of the primary drugs.

Gastroparesis, or delayed gastric emptying, is common in people with PD. Drugs for the treatment of gastroparesis may be delivered using the devices and methods of the invention. In one embodiment, drugs for the treatment of gastroparesis are co-administered with the LD or CD, using the drug delivery devices and methods of the invention. In another embodiment, drugs for the treatment of gastroparesis are administered using other methods of drug delivery known in the art (i.e., they are not administered via continuous or frequent intra-oral delivery) while LD or CD are infused intra-orally. Examples of drugs for the treatment of gastroparesis are Metoclopramide, Cisapride, Erythromycin, Domperidone, Sildenafil Citrate, Mirtazapine, Nizatidine, Acotiamide, Ghrelin, Levosulpiride, Tegaserod, Buspirone, Clonidine, Relamorelin, Serotonin 5-HT4 agonists and dopamine D2 or D3 antagonists.

Because administration is into the mouth, it is preferred that the drugs selected for administration are those whose taste is neutral or pleasant, as perceived by a majority of patients. Taste masking or modifying excipients may be added to the formulations of drugs whose taste is unpleasant, as perceived by a majority of patients.

Other drugs that may usefully be delivered in accordance with the invention include methylphenidate, prostaglandins, prostacyclin, treprostinil, beraprost, nimodipine, pyridostigmine or a pyridostigmine prodrug, pilocarpine or a pilocarpine prodrug, levodopa or a levodopa prodrug, baclofen or a baclofen prodrug, furosemide or a furosemide prodrug, and testosterone. Levodopa prodrug formulations of are provided in U.S. Pat. No. 5,607,969, and in patent applications WO 2012/079072 and WO 2013/184646, each incorporated herein by reference. The preferred LD prodrugs for administration into the mouth include highly soluble levodopa amides, levodopa esters, levodopa carboxamides, levodopa sulfonamide, levodopa phosphate prodrugs (e.g., foslevodopa, also known as levodopa 4′-monophosphate, see, e.g., Huters et al., J Org. Chem. 2021 and Rosebraugh et al., Ann Neurol. 90:52-61, 2021), levodopa ethyl ester, levodopa methyl ester, and their salts. An exemplary baclofen prodrug is arbaclofen placarbil. Exemplary pilocarpine prodrugs include alkyl and aralkyl esters of pilocarpic acid, pilocarpic acid diesters, and other pilocarpic acid derivatives, such as those described in Bundgaard et al., J Pharm Sci 75:36-43, 1986, Bundgaard et al., J Pharm Sci 75:775-83, 1986, and U.S. Pat. No. US4742073A, which are incorporated herein by reference. Exemplary furosemide prodrugs include furosemide esters, such as the neutral alkyl ester, alkyl esters containing an amino group, glycolamide esters and O-acyloxymethyl ester described in Mork et al., International Journal of Pharmaceutics 60:163-169, 1990, acyloxymethyl esters of furosemide described in Prandi et al., Farmaco 47:249-63, 1992 and Prandi et al., Farmaco 47:1225-34, 1992, and the furosemide analogs and prodrugs described in International Patent Application Publication No. WO2014039454A2, which are incorporated herein by reference.

Examples of drugs that are often prescribed to be dosed four times per day include:
Amoxicillin — infection
Cephalexin (Keflex) - infection
Chlorpromazine (Thorazine) - neuroleptic for migraine
Diazepam (Valium) - anxiety and sleep
Diclofenac (Voltaren) - arthritis
Diltiazem - calcium channel blocker
Erythromycin - infection
Haloperidol (Haldol) - neuroleptic for migraine
Impramine - psychotropic
Ipratropium (Atrovent) - Anticholinergic
Metoclopramide (Reglan) - gastroesophageal reflux, migraine
Nifedipine - calcium channel blocker
Olanzapine (Zyprexa) - neuroleptic for migraine
Prochlorperazine (Compazine) - neuroleptic for migraine
Promethazine (Phenergan) - neuroleptic for migraine
Salbutamol - asthma
Tetracycline - infection
Theophylline (Theolair) - COPD, asthma
Trazodone — psychotropic
Vaccines---immunotherapy
Zaleplon---insomnia, disordered sleep
Zolpidem---insomnia, disordered sleep

Drugs delivered as solids may be formulated with excipients to increase disintegration or dispersion.

Many types of drugs may be delivered in accordance with the invention. Drugs which may in principle be used for treatment according to the invention are any known drugs, wherein the drugs may be present in the form according to the invention as such, or in the form of the active ingredient, optionally in the form of a pharmaceutically acceptable salt of the active ingredient, or in the form of a prodrug that is rapidly converted to the active drug. Drugs which may be delivered in accordance with the invention include, without limitation, analgesics and anti-inflammatory agents (e.g., aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac), anthelmintics (e.g., albendazole, bephenium hydroxynaphthoate, cambendazole, dichlorophen, ivermectin, mebendazole, oxamniquine, oxfendazole, oxantel embonate, praziquantel, pyrantel embonate, thiabendazole), anti-arrhythmic agents (e.g., amiodarone HCl, disopyramide, flecainide acetate, quinidine sulphate, anti-bacterial agents (e.g., benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim), anti-coagulants (e.g., dicoumarol, dipyridamole, nicoumalone, phenindione), antidepressants (e.g., amoxapine, maprotiline HCl, mianserin HCl, nortriptyline HCl, trazodone HCl, trimipramine maleate), antidiabetics (e.g., acetohexamide, chlorpropamide, glibenclamide, gliclazide, glipizide, tolazamide, tolbutamide), anti-epileptics (e.g., beclamide, carbamazepine, clonazepam, ethotoin, methoin, methsuximide, methylphenobarbitone (methylphenobarbital), oxcarbazepine, paramethadione, phenacemide, phenobarbitone, phenytoin, phensuximide, primidone, sulthiame, valproic acid, topiramate, lamotrigine, gabapentin, levetiracetam, pregabalin), antifungal agents (e.g., amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid), antigout agents (e.g., allopurinol, probenecid, sulphin-pyrazone), antihypertensive agents (e.g., amlodipine, benidipine, darodipine, diltiazem HCl, diazoxide, felodipine, guanabenz acetate, isradipine, minoxidil, nicardipine HCl, nifedipine, nimodipine, phenoxybenzamine HCl, prazosin HCl, reserpine, terazosin HCl), antimalarials (e.g., amodiaquine, chloroquine, chlorproguanil HCl, halofantrine HCl, mefloquine HCl, proguanil HCl, pyrimethamine, quinine sulphate), anti-migraine agents (e.g., dihydroergotamine mesylate, ergotamine tartrate, methysergide maleate, pizotifen maleate, sumatriptan succinate), anti-muscarinic agents (e.g., atropine, benzhexol HCl, biperiden, ethopropazine HCl, hyoscyamine, mepenzolate bromide, oxyphencyclimine HCl, tropicamide), anti-neoplastic agents and immunosuppressants (e.g., aminoglutethimide, amsacrine, azathioprine, busulfan, chlorambucil, cyclosporin, dacarbazine, estramustine, etoposide, lomustine, melphalan, mercaptopurine, methotrexate, mitomycin, mitotane, mitoxantrone (mitozantrone), procarbazine HCl, tamoxifen citrate, testolactone), anti-protozoal agents (e.g., benznidazole, clioquinol, decoquinate, diiodohydroxyquinoline, diloxanide furoate, dinitolmide, furazolidone, metronidazole, nimorazole, nitrofurazone, ornidazole, tinidazole), anti-thyroid agents (e.g., carbimazole, propylthiouracil), anxiolytic, sedatives, hypnotics and neuroleptics (e.g., alprazolam, amobarbital (amylobarbitone), barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, fluanisone, flunitrazepam, fluopromazine, flupentixol (flupenthixol) decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone), β-Blockers (e.g., acebutolol, alprenolol, atenolol, labetalol, metoprolol, nadolol, oxprenolol, pindolol, propranolol), cardiac inotropic agents (e.g., amrinone, digitoxin, digoxin, enoximone, lanatoside C, medigoxin), corticosteroids (e.g., beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone), diuretics (e.g., acetazolamide, amiloride, bendrofluazide, bumetanide, chlorothiazide, chlorthalidone, ethacrynic acid, furosemide (frusemide), metolazone, spironolactone, triamterene), anti-parkinsonian agents (e.g., bromocriptine mesylate, lisuride (lysuride) maleate), gastrointestinal agents (e.g., bisacodyl, cimetidine, cisapride, diphenoxylate HCl, domperidone, famotidine, loperamide, mesalazine, nizatidine, omeprazole, ondansetron HCl, ranitidine HCl, sulphasalazine), histamine H,-receptor antagonists (e.g., acrivastine, astemizole, cinnarizine, cyclizine, cyproheptadine HCl, dimenhydrinate, flunarizine HCl, loratadine, meclozine HCl, oxatomide, terfenadine), lipid regulating agents (e.g., bezafibrate, clofibrate, fenofibrate, gemfibrozil, probucol), nitrates and other anti-anginal agents (e.g., amyl nitrate, glyceryl trinitrate, isosorbide dinitrate, isosorbide mononitrate, pentaerythritol tetranitrate), opioid analgesics (e.g., codeine, dextropropoxyphene, diamorphine, dihydrocodeine, meptazinol, methadone, morphine, nalbuphine, pentazocine), sex hormones (e.g., clomiphene citrate, danazol, ethinyl estradiol, medroxyprogesterone acetate, mestranol, methyltestosterone, norethisterone, norgestrel, estradiol, conjugated oestrogens, progesterone, stanozolol, stilbesterol, testosterone, tibolone), and stimulants (e.g., amphetamine, dexamphetamine, dexfenfluramine, fenfluramine, mazindol).

The above-stated compounds are predominantly stated by their international nonproprietary name (INN) and are known to the person skilled in the art. Further details may be found, for example, by referring to International Nonproprietary Names (INN) for Pharmaceutical Substances, World Health Organization (WHO).

Gastroparesis, delayed or erratic gastric emptying, and other abnormalities or diseases of the stomach, intestine, pylorus, jejunum, duodenum impact the transport of food and medication from the stomach to the duodenum and through the small and large intestines. Such conditions of the GI tract are commonly caused by or associated with various diseases and conditions, including Parkinson’s disease, diabetes, autonomic neuropathy, and cancer treatments. Reduced, delayed, or erratic transport of medication from the stomach to the duodenum and through the small and large intestines decreases the benefits or effectiveness of many drugs, including levodopa. It is for this reason that the Duopa™ (also known as Duodopa™) LD/CD delivery system infuses a LD/CD suspension into the jejunum or duodenum, even though intrajejunal delivery requires surgical implantation of a PEG tube and suffers from a high rate of PEG tube related complications. The inventors discovered that the oral intake of an aqueous solution of L-DOPA and carbidopa at frequency of about 6 — 12 times/hour also stabilizes the plasma concentration of L-DOPA and reduces by about 43% the OFF time of PD patients. Without limiting the scope of this invention by a theory or model, the inventors have observed that the reported gastric delay of drugs does not necessarily apply when the drugs are continuously orally infused and are dissolved. Thus, it can be advantageous to infuse into the mouths of patients a suspension or paste including solid drug particles at a rate that equals or is slower than the rate of dissolution of the solid drug particles in body fluids secreted in the mouth, such that the drug passing through the esophagus to the stomach is already substantially dissolved, such that the remaining solid drug particles are substantially dissolved in fluid secreted in the stomach, and/or such that the still remaining drug particles are substantially dissolved in fluid secreted in the duodenum, then, if solid drug particles still remain, these are substantially dissolved in fluids secreted in the jejunum, then if still present, substantially dissolved in fluids secreted in the ileum, and eventually if still present, substantially dissolved in fluids secreted in the colon. The secreted body fluid in which the solid drug may dissolve can be, for example, saliva secreted in the mouth (e.g., by the submandibular and parotid glands) mostly in the awake hours. In healthy persons the rate of secretion can be between about 50 mL/hour and about 100 mL/hour. Considering that the solubility of LD can be about 50 mg/mL and considering that even if a patient would require as much as 200 mg LD per hour, as little as about 4 mL/hour of saliva could dissolve the orally delivered solid LD. The drug could not only be dissolved, but its solution might be diluted before reaching the stomach even in patients (e.g., patients with PD or xerostomia) secreting less saliva than healthy people. For rapid dissolution in saliva it could be advantageous to disperse the drug particles (e.g., by administering their surfactant-including suspension) where the size of the drug particles could be small (e.g., typically less than about 100 µm in average diameter, such as less than 50 µm in average diameter, such as less than 20 µm in average diameter, such as less than10 µm in average diameter).

Other drugs, such as baclofen or pyridostigmine, that are administered in lesser daily amounts than LD could be adsorbed on small particles of a solid excipient, such as an amino acid like tyrosine. For continuous oral delivery, the paste of the drug-containing excipient could be extruded into the mouth where the excreted saliva would dissolve the sorbed drug as well as any solid drug particles, if present.

The drug delivery devices can be used to orally administer drugs to patients in therapeutically effective amounts. For example, an amount is administered which prevents, delays, reduces, or eliminates the symptoms of a disease, such as PD, mucositis, bacterial infections, viral infection, fungal infection, parasite caused disease, cancer, pain, organ transplantation, disordered sleep, epilepsy and seizures, anxiety, mood disorders, post-traumatic stress disorder, cancer, arrhythmia, hypertension, heart failure, spasticity, diabetic nephropathy, and allergy. They can also be used to manage allergies, for example, to peanuts, e.g. by delivering agents, such as fractions or whole allergy causing antigens, for sublingual immunotherapy such that the delivered agents contact a mucous membrane or tissue of the mouth. Using the drug delivery devices, a drug appropriate for the treatment of a given disease to be treated can be formulated and administered using the methods, compositions, and devices described herein.

Many drugs with narrow therapeutic indices benefit from drug delivery devices and methods that result in small fluctuation indices. For example, Table 1 summarizes the fluctuation indices of extended release tablet formulations of anti-epileptic drugs reported in various studies (from “Extended-release antiepileptic drugs: A comparison of pharmacokinetic parameters relative to original immediate-release formulations”, Ilo E. Leppik and Collin A. Hovinga, Epilepsia, 54(1):28-35, 2013).

TABLE 1

Fluctuation indices of anti-epileptic drug extended release tablets.

| Drug | Fluctuation Index (SD) |
|---|---|
| Carbamazepine | 0.31 (0.1) |
| | 0.26 (0.1) |
| | 0.47 |
| | 0.49 |
| Divalproate sodium | 0.39 (0.15) |
| | 0.67 (0.16) |
| | 0.34 (0.15) |
| | 0.67 (0.17) |
| | 0.59 (0.27) |
| | 0.46 (0.16) |
| | 0.71 (0.20) |
| Lamotrigine | 0.341 |
| | 0.817 |
| | 0.209 |
| | 0.545 |
| | 0.986 |
| | 0.318 |
| Oxcarbazepine | 0.39 (0.08) |
| | 0.54 (0.09) |
| Levetiracetam | 1.19 |
| | 1.27 |

The invention includes a method of treating a disease or medical condition using any of the devices, drugs, formulations, and methods disclosed herein, wherein the fluctuation index is less than or equal to 2.0, 1.5, 1.0, 0.75, 0.50, 0.25, or 0.15. For example, the disease or medical condition to be treated may be Parkinson’s disease, bacterial infection, viral infection, fungal infection, cancer, pain, organ transplantation, disordered sleep, epilepsy and seizures, anxiety, mood disorders, post-traumatic stress disorder, cancer, arrhythmia, hypertension, heart failure, spasticity, dementia, diabetic nephropathy, xerostomia, and dementia.

Drug dosages administered using the methods of the invention may be higher or lower than those administered using traditional, infrequent dosing regimens. A lower daily dose is possible without loss of efficacy when continuous or semi-continuous administration reduces troughs in the drug’s steady state circulating plasma concentration, enabling the drug’s plasma concentration to remain above the minimum effective plasma concentration without the need for high peak concentrations. A higher daily dose is possible without increased side effects when continuous or semi-continuous administration reduces peaks in the drug’s steady state circulating plasma concentration, enabling an increase in the drug’s average plasma concentration without the need for high peak concentrations.

The devices and methods of the invention provide a dosing regimen having an improved safety profile as adverse events associated with peak plasma concentrations (i.e., a Cₘₐₓ characteristic of oral unit dosage forms) are eliminated. Thus, the devices and methods of the invention can be used to deliver drugs having a narrow therapeutic window in the patient population being treated (i.e., patients refractory to standard therapeutic regimens). Details provided below for the treatment of PD can be applicable to the formulation and administration of drugs for the treatment of other diseases.

For the treatment of PD, typical administered dose ranges are from about 20 µmole/kg to about 200 µmole/kg of LD or LD prodrug per day. The typical daily dose of the optionally co-administered DDC inhibitor is between about 5 µmole/kg and about 50 µmole/kg. For example, the typical daily dose for a patient weighing 75 kg is from about 1.5 millimoles to about 15 millimoles of LD or LD prodrug. Optionally, a molar amount of a DDC inhibitor between about 10% and about 40% of the molar amount of the LD or LD prodrug, for example between 15% and 30%, may be added. Optionally, COMT inhibitors such as entacapone, tolcapone, or opicapone may be administered using the devices of the invention. Exemplary LD prodrug formulations of the prior art are provided in U.S. Pat. No. 5,607,969, and in patent applications WO 2012/079072 and WO 2013/184646, each incorporated herein by reference. The preferred prodrugs for administration into the mouth include highly soluble levodopa amides, levodopa esters, levodopa carboxamides, levodopa sulfonamide, levodopa phosphate prodrugs (e.g., foslevodopa, also known as levodopa 4′-monophosphate, see, e.g., Huters et al., J Org. Chem. 2021 and Rosebraugh et al., Ann Neurol. 90:52-61, 2021), levodopa ethyl ester, levodopa methyl ester, and their salts, which can be rapidly hydrolyzed in the body, typically in an enzyme catalyzed reaction, to form LD, yet can be stored at least for the duration of the intended administration period, for example at least 8 hours, 16 hours, 24 hours, 48 hours, 72 hours, in a reservoir of the drug delivery device. Additional examples of levodopa prodrugs, including LD ester prodrugs, LD amide prodrugs, LD dimeric amide prodrugs, carrier-mediated prodrugs, peptide transport-mediated prodrugs, and cyclic prodrugs, are described in Haddad et al., Molecules 23:40, 2018, which is incorporated herein by reference.

Preferred modes of administration of the drug-including solid or fluid are via drug delivery devices that are removably secured in the mouth, and which administer the drug into the mouth for a period of at least 4 hours. The drug may be administered at a variable rate, although constant rate administration is preferred. Administration is preferably continuous or semi-continuous.

The administration into the mouth can be for 24 hours daily or it can be limited to the awake period, typically about 16 hours. When limited to the awake period it can be advantageous to administer a morning bolus to more rapidly raise the plasma concentration of the LD than a constant rate administration would. The morning bolus can be delivered, for example, through an orally taken pill or pills of LD and a DDC inhibitor or it can be through administration of a solid or fluid drug into the mouth using the drug devices of the invention. Alternatively, the exterior of the drug delivery device may include a drug, such that a bolus of the drug is delivered into the mouth when the device is first inserted into the mouth.

The invention includes methods of administering into the mouth one or more drugs (e.g., LD and CD) from one or more drug reservoirs residing in the cavity of the mouth including a total volume of 0.1 — 10 mL of drugs (e.g., 0.1-1.0 mL, 1.0-2.0 mL, 2.0-3.0 mL, 3.0-4.0 mL, 4.0-5.0 mL). The invention includes methods of administering the one or more drugs (in either solid or fluid form) at a rate in the range of 0.03 - 1.25 mL/hour (e.g., 0.03 — 0.10 mL/hour, 0.10-0.20 mL/hour, 0.20-0.30 mL/hour, 0.30-0.40 mL/hour, 0.40-0.50 mL/hour, 0.50-0.60 mL/hour, 0.60-0.70 mL/hour, 0.70-0.80 mL/hour, 0.80-0.90 mL/hour, 0.90-1.0 mL/hour, 1.0-1.1 mL/hour, or 1.1-1.25 mL/hour). The invention includes methods of administering the one or more drugs at an average rate of less than 1 mg per hour, 1-10 mg per hour, 10 — 25 mg per hour, 25 — 50 mg per hour, 50 — 75 mg per hour, 75 - 100 mg per hour, 100 — 125 mg per hour, or greater than 125 mg per hour. The invention includes methods of administering one or more drugs via continuous and/or semi-continuous administration. In a preferred embodiment, the method includes holding the average administration rate constant or near constant for a period of 4, 8, 12, 16, or 24 hours during the day. For example, the volume administered every hour may vary from the average hourly administration rate during the infusion period by less than ±10% or ±20% per hour, or by ±10% or ±20% per 15-minute period. The invention includes methods of administering one or more drugs into the mouth using any of the drug delivery devices described herein.

Continuous or semi-continuous administration using the drug delivery devices and formulations of the invention can reduce concentration fluctuations of the therapeutic drug in body fluid, for example in blood, plasma or serum. It can provide, for example, a plasma concentration profile where the difference between peak concentrations and nadir concentrations of the therapeutic drug is less than ±70% of the average concentration through a period in which the drug is administered, for example it can be less than ±50%, less than ±30%, less than ±20%, or less than ±10% of the time averaged concentration over a period of greater than or equal to 4 hours (e.g., 8, 12, 16, or 24 hours).

The invention features a method of treating a disease in a patient, the method including: (a) inserting a drug delivery device into the patient’s mouth; (b) starting a drug administration from the device; (c) administering into the patient’s mouth one or more drugs, using continuous or semi-continuous administration, for a period of 4 hours to 7 days at an hourly rate in the range of 0.015 — 1.25 mL/hour or 1-125 mg/hour; and (d) removing the drug delivery device from the mouth; wherein the drug delivery device includes a drug reservoir of 0.1-5 mL volume (e.g., 0.1-1 mL, 0.5-3 mL, or 3-5 mL), and the reservoir includes a solid or fluid including a drug. Optionally, the method may also include the step of: (e) stopping the drug delivery from the device. The invention further includes a method wherein steps a, b, c, d and e are performed at least twice over a period of 4 hours to 7 days. The drug may include a total of greater than 1 millimole of LD.

The invention features a method of treating a disease in a patient, the method including: (a) inserting a drug delivery device into the patient’s mouth; (b) starting a drug administration from the device; (c) administering into the patient’s mouth one or more drugs, using continuous or semi-continuous administration, for a period of 4 hours to 7 days at an hourly rate in the range of 0.015 — 1.25 mL/hour or 1-125 mg/hour; and (d) removing the drug delivery device from the mouth, wherein: (1) the drug delivery device includes a reservoir of 0.1-5 mL volume (e.g., 0.1-1 mL, 0.5-3 mL, or 3-5 mL), and the reservoir includes a solid or fluid including a drug, and (2) steps a, b, c, and d are performed at least twice over a period of 4 hours to 7 days. The drug may include a total of greater than 1 millimole of LD.

The methods of the invention can further include treating Parkinson’s disease in a patient (including in patients with scores of 4 and 5 on the Hoehn and Yahr scale), the method including: (a) removably inserting a drug delivery device of the invention into the patient’s mouth, the drug delivery device including a reservoir of 0.1-5 mL volume (e.g., 0.1-1 mL, 0.5-3 mL, or 3-5 mL), and the reservoir including a solid or fluid including a total of greater than 1 millimole of LD; (b) administering into the patient’s mouth the solid or fluid for a period of at least 8 hours at an hourly rate in the range of 0.03 — 1.25 mL/hour or 30 - 150 mg/hour, such that a circulating plasma LD concentration greater than 400 ng/mL and less than 7,500 ng/mL is continuously maintained for a period of at least 8 hours during the administration; and (c) removing the drug delivery device from the patient’s mouth. In certain embodiments, the LD suspension is administered into the mouth at such a rate that a circulating plasma LD concentration greater than 800 ng/mL, 1,200 ng/mL, 1,600 ng/mL, or 2,000 ng/mL (e.g., from 800 to 1,500, from 1,000 to 2,000, from 1,600 to 2,500, or from 1,500 to 3,000 ng/mL, depending upon the condition of the patient) is continuously maintained for a period of at least 2 hours, 3 hours, 4 hours, 8 hours, 16 hours, or 24 hours during the administration. In particular embodiments, the LD suspension is administered into the mouth at such a rate that a circulating plasma LD concentration greater than 400 ng/mL, 800 ng/mL, 1,200 ng/mL, 1,600 ng/mL, or 2,000 is achieved within 60 minutes of the initiation of the infusion. The LD suspension can be administered into the mouth at such a rate that a circulating plasma LD concentration less than 7,500 ng/mL, 5,000 ng/mL, 3,500 ng/mL, 3,000 ng/mL, 2,500 ng/mL, or 2,000 ng/mL is continuously maintained for a period of at least 8 hours during the administration. In particular embodiments, the patient receives an average daily dose of less than 10 mL, 7.5 mL, 5 mL, 3 mL, or 2 mL of the LD suspension. The LD suspension can be administered into the mouth at such a rate that the circulating LD plasma concentration varies by less than ±20%, ±15%, or ±10% from its mean for a period of at least 1 hour, 2 hours, 3 hours, or 4 hours.

The method can further include the co-administration of an effective amount of a DDC inhibitor such as benserazide, carbidopa or carbidopa prodrug. Carbidopa can be co-administered as a solid, suspension or emulsion, or as a solution of one of its highly water-soluble prodrug salts, exemplified by carbidopa ethyl ester hydrochloride, by carbidopa methyl ester hydrochloride, by carbidopa amide hydrochloride, or by carbidopa phosphate prodrugs (e.g., foscarbidopa, also known as carbidopa 4′-monophosphate, see, e.g., Huters et al., J Org. Chem. 2021 and Rosebraugh et al., Ann Neurol. 90:52-61, 2021). The molar amount of the co-administered DDC inhibitor can be between one-tenth and one-half of the molar amount of LD, preferably about ¼^{th} ± ⅛^{th} of the molar amount of LD. Preparations of the carbidopa prodrugs, recognized to be LD decarboxylase inhibitors, are described, for example, in U.S. Pat. Nos. 3,895,052 and 7,101,912, and Pat. Publication Nos. DE2062285A and FR2052983A1. In one particular embodiment, a LD suspension includes a greater than 0.5 M LD (e.g., 0.5 ± 0.1, 0.6 ± 0.1, 0.7 ± 0.1, 0.8 ± 0.2, 1.0 ± 0.3, 1.5 ± 0.5, 2.0 ± 0.5, 0.6 ± 0.3, 0.75 ± 0.25, 1.0 ± 0.5, 1.5 ± 0.5, 2.0 ± 0.5, 2.5 ± 0.5, 3.0 ± 0.5, 3.5 ± 0.5, greater than 1.5, greater than 2, greater than 2.5, or greater than 3.5 moles per liter). In particular embodiments, the LD and the DDC inhibitor are co-administered separately, or are contained in a single solid or fluid and administered into the patient.

The method can alleviate a motor or non-motor complication in a patient afflicted with Parkinson’s disease, such as tremor, akinesia, bradykinesia, dyskinesia, dystonia, cognitive impairment, and disordered sleep.

### Drug Delivery Devices

The drug delivery devices of the present invention are designed to address the requirements for a device that is inserted into the mouth by the patient or caregiver, and that resides in the mouth while it is administering drug, and that can be removed from the mouth by the patient or caregiver. Preferred drug delivery devices include drug reservoirs.

The drug delivery devices typically have a total volume of less than about 10 mL, and preferably less than 7.5, 5.0, or 3.0 mL. Preferred volumes for the drug delivery devices are 0.5-3.0 mL, to minimize interference with the patient’s mastication, swallowing and speech.

To prevent their being accidentally swallowed or aspirated into the trachea, the drug delivery devices of the invention are either secured in the mouth or are of a shape and size that cannot be swallowed or aspirated into the trachea. They may be secured to any interior surface of the mouth, such as one or more teeth, the roof of the mouth, the gums, the lips or the cheek within the mouth of the patient. In order to obtain a secure and comfortable fit, the devices may be molded to fit on or attach to a surface within the mouth of a patient, such as the teeth or the roof of the mouth, or they may conform to at least one cheek. In some embodiments, the drug delivery devices are secured such that they are positioned on the teeth, on a cheek, between the gums and the cheek, between the gums and the lips, or at the roof of the mouth. Alternatively, the drug delivery device includes a shape and size that cannot be swallowed. Examples are a curved, elongated shape of greater than 4 cm length in its curved form (e.g., greater than 5, 6 or 7 cm) that can be placed between the gums and the cheek and lips; or drug delivery devices positioned adjacent to both cheeks and connected with a bridge, optionally forming fluidic contacts with both the left and the right parts.

Although the housing of the typical drug delivery device can be a strong material such as a metal or a ceramic, the housing may include in some embodiments a rigid plastic comprising a strong polymer, such as a polyimide. The plastic may optionally be fiber reinforced, i.e., it may be reinforced, for example, by carbon, metal, or glass fibers. The metallic housing can be a stainless steel such as an austenitic stainless steel, or it can be titanium or an alloy of titanium.

The drug delivery devices may be attached to the teeth or other interior surfaces of the mouth by a fastener. The fastener, the one or more pumps, and the one or more drug reservoirs may include a single unit or they may include separate components, with the fastener remaining in the mouth when the one or more pumps or one or more reservoirs are removed. In one embodiment, a pump and a drug reservoir include a single removable component that can be attached to the fastener. Drug can be delivered into the mouth via one or more flow restrictors and/or a delivery tube. In another embodiment, a disposable pump and drug reservoir can be inserted into a reusable housing attached to the fastener. The fastener, one or more drug pumps and one or more drug reservoirs may be removably attached to each other using magnets, clips, clasps, clamps, flanges, latches, retaining rings, snap fasteners, screw mounts, or other attachment mechanisms known in the art. In preferred embodiments, the fastener includes a plastic retainer that can be translucent or transparent. It can be a partial retainer on one side of the mouth (e.g., attached to 2, 3, 4, or 5 teeth).

In some embodiments, the pump and/or drug reservoir is secured to either the upper or lower teeth using a transparent retainer. One, two or more pumps and/or one or more drug reservoirs can be secured on the buccal side of the transparent retainer. One, two, or more drug pumps and/or drug reservoirs may be secured unilaterally, on either the right or left sides, positioned in the buccal vestibule or, alternatively, on the lingual side of the teeth. The drug pump and reservoir can be removably inserted in a plastic receptacle that is integrated in the plastic retainer. Drug can be delivered into the mouth via a delivery tube that does not substantially restrict its flux, the flow being predominantly controlled by the narrower internal diameter flow restrictor or restrictors. The delivery tube can serve to carry the drug from the buccal to the lingual side of the teeth, where the drug may be more readily swallowed. The tube may be attached to the pump or it can be molded into the reusable retainer.

For delivery of some drugs, such as LD or CD, it can be desirable to administer the drug-including solid or fluid on the lingual side of the teeth, rather than on the buccal side of the teeth, in order to minimize the residence time of the drug in the mouth, thereby avoiding potential accumulation of the drug in the buccal vestibule and minimizing potentially irritating exposure of the buccal tissue to the drug. In a preferred embodiment, pump includes a delivery tube to transport the drug-including fluid into the mouth. The delivery tube carries the drug-including fluid from the drug reservoir located on the buccal side of the teeth to the lingual side of the teeth. The delivery tube can, for example, pass behind the rear molars, above the mandibular arch, so that it does not cross the biting surface of the teeth.

The fastener or its components, such as the housings, may be manufactured using methods known in the art, such as thermoforming, injection molding, pressure molding, and laminating.

The drug delivery device may be a single unit, or it may have two, three, four, five or more components. The drug delivery device may have one, two, three, four, five or more drug reservoirs in which the solid or fluid drug formulation is contained. These one or more reservoirs may form a single component, or they may form multiple components.

The drug delivery devices may be reusable, disposable, or they may have one or more reusable components and one or more disposable components. In a preferred embodiment, the fastener is reusable, and may be reused for a period of equal to or greater than 7, 30, 60 or 180 days, or one year or two years. In another preferred embodiment, the one or more drug reservoirs are single use, disposable components. The pump is typically disposable, but it may be reusable. The flow restrictor is typically single use meaning disposable, but it can be reusable in some embodiments.

The drug reservoir is typically single use, but it may be refillable with a solid or fluid drug formulation. In a preferred embodiment, the drug reservoir is a single use disposable. The drug reservoir may be filled by the user, a caregiver, or a pharmacist. In preferred embodiments, the drug reservoir is prefilled.

The drug delivery device may further include one, two, three, four or more orifices for releasing the drug from the device into the mouth.

Durations of administration from a single drug delivery device or drug reservoir typically exceed 4, 8, 12, or 16 hours per day, up to and including 24 hours per day. Administration can also take place over multiple days from a single device or drug reservoir, e.g., administration of a drug for 2 or more days, 4 or more days, or 7 or more days. The devices can be designed such that they can be worn when the patient is awake or asleep.

It is desirable that the patient be able to temporarily remove the drug delivery device from the mouth, for example, to eat meals, brush teeth, or at times when the patient does not want or need the medication (e.g., at night). Consequently, the drug delivery devices and/or some of its components (such as the pump and/or the drug reservoirs) can be temporarily removable. It is, however, acceptable for some components, such as the fastener, to remain in the mouth if these do not interfere with the patient’s activities. For example, a band, a fastener cemented or glued to one or more teeth, a retainer, or a muco-adhesive patch adhered to the oral mucosa, and which holds the pump and/or drug reservoir in place, may remain in the mouth when the pump and/or the drug reservoir are removed.

It is desirable that the drug delivery device include an indicator of: the quantity remaining of one or more drugs; the infusion time remaining until empty; and/or that one or more of the drug reservoirs is empty and should be replaced.

The drug delivery devices can be configured and arranged to administer one or more solid or fluid drug formulations from one or more drug reservoirs including a total volume of 0.1 — 10 mL of drugs, e.g., 0.1-1.0, 1.0-2.0, 2.0-3.0, 3.0-4.0, 4.0-5.0, 5.0-6.0, 6.0-7.0, 7.0-8.0, 8.0-9.0, or 9.0-10 mL. They are configured and arranged to administer the one or more solid or fluid drug formulations at a rate in the range of 0.03 — 1.25 mL/hour, e.g., 0.03 — 0.10, 0.10-0.20, 0.20-0.30, 0.30-0.40, 0.40-0.50, 0.50-0.60, 0.60-0.70, 0.70-0.80, 0.80-0.90, 0.90-1.0, 1.0-1.1, or 1.1-1.25 mL/hour. In some embodiments, they are configured and arranged to administer the drug, (i.e., the active pharmaceutical ingredient) at an average rate of 0.01 — 1 mg per hour, 1 — 10 mg per hour, 10 — 100 mg per hour, or greater than 100 mg per hour. In other embodiments, the drug product (i.e., the active pharmaceutical ingredient plus excipients) is delivered at an average rate of 0.01 — 1 mg per hour, 1 — 10 mg per hour, 10 — 100 mg per hour or greater than 100 mg per hour.

In preferred embodiments, the drug delivery device administers one or more solid or fluid drug formulations via continuous and/or frequent administration, e.g., infusion. In a preferred embodiment, the solid or fluid drug administration rate is held constant or near constant for a period of 4, 8, 12, 16 or 24 hours during the day. For example, the administered volume may vary by less than ±10% or ± 20% per hour, or by ±10% or ± 20% per 15-minute period, over a period of 4, 8, 12, 16 or 24 hours. In another embodiment, the solid or fluid drug administration rate is held about constant during the awake hours of the day. In another embodiment, the solid or fluid drug formulation administration rate is held about constant during the asleep hours. In another embodiment, the solid or fluid drug formulation administration rate is held about constant during the awake hours of the day, except for the delivery of a bolus at about the time of waking. In one embodiment, the administration rate can be set prior to insertion in the mouth by the patient or by the caregiver. In another embodiment, the administration is semi-continuous and the period between the infusions is less than the biological half-life of the drug t_{½}; for example it can be less than one half of t_{½}, less than ⅓rd of t_{½}, or less than ¼ of t_{½}, or less than ⅒th of t_{½}.

### Positioning the Drug Delivery Device on a Fastener

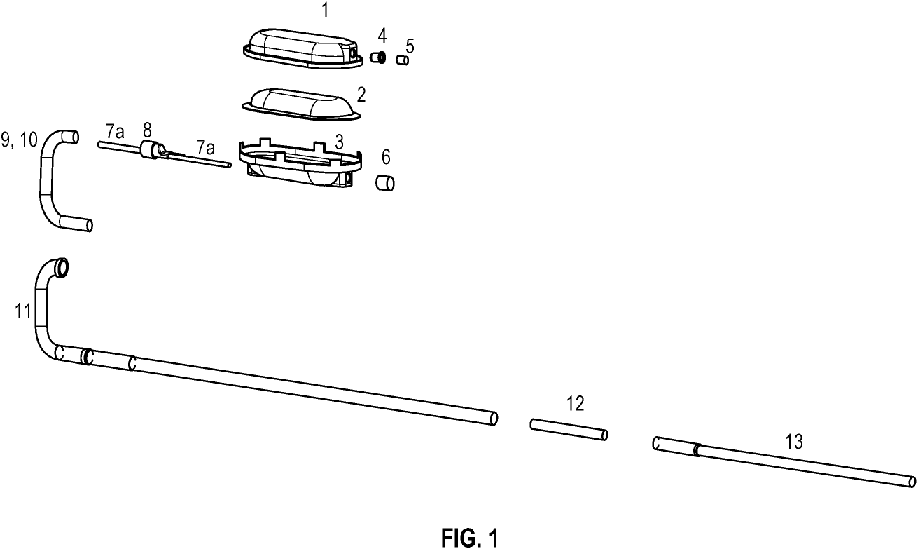

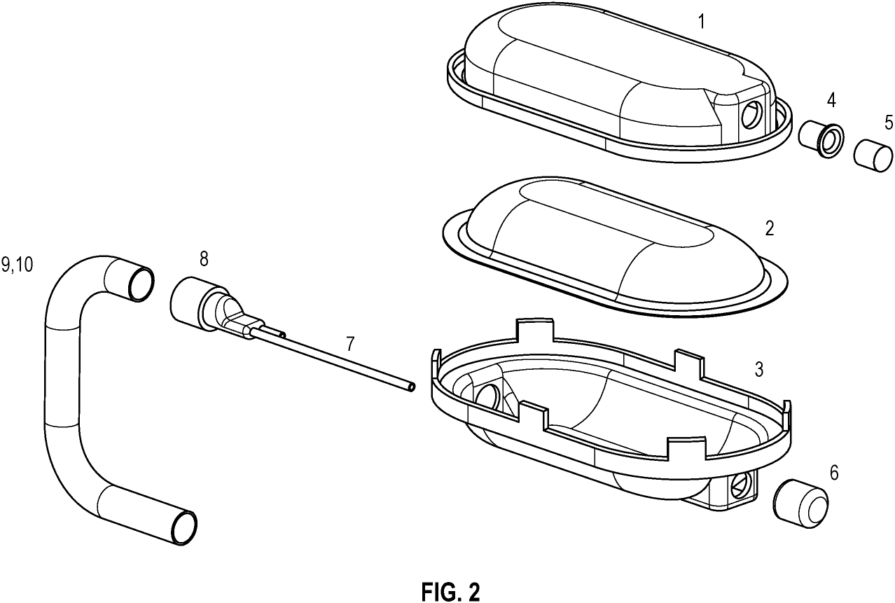

In some embodiments, the drug delivery device is secured to the teeth (e.g., the upper teeth) using a fastener, such as a retainer. The fastener can be used to secure the drug reservoir, the pump, or an assembly containing both components (e.g., an assembly containing both a pump and a drug reservoir or drug chamber, such as the assembly shown in FIGS. 1 and 2) to the teeth of a patient and can be made of a plastic, such as an acrylate-containing polymer or an olefin-containing polymer. For example, the fastener may be made of a thermoplastic polyolefin, such as polypropylene. The plastic may be an Essix® PLUS™ plastic, an Essix A+® plastic, an Essix C+® plastic, an Essix ACE® plastic, an Essix® dual laminate plastic, an Essix® nightguard laminate plastic, an Essix® sports mouthguard material, an Essix® laminated sports mouthguard material, an Essix® bleach tray and model duplication material, an Essix Tray Rite® plastic, a Dreve Drufosoft® Pro plastic, a Dreve Kombiplast plastic, a Dreve Biolon plastic, or a Dreve Drufosoft® sports mouthguard material. The fastener can include a receptacle, meaning a pocket, for holding the drug delivery device, which can be fabricated as an integral part of the fastener, for example, by molding, and it can prevent accidental removal of the device during its handling. In addition, the fastener can be personalized to a patient’s dental anatomy for comfortable wear and for absence of interference with speech or drinking. The fastener can be positioned near the roof of the mouth with its pocket holding the drug delivery device on the buccal side of the teeth, positioning the device in the cheek pocket for comfort. The pocket can also prevent dislodging of the device during insertion of the fastener or wear, and can position the device such that it is in the proper orientation for administration of a drug-containing fluid into the mouth. In some embodiments, the pocket is designed with a feature that maintains an interference fit (also known as a friction fit) with the drug delivery device by way of friction to prevent the device from becoming dislodged. For example, the interference fit may be produced by a tight fit between the mating parts (e.g., the pocket of the fastener and the housing of the drug delivery device) that produces a joint that is held together by friction after the drug delivery device is pushed into the pocket of the fastener. Alternatively, the drug delivery device can be reversibly secured in the pocket with a detent, groove, snap, or lock. The drug delivery device can be positioned within the pocket such that the outlet of the delivery tube, also termed outlet tube, through which the drug-containing fluid (e.g., drug-containing suspension, emulsion, or paste) leaves the assembly is on the lingual side of the teeth, meaning that drug-containing fluid from the device residing in the cheek pocket, or buccal pocket, is delivered to the saliva-rich lingual side of the teeth, near the salivary glands. In a preferred embodiment, the pocket of the fastener is located on the buccal side of the upper molars and holds the drug delivery device such that the delivery tube of the drug delivery device wraps around the rear-most tooth of the patient and delivers drug on the lingual side of the teeth. During testing, it was found that coverage of the soft palate affected comfort and speech, so to improve comfort and minimize the disruption of speech, the fastener (e.g., a retainer) can be positioned such that it spans approximately halfway on the hard and soft palates (e.g., approximately half of the hard palate and half of the soft palate is covered by the fastener). The hard palate is only partially covered by the fastener to ensure that speech is not affected, and the soft palate is only partially covered by the fastener to prevent discomfort and/or gag reflex.

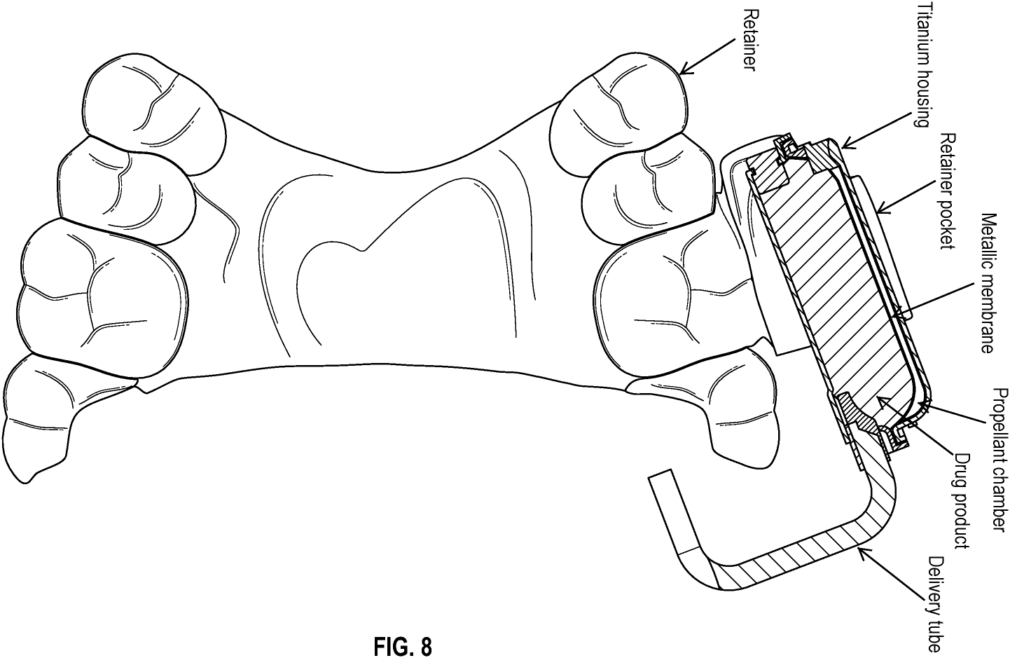

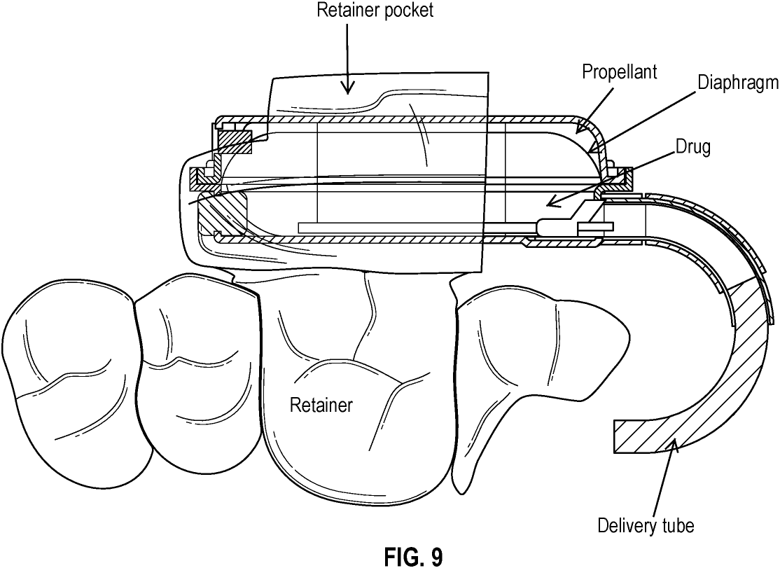

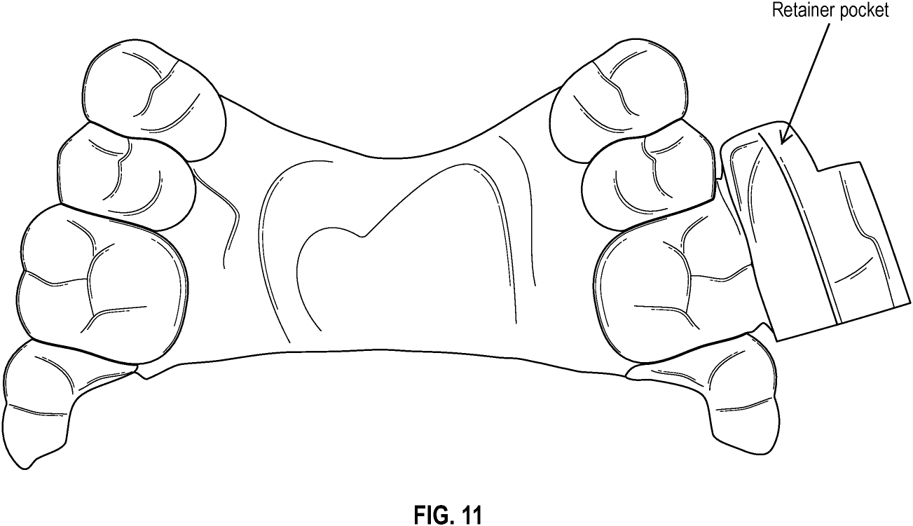

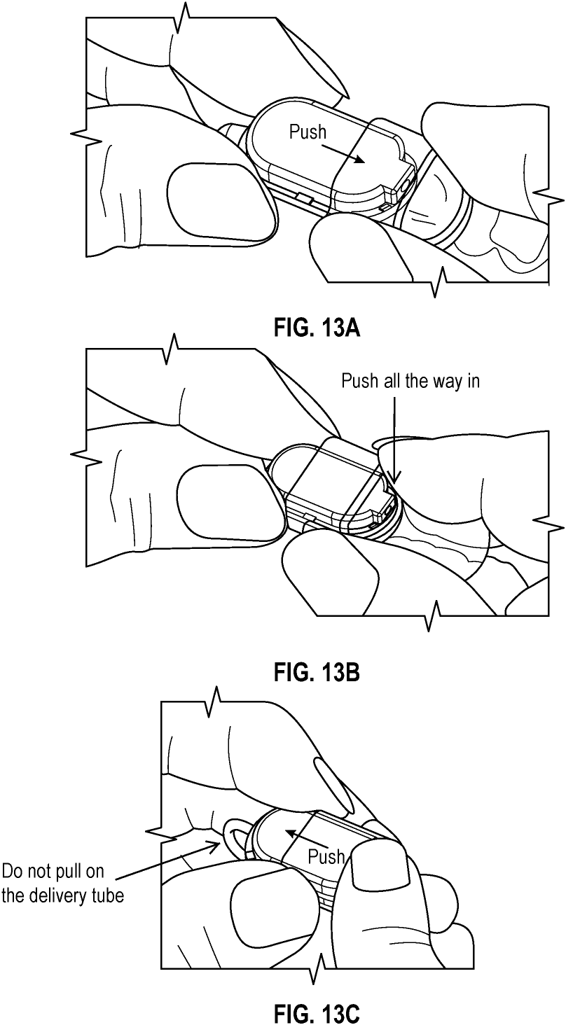

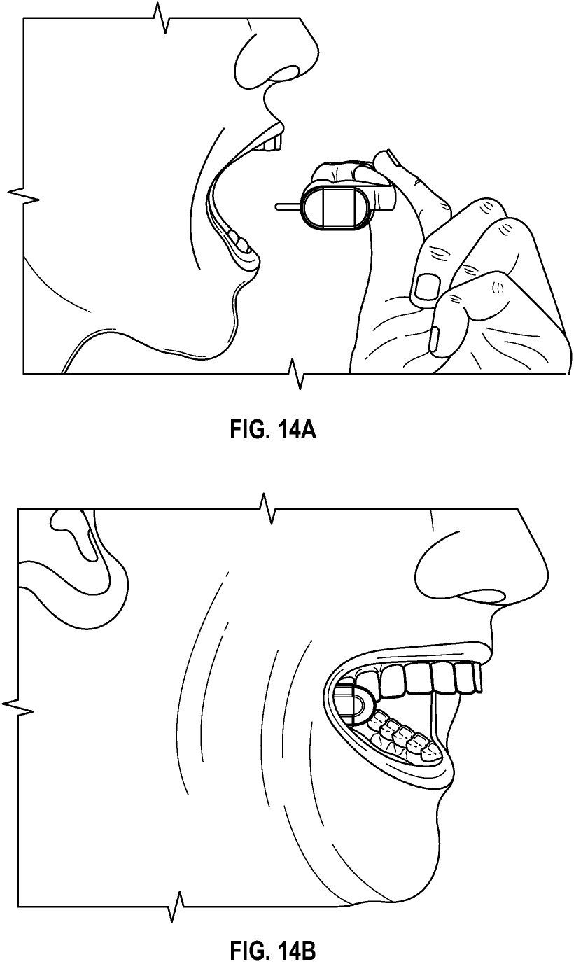

An exemplary fastener that is a retainer is shown in FIGS. 8-12. The retainer can be made of a plastic, such as an acrylate-containing polymer. In some embodiments, the retainer is made of Essix® PLUS™ plastic. As shown in FIGS. 8-12, the retainer includes pocket for holding the drug delivery device on the buccal side of the teeth (e.g., next to the upper molars) and the retainer pocket is an integral part of the retainer. As shown in FIGS. 8 and 9, the retainer pocket positions the drug delivery device such that the delivery tube wraps around the rear-most molar of the patient and the outlet of the drug delivery tube releases drug on the lingual side of the teeth. FIGS. 13A-13B show how the drug delivery device can be inserted into the retainer pocket (e.g., by pushing the drug delivery device all the way into the pocket so it is secured in the proper position for administration). The drug delivery device can be removed from the retainer when needed (e.g., to replace a drug delivery device that has been used continuously for 4 or more hours with a fresh drug delivery device, e.g., when more than half of the drug-containing fluid in the first drug delivery device has been delivered). FIG. 13C shows that the drug delivery device can be removed by pushing the device out of the retainer pocket. FIGS. 14A-14B show how a retainer-mounted drug delivery device can be inserted into the mouth (FIG. 14A) and where the retainer-mounted drug delivery device resides once the retainer has been inserted (FIG. 14B).

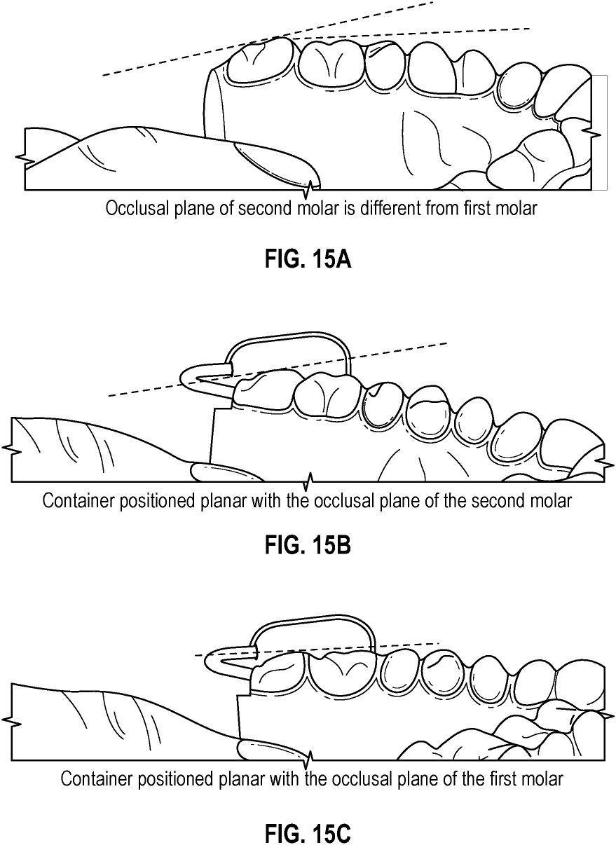

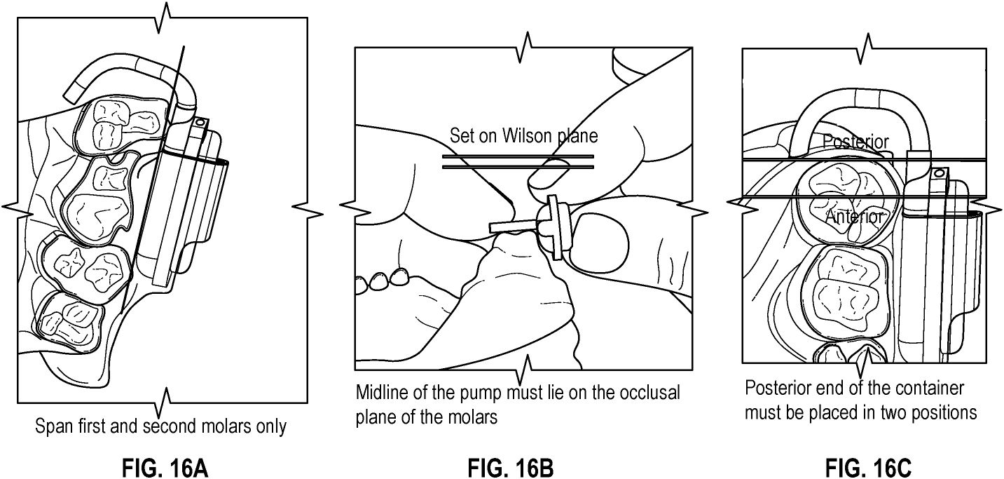

The drug delivery device can be positioned, as shown in FIGS. 15A-15C, coplanar with the occlusal plane of a molar, such as the second molar or the first molar. The retainer can be worn over the bicuspids and molars and can position the drug delivery device such that it spans only the first and second molars, with its midline on the occlusal plane of the molars (e.g., the occlusal plane of the first or second molar), and its posterior end between the midline and the posterior of the rear-most (first or second) molar, as shown in FIG. 16C. The drug delivery device can also be positioned within the fastener (e.g., in the retainer pocket) such that it is set on the Wilson plane. This set of constraints ensures that the device is comfortable, as positioning the device too far up into the buccal vestibule can cause discomfort and pain. Positioning the drug delivery device on the first and second molars ensures that the device remains planar with only two teeth, which limits its protrusion outward toward the cheek. Locating the drug delivery device in this position also ensures that the delivery tube can wrap around the rearmost molar in patients without a third molar and positions the delivery tube so that it is not bitten. The fastener and drug delivery device can be matched to the dentition of the patient. If the patient has a third molar, the device can be positioned on the opposite side of the mouth that does not have a third molar.

### Pumps

The pumps for the drug delivery devices are suitable for miniature devices carried safely and comfortably in the mouth. Any suitable pump may be used. The pump and the drug reservoir may be distinct. Miniature pumps are advantageous for placement in the mouth. For example, the extruded fluid including the drug may occupy more than 33%, 50%, 66%, or 75% of the total volume of the drug delivery device.

Pumps that do not require a battery can be smaller and have fewer moving parts than battery-requiring electrical pumps. One group of nonelectric disposable pumps is based on the physical principle that mechanical restriction within the flow path by a flow restrictor can determine the flow rate of a pressurized fluid. The restriction of flow may be provided by an orifice (e.g., in the drug reservoir), by narrow-bore tubing (such as a metal, glass or plastic pipe), or by a channel, or by a capillary, or a flow-controlling nozzle. Optionally, the flow-controlling nozzles, channels, or tubes can be made of a plastic such as an engineering plastic, or made of a metal or a ceramic such as a glass. The flow restrictor can have an internal diameter smaller than 1 mm or 2 mm and larger than 0.05 mm and a length between 0.25 cm and 10 cm. In particular embodiments, the flow restrictor can have an internal diameter smaller than 0.7 mm and larger than 0.2 mm. Preferred internal diameters are 0.1 —2 mm (0.1 — 0.7 mm, 0.2 — 0.5 mm, 0.5 - 0.75 mm, 0.75 - 1.0 mm, 1.0 - 1.5 mm, or 1.5 - 2.0 mm) and preferred lengths are 0.25 -5 cm (such as 1 - 2.5 cm, 1 - 5 cm, 0.25 - 0.5 cm, 0.5 - 0.75 cm, 0.75 - 1 cm, 1 - 2 cm, 2 - 3 cm, 3 - 4 cm, or 4 - 5 cm).

Flow rate can be affected by the pressure gradient across the flow restrictor and by fluid viscosity. A significant source of inaccuracy in existing pump products can be that viscosity is strongly affected by temperature. An important benefit of carrying within the mouth the drug delivery devices is that the temperature is held nearly constant at about 37° C., thereby minimizing variations in the rheological properties (such as viscosity and slip rate) and therefore in the infusion rate. The nearly constant about 37° C. is also advantageous in maintaining a stable pumping pressure when a gas, such as from a liquid propellant, is used to drive the pump.

The formulations used in the devices and methods of the invention can be viscous suspensions. Use of viscous suspensions is often desired to achieve the small volumes, high concentrations, uniform drug dispersion, storage stability, and operational stability desired for the drugs and methods of the invention. Consequently, it is often desired to employ pump mechanisms that can provide the pressures required to pump the viscous fluids.

The pressure difference required to extrude the viscous fluid, typically a paste, can be between 2 and 20 bar, such as between 3 and 12 bar, or such as between 4 and 8 bar. The pressurizing gas is typically in equilibrium with liquefied propellant.

### Gas-Driven Infusion Pumps

In one embodiment, a gas-driven drug delivery device includes two or more compartments, with pressurized gas in at least one compartment and the suspension to be administered in at least one separate drug reservoir. The pressurized gas provides the driving force. The two compartments are separated by a movable member (such as a flexible and/or deformable diaphragm) that transmits the force from the gas compartment to the suspension.

The housing containing the two compartments is typically constructed to have a fixed volume that does not vary significantly as the drug is dispensed and the internal pressure declines in the compartment containing the pressurized gas.

In a preferred embodiment, the drug delivery device includes a single component propellant in one compartment and the drug in a second compartment, the propellant boiling at sea level atmospheric pressure at a temperature less than about 37° C. At this temperature, the vapor-pressure of the liquefied propellant is typically greater than 2 bar pressure. Because upon its evaporation the volume of the liquid propellant increases greatly, the volume of the propellant compartment is small relative to the drug comprising fluid volume, typically less than ¼, ⅕^{th}, or ⅛^{th} of the drug comprising fluid (e.g., paste) volume. In this embodiment, a propellant-driven drug delivery device can include a drug reservoir with a pressure-liquefied propellant, i.e., a propellant-containing compartment within the drug delivery device, such that the pressurized, volatile, propellant liquid and the fluid including the infused drug reside in the different compartments.

In a preferred embodiment, the propellant and a solid or fluid drug are contained within a rigid metal-walled housing, for example of titanium or titanium alloy or iron alloy or stainless steel, that does not significantly deform under the pressure of the propellant. The housing includes a drug reservoir. The propellant and the drug are separated within the housing by a deformable diaphragm, transmitting the pressure of the propellant vapor to the drug comprising fluid, typically a deformable semi-rigid paste. The diaphragm may comprise a pinhole free a ductile metal foil, such as a silver foil, or a foil of an austenitic stainless steel, typically of a thickness between 10 µm and 250 µm, e.g., between 20 µm and 125 µm, such as between 25 µm and 75 µm. For hermeticity, the rims of the propellant housing and the metal diaphragm may be welded and residual pinholes in the weld, if any, may be sealed by applying a sealant on the exterior of the weld. The sealant can be polymeric, for example polysiloxane-comprising.

Alternatively, the compartment separating deformable diaphragm can comprise a metalized polymer, such as three-layered polymer-aluminum-polymer. It can be adhered to the rim of the propellant compartment and/or to the rim of the drug comprising fluid compartment using an adhesive, such as an epoxy, polyurethane or polyacrylate adhesive.

Examples of continuously subcutaneously drug infusing compressed air or Freon ™ pressurized pumps include those described in U.S. Pat. Nos. 4,265,241, 4,373,527, 4,781,688, 4,931,050, 4,978,338, 5,061,242, 5,067,943, 5,176,641, 6,740,059, and 7,250,037, each of which is incorporated herein by reference. When the reservoir is refillable and when the pumping is by pressurization, the reservoir can be pressurized upon its refilling.

An example of a propellant-driven, implanted medication infusion pump is the Codman pump described in U.S. Pat. No. 7,905,878, European Patent Nos. EP 2177792 B1 and EP 1527794 B1, each of which is incorporated herein by reference.

To provide different patients with different dose rates, fluids with different drug concentrations can be placed in the reservoirs, thereby not necessitating modifications to the drug delivery device or to the flow rate. Alternatively, the drug concentration in the reservoir can be held constant and the flow rate can be changed, for example by changing the diameter or length of the flow restrictor.

Exemplary volatile propellant compounds for use in the devices include hydrocarbons (e.g., 2-methylpropane; pentane; 1-pentene; trans-2-pentene; trans-dimethylcyclopropane; ethylcyclopropane; 1,4 -pentadiene; 2-methyl-1,3-butadiene; and methyl-1-butane; 2-butyne); halocarbons (e.g., trichlorofluoromethane; difluoromethane; 1,1-dichloro-1-fluoroethane; 2,2-dichloro-1,1,1-trifluoroethane; 1-fluorobutane; 2-fluorobutane; perfluoropentane; 1,1-dichloroethylene; cis-1-chloropropene; and 2-chloropropene); esters (e.g., methyl formate); ethers (e.g., dimethyl ether), and hydrofluoroalkanes. Preferred propellants are those approved by the FDA for use in medication inhalers, such as 1,1,1,2 tetrafluoroethane (R-134a); and 1,1,1,2,3,3,3 heptafluoropropane, sold as R-227ea. Also preferred are propellants approved by the FDA for topical applications, such as 1,1,1,3,3,3 hexafluoropropane (sold as R-236fa); and propellants approved for use in food and over the counter anticarie drug products, such as octafluorocyclobutane or isopentane.

Exemplary pressurized liquid propellants and their vapor pressures at 37° C. are listed in Table 2.

TABLE 2

| Propellant | Approximate Pressure, bars at 37° C. |
|---|---|
| diethyl ether | 1.1 |
| 1-fluorobutane | 1.3 |
| isopentane | 1.4 |
| 2-fluorobutane | 1.6 |
| 1,2-difluoroethane | 1.9 |
| neopentane | 2.4 |
| methyl ethyl ether | 3 |
| 2-butene | 3.2 |
| butane | 3.5 |
| 1-fluoropropane | 4.1 |
| 1-butene | 4.2 |
| 2-fluoropropane | 5 |
| 1,1-difluoroethane | 8.4 |
| propane | 12.8 |
| propene | 15.5 |
| 1,1,1,2-tetrafluoroethane | 9.3 |
| 1,1,1,2,3,3,3-hepta-fluoropropane | 6.4 |
| 1,1,1,3,3,3 hexafluoropropane | 4.0 |
| octafluorocyclobutane | 4.3 |

### Propellant-Driven Pumps

The following sections provide additional details on designs and manufacturing processes of propellant-driven pumps for the delivery of pharmaceutical compositions including LD/CD pastes. It will be recognized that similar designs and manufacturing processes may be used with other pumps and drug formulations.

The devices can be propellant-pumped, rigid walled, intraoral, continuously drug delivering devices having a drug compartment and a propellant compartment separated by an optionally metallic diaphragm. In one embodiment, the device for continuous or semi-continuous intraoral drug administration is configured to be removably inserted in a patient’s mouth. The drug delivery device includes a chamber containing a propellant, a chamber containing a drug-including fluid such as a paste, and a flexible and/or deformable diaphragm separating the propellant chamber from the drug chamber. The housing of the device can be rigid and can be gas and liquid impermeable, for example impermeable to gaseous and liquid propellant, air, water vapor, liquid water, saliva and/or gaseous helium. In a preferred embodiment, the rigid housing forms a wall of a chamber containing the drug-including fluid and a wall of a chamber containing the propellant, and the two chambers are separated by a diaphragm. The separating diaphragm includes a metal, i.e., the diaphragm can be metallic or it can comprise a metallized polymer. The device dispenses at least 50% (e.g., 50% - 99%, 60% - 95%, 75% - 95%, 51% - 60%, 61% - 70%, 71% - 80%, 81% - 90%, 91% - 95%, or 95% - 99%) of the weight of the drug-including fluid (e.g., paste) in the chamber, preferably while the rate of drug delivery, meaning the flow rate or extrusion rate, varies by less than ±20% (e.g., less than ±15%, less than ±10%, or less than ±5%) over a period of greater than or equal to 4, 8, 16, or 24 hours.

The rigid wall of the drug and the propellant including chambers (which can comprise part of the housing) can be strong, dense and it can be metallic. In a preferred embodiment, the rigid housing forms a wall of the drug-containing chamber and/or a wall of the propellant-containing chamber. The rigid housing of the chamber wall can be strong and includes a metal, ceramic, or a composite of a polymer reinforced by fibers. The fibers reinforcing the polymer can include, for example, carbon fibers, glass fibers, or metal fibers. The housing can include a material having at about 25±3° C. a tensile yield strength greater than 100 MPa, such as greater than 200 MPa, 300 MPa, 400 MPa, or 500 MPa; and/or the housing can include a material having at 25±3° C. a modulus of elasticity (Young’s modulus) greater than 30 GPa such as greater than 50 GPa, 75 GPa, or 100 GPa; and/or the housing can include a material having at 25±3° C. a Brinell hardness greater than 200 MPa, such as greater than 400 MPa or 600 MPa; and/or the housing can include a material having at 25±3° C. a density greater than 2.5 g/cm³, such as greater than 3.5 g/cm³, such as about equal to or greater than 4.5 g/cm³, 5.5 g/cm³, 6.5 g/cm³, or 7.5 g/cm³. When metallic, the metal of the housing can be selected from the group titanium, iron, aluminum, molybdenum, or tungsten, or an alloy of titanium, iron, molybdenum, or tungsten; it can be formed of, for example, titanium or an alloy of titanium or a stainless steel, such as an austenitic stainless steel.

The diaphragm separating the chamber containing the drug-including fluid from the chamber containing the propellant can be a deformable metal foil or it can include a flexible and/or deformable metal foil. In a preferred embodiment, the diaphragm separating the chamber containing the drug-including fluid from the chamber containing the propellant can be metallic or it can include a metallic layer. It can be a deformable, pinhole-free, metal foil or it can comprise a metal layer. The density of the diaphragm metal can be greater than 2.0 g per cm³ at 25° C. It can be for example greater than 2.5 g per cm³, such as greater than 4.0 g per cm³, 7.0 g per cm³, or 10.0 g per cm³ at 25° C. Optionally, the tensile strength of the diaphragm material can be greater than 25 MPa, for example it can be greater than 50 MPa, 75 MPa, or 100 MPa at 25±3° C. and/or its elastic modulus can be greater than about 20 GPa, such as greater than 30 GPa, 40 GPa, or 50 GPa. The metallic diaphragm can include, for example, a foil of silver or an alloy of silver; or it can include a layer of aluminum or an alloy of aluminum; or it can include a layer of magnesium or an alloy of magnesium; or it can include a layer of titanium or an alloy of titanium; or it can include a foil of stainless steel. The metal foil (e.g. silver or stainless steel) diaphragm can be pinhole-free; the metal layer (e.g. of titanium, aluminum or magnesium) can have pinholes when in contact with a polymer layer, optionally on both of its sides. In some embodiments, for example when both the housing wall and the diaphragm are of similar metals such as stainless steels, the rims of the diaphragm can be attached to the rims of either or both the propellant and drug housings by welding. When the diaphragm comprises metal and polymer layers, its rim can be adhered to those of the housings using an adhesive, such as a two-part epoxy, a polyacrylate or a polyurethane.

The pump can further include a sealable port for injecting the propellant, e.g., using a needle.

The housing can be made of two or more parts joined together. The parts may be joined together by welding (optionally with a diaphragm) or by using an adhesive.

The interior housing wall of the propellant chamber and interior housing wall of drug chamber can be substantially mirror images of each other, meaning that they can be substantially symmetrical with respect to a central plane, excepting that their ports differ and an interior housing wall of the drug chamber may have grooves or similar flow-enhancing features while the mirroring interior housing wall of the propellant chamber may not have grooves or similar flow-enhancing features.

In a preferred embodiment the housing wall of the drug chamber can include a sealable port that allows for the introduction of a pharmaceutical composition. The port may be temporarily or permanently sealed prior to or after the filling process, e.g., by a plug, grommet, or septum. A port may also be used for delivery of the drug during operation of the device, e.g., by attaching a plugged drug delivery tube.

Optionally, the flow restrictor (e.g., nozzles, channels, or tubes) can be made of a plastic, such as an engineering plastic. The flow restrictor can have an internal diameter smaller than 1 mm or 2 mm and larger than 0.05 mm and a length between 0.25 cm and 10 cm. In particular embodiments, the flow restrictor can have an internal diameter smaller than 0.7 mm and larger than 0.2 mm. Preferred internal diameters are 0.1 - 2 mm (0.1 - 0.7 mm, 0.2 - 0.5 mm, 0.5 - 0.75 mm, 0.75 - 1.0 mm, 1.0 - 1.5 mm, or 1.5 - 2.0 mm) and preferred lengths are 0.25 - 5 cm (such as 1 - 2.5 cm, 1 - 5 cm, 0.25 - 0.5 cm, 0.5 -0.75 cm, 0.75 - 1 cm, 1 - 2 cm, 2 - 3 cm, 3 - 4 cm, or 4 - 5 cm).

The housing wall of the propellant chamber can include a sealable port (e.g., containing a grommet, septum, or similar resealable member) for filling the propellant chamber with propellant. A propellant delivery nozzle can be inserted into the septum and the propellant chamber is filled. Preferably, the drug chamber is filled first and the propellant chamber is subsequently filled.

Patient compliance depends on the drug delivery device and retainer being comfortable when worn in the mouth. Preferably, the system does not substantially affect the appearance of the wearer, impede speech, or impede swallowing and drinking. For comfort and in order to avoid substantial change in the appearance of the face of the wearer the oral pump may have a substantially obround shape. An exemplary location of the pump in the mouth is a maxillary location. In general, it is preferred that the pump and/or its drug outlet be located such that the likelihood of excessive drug accumulation in the buccal vestibule is avoided. In order to avoid irritation of tissue the surfaces of the pump are smooth. For examples, pump surfaces contacting buccal tissue may have protrusions that are less than about 100 µm, e.g., less than about 30 µm, 10 µm, 5 µm, or 1 µm.

The pump may contain between about 0.1 mL and about 2 mL of the drug-including fluid, such as between about 0.2 mL and about 1.2 mL, for example between about 0.6 mL and about 1 mL. An exemplary pump with a 0.8 mL drug reservoir contains about 1 g of an about 1.25 g/mL density composition. In some compositions, there can be 1250 mg/mL of the mostly solid comprising pharmaceutical composition, the solid being mostly the solid drug itself or mostly solid excipient. When the solid is a drug of about 1.5 g/mL density such as LD or CD, the 0.8 mL reservoir can contain about 0.64 g of mostly solid drug.

The pump can be, for example, substantially obround shaped or it can be substantially flattened teardrop shaped. The dimensions of the substantially obround-shaped pump are width, measured from the vestibular surface of the teeth outward, height measured in the direction of tooth eruption, and length measured along the direction of a series of teeth, typically including a molar. The width (outer dimension, OD) of the pump housing can be between about 3 mm and about 10 mm; its height (OD) can be between about 5 mm and about 18 mm; its length (OD) can be between about 10 mm and about 30 mm. Preferably, the length of the pump housing can be such that the pump housing spans one or two teeth, but not three teeth. The thickness of the wall of the rigid housing can be between about 0.2 mm and about 2 mm, such as between about 0.3 mm and about 1.0 mm.

The width of the substantially flattened teardrop shaped pump, its length and the thickness of the housing of the wall can be similar to those of the obround pump. The height of its anterior side when residing in the buccal vestibule can be less than the height of its posterior side. The posterior side can be, for examples, between 1.1 times and twice as high as the anterior side, such as between 1.3 times and 1.8 times as high, e.g., between 1.4 and 1.6 times as high.

In one embodiment the metallic diaphragm is about uniformly thick and it is free of pinholes. The thickness of the pinhole-free metallic diaphragm can be between about 10 µm and about 1 mm. The diaphragm can be, for example, between about 10 µm and 250 µm, e.g., between 20 µm and 125 µm, such as between 25 µm and 75 µm. The thickness and the associated rigidity of the diaphragm, meaning its resistance to change of shape under stress, can vary by less than ±25 % across the diaphragm, such as by less than ±10 %. In some embodiments the rim of the diaphragm is thicker than the about uniformly thick center in order to facilitate sealing. The about uniformly thick center can constitute about 80 % or more of the area of the diaphragm, the thicker rim constituting typically less than about 20% of the area of the diaphragm. The rim of the diaphragm can be more than 1.5 times as thick as its center, e.g., 1.5 — 2 times as thick as the center, or 2 — 3 times as thick, or more than 3 times thicker than the center.

The peripheral rim of the diaphragm is shaped and sized to match the peripheral rim of the central cross sectional plane of the typically obround or flattened teardrop shaped housing. The diaphragm can be made, for example, by forcing a sheet of metal, such as annealed about pure silver foil of a thickness between 0.02 mm and 0.10 mm into a mold. Alternatively, the diaphragm can be made by stamping a formable metal foil or sheet, typically of a thickness between 0.02 mm and 0.10 mm. Parameters that can affect formability include the strain, or work-hardening, exponent of the metal (termed its n-value) and the strain ratio in the width and thickness directions (termed its r-value). Typical r-values of the silver of which the diaphragms are made are from 0.75 to 1.0 and typical n-values are from 0.2 to 0.4. The height of the stamped, metallic, optionally obround, cup-shaped diaphragm (matching about the width of the housing) can be between about 3 mm and about 10 mm; its width (matching the height of the housing) can be between about 5 mm and about 18 mm; and its length can be between about 10 mm and about 30 mm. The optionally obround diaphragm may be planar, folded, pleated, or scored. It can be formed, for example, by hydroforming or by stamping, optionally with heating by hot-stamping, or by deep drawing, optionally with heating.

Optionally, the rims of the flexible and/or deformable metallic diaphragm separating the drug and propellant chambers can be welded to rims of housings to form hermetically sealed propellant chambers and hermetically sealed drug chambers after the filling port of the propellant chamber and the filling and delivery ports of the drug comprising chamber are hermetically closed.

The housing wall of the drug including chamber can include one or more sealable or sealed ports for filling and for drug delivery. The propellant containing chamber can be hermetically sealed and can include a hermetically sealable or sealed port for filling with propellant.

When stored, the pump can be hermetically sealed. When in use, the drug can flow or be extruded through the one, two, or more drug delivery ports.

For hermetic enclosure the drug chamber, propellant chamber, and diaphragm can be joined by a hermetically sealing weld, the hermetically sealing weld preventing, for example, the influx of water, of saliva, of air, or of water vapor, or the out-flux of water vapor, or the out-flux of any constituent of the drug including composition from the drug chamber, as well as the out-flux of propellant from the propellant chamber during the rated shelf-life of the device, which can be longer than 3 months, such as longer than 6, 12, 18, or 24 months. Optionally, the weld can reduce or prevent the influx of helium into and/or out-flux of helium from the drug-including chamber, and/or from the propellant-including chamber, or from both chambers. The weld can be a weld between rims of the metallic housings and/or between rims of the housings and the metallic diaphragm, where the welded metals have about similar thermal expansion coefficients. The weld can be, for example, between rims of iron alloy housings, and/or between an iron alloy housing rim and an iron alloy diaphragm rim, steel the welded iron alloys exemplified by a stainless steel, such as an austenitic stainless steel. The welding method can be, for example, arc welding, resistance welding, laser welding, or electron beam welding. If the resulting weld is porous, it can be made hermetically sealing by applying to it a polymeric sealant, such as a polysiloxane comprising sealant.

An exemplary drug delivery device for constant rate, continuous intraoral delivery of a drug-containing fluid is shown in FIGS. 1 and 2. The components of the device include propellant housing 1 and drug housing 3, both of which can be made of a metal, such as titanium, iron, molybdenum, or tungsten, or an alloy of titanium, iron, molybdenum, or tungsten, or a steel, such as a stainless steel, particularly a ductile stainless steel like 304, 308 or 316L stainless steel. Each of propellant housing 1 and drug housing 3 have a wall thickness between 0.25 mm and 0.5 mm (such as 0.25 mm to 0.3 mm, 0.25 mm to 0.35 mm, 0.25 mm to 0.4 mm, 0.25 mm to 0.45 mm, 0.3 mm to 0.5 mm, 0.35 mm to 0.5 mm, 0.4 mm to 0.5 mm, or 0.45 mm to 0.5 mm). The device also includes diaphragm 2, which may be made of metal or contain a metal layer. For example, metallic diaphragm 2 can be a foil of annealed silver, or of an annealed austenitic stainless steel, such as 304, 308 or 316L stainless steel Metallic foil diaphragm 2 can be 0.025 mm to 0.25 mm thick (such as 0.025 mm to 0.05 mm, 0.05 mm to 0.075 mm, 0.025 mm to 0.035 mm, 0.025 mm to 0.05 mm, 0.025 mm to 0.75 mm, 0.025 mm to 0.1 mm, 0.025 mm to 0.125 mm, 0.025 mm to 0.15, 0.025 mm to 0.175 mm, 0.025 mm to 0.2 mm, 0.025 mm to 0.225 mm).

Septum eyelet 4 is inserted into propellant housing 1 and can be made of metal, typically titanium or titanium alloy. The thickness of septum eyelet 4 is typically between 0.25 mm and 0.5 mm (such as 0.25 mm to 0.3 mm, 0.25 mm to 0.35 mm, 0.25 mm to 0.4 mm, 0.25 mm to 0.45 mm, 0.3 mm to 0.5 mm, 0.35 mm to 0.5 mm, 0.4 mm to 0.5 mm, or 0.45 mm to 0.5 mm). Septum 5 can be inserted into septum eyelet 4, and may optionally contain a rubber, such as nitrile rubber. Elastomeric plug 6 can be inserted into drug housing 3 and can be an elastomeric fluoropolymer plug, exemplified by a Viton™ plug. The drug delivery device may contain one or more (e.g., one, two, three, or more) flow restrictors. FIG. 2 shows an embodiment of the device containing two flow restrictors 7 of different lengths, in which one is substantially shorter than the other (e.g., one flow restrictor extends further into the drug chamber than the other). The flow restrictors regulate flux (e.g., control the administration rate) of the drug-containing fluid, and are mounted in coupling adaptor 8, which is connected to delivery tube 9. In a device containing two or more flow restrictors, the flow restrictors may be of the same length or of different lengths and may have the same internal diameter or different internal diameters. The flow restrictor may be made of metal, glass, or plastic and, in embodiments in which the device contains two or more flow restrictors, the flow restrictors may be made of the same or of different material. Preferably, in embodiments in which the drug delivery device contains two or more flow restrictors, all of the flow restrictors are made of the same material. The flow restrictor may be made of any polymer that has a low water permeability and that is chemically inert. In some embodiments, the flow restrictor is made of a thermoplastic polymer, such as polypropylene, polyethylene, polyvinyl chloride, polystyrene, acrylic, polyamide (nylon), acrylonitrile butadiene styrene, polycarbonate, or polyvinylidene fluoride. In some embodiments, the flow restrictor is made of a thermoset polymer. In some embodiments, the flow restrictor is made of a thermoplastic polyester, such as polybutylene terephthalate (PBT), polyethylene terephthalate (PET),or polyethylene furanoate (PEF). In some embodiments, the flow restrictor is made of PET. FIG. 1 shows distal, long flow restrictor 7a and its coupling adaptor 8. In embodiments in which the drug delivery device contains two or more flow restrictors, the flux (e.g., flow rate or administration rate) through the two or more flow restrictors may be about the same or it may be different. Preferably, in embodiments in which the drug delivery device contains two or more flow restrictors, the flux (e.g., flow rate) through each flow restrictor will be about the same. When two or more flow restrictors of different lengths (e.g., two flow restrictors of different lengths that extend into different regions of the drug chamber) are included in the device, each flow restrictor can deliver drug-containing fluid from a different part of the drug chamber. When the device contains two or more flow restrictors (e.g., two flow restrictors), the lengths of the flow restrictors are such that each flow restrictor targets (e.g., terminates at) a different location in the drug chamber on one end and does not extend into the bend of the delivery tube on the other end. The internal diameters of the two or more flow restrictors can be selected to achieve a desired flow rate, and the internal diameter of each flow restrictor can be sized such that the flow rate between the two or more flow restrictors is matched as closely as possible (e.g., the flow rate through each flow restrictor is approximately the same) to prevent different regions of the drug chamber from emptying at different rates (e.g., to ensure that the regions of the drug chamber in which the two or more flow restrictors terminate empty at approximately the same rate). A pump may contain two flow restrictors fluidically connected to the drug reservoir, in which the first flow restrictor has a length of 6 - 12 mm (e.g., 6 - 11 mm, 6 -10 mm, 6 - 9 mm, 6 - 8 mm, 6 - 7 mm, 7 -11 mm, 7 - 10 mm, 7 - 9 mm, or 7 - 8 mm) and an internal diameter of 0.0100 - 0.0120 inches (e.g., 0.0115 inches), and the second flow restrictor has a length of 14 - 24 mm (e.g., 16 - 22 mm, 16 - 20 mm, 16 -18 mm, 16 - 17 mm, 17 - 22 mm, 17 -20 mm, 17 - 22 mm, 18 - 20 mm, or 18 - 22 mm) and an internal diameter of 0.0130 - 0.0145 inches (e.g., 0.0130 or 0.0135 inches). Exemplary combinations of flow restrictor dimensions for pairs of flow restrictors that can be used in the devices described herein are provided in Table 3 below, where ID means internal diameter.

TABLE 3

Exemplary polymeric flow restrictor dimensions

| Exemplary configurations | Proximal restrictor ID (in) | Proximal restrictor length (mm) | Distal restrictor ID (in) | Distal restrictor length (mm) |
|---|---|---|---|---|
| Configuration 1 | 0.0115 | 11 | 0.013 | 22 |
| Configuration 2 | 0.0115 | 11 | 0.0135 | 20 |
| Configuration 3 | 0.0115 | 10 | 0.0135 | 18 |
| Configuration 4 | 0.0115 | 9 | 0.0135 | 17 |
| Configuration 5 | 0.0115 | 8 | 0.0135 | 16 |
| Configuration 6 | 0.0115 | 7 | 0.0135 | 16 |

Delivery tube 9 and 10, also termed outlet tube, can have a length of 0.5 cm to 5 cm (such as 0.5 cm to 1.0 cm, 0.5 cm to 1.5 cm, 0.5 cm to 2.0 cm, 0.5 cm to 2.5 cm, 0.5 cm to 3.0 cm, 0.5 cm to 3.5 cm, 0.5 cm to 4.0 cm, 0.5 cm to 4.5 cm, 1.0 cm to 5.0 cm, 1.5 cm to 5.0 cm, 2.0 cm to 5.0 cm, 2.5 cm to 5.0 cm, 3.0 cm to 5.0 cm, 3.5 cm to 5.0 cm, 4.0 cm to 5.0 cm, or 4.5 cm to 5.0 cm) and an internal diameter of 0.010 inches to 0.08 inches (e.g., 0.010 inches to 0.075 inches, 0.010 inches to 0.070 inches, 0.010 inches to 0.065 inches, 0.010 inches to 0.060 inches, 0.010 inches to 0.055 inches, 0.010 inches to 0.050 inches, 0.010 inches to 0.045 inches, 0.010 inches to 0.040 inches, 0.010 inches to 0.035 inches, 0.010 inches to 0.030 inches, 0.010 inches to 0.025 inches, 0.010 inches to 0.020 inches, 0.015 inches to 0.080 inches, 0.020 inches to 0.080 inches, 0.025 inches to 0.080 inches, 0.030 inches to 0.080 inches, 0.035 inches to 0.080 inches, 0.040 inches to 0.080 inches, 0.045 inches to 0.080 inches, 0.050 inches to 0.080 inches, 0.055 inches to 0.080 inches, 0.060 inches to 0.080 inches, 0.065 inches to 0.080 inches, or 0.070 inches to 0.080 inches). The delivery tube can be multilayered. For example, the delivery tube can be made of a laminated PET/polyurethane tubing. A typical delivery tube is shown in FIGS. 1 and 2. Outlet tube 9 and 10 can comprise an outer biocompatible polymer (e.g., compatible with living tissue, for example, the material does not cause sensitivity or irritation of the oral mucosa and does not cause systemic toxicity or cytotoxicity) and an inner polymer that can prevent substantial loss of water from, meaning drying out of, the fluid pharmaceutical composition in the outlet tube. The tube can be reversibly compressible, able to revert to about its original shape after a patient bites on it or after it is pinched or kinked. The outlet tube can be flexible, comfortable to wear without impeding speech, and can re-acquire its inner diameter after being kinked or pressed on to stop extrusion of the fluid pharmaceutical composition during storage. If the delivery tube is bitten, pinched, kinked, or otherwise compressed, the rate of drug delivery can change by less than 10% (e.g., less than 10%, 9%, 8%, 7%, 6%, 5%, 4%, 3%, 2%, or 1%) after the biting, pinching, kinking, or compression ends (e.g., the rate of administration once the delivery tube recovers from the compression, e.g., as compared to the rate of administration before the compression). It was found that a multilayered delivery tube was better able to recover from being kinked, particularly a multilayered delivery tube containing an external elastomeric layer.

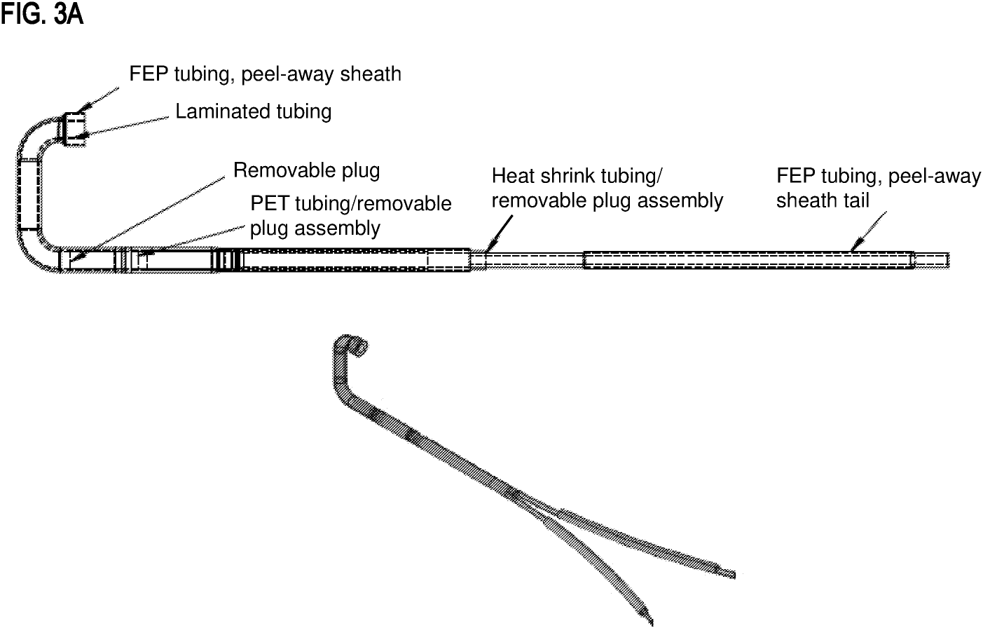

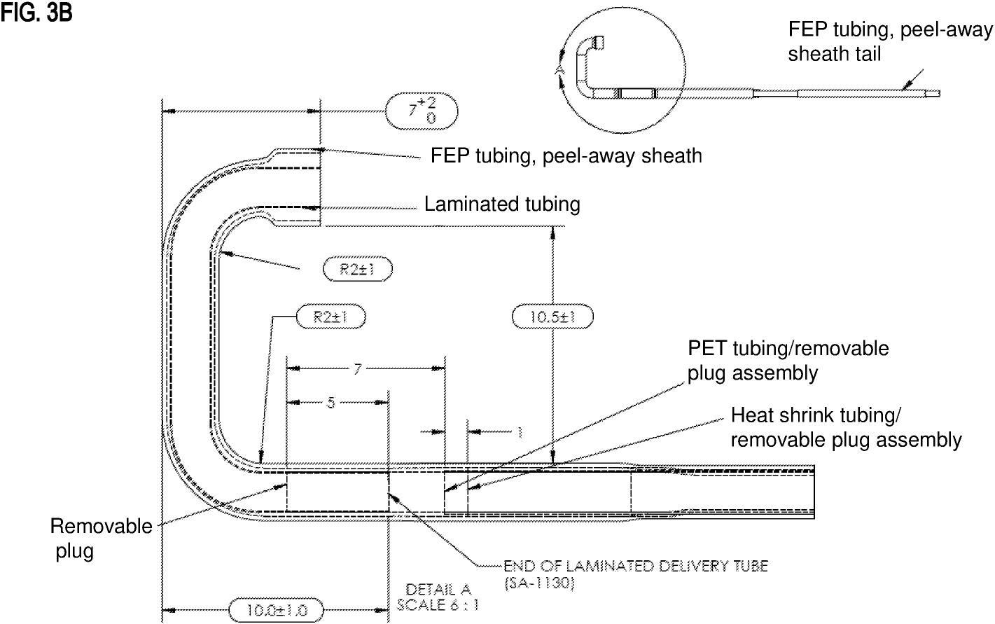

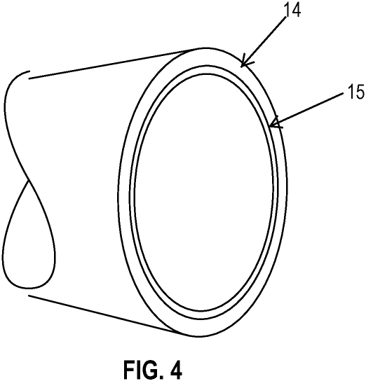

The delivery tube can contain two laminated polymer layers: an outer layer, typically made of an elastomeric biocompatible polymer, such as a polyurethane or a crosslinked polyethylene glycol (PEG) derivative, such as poly(ethylene glycol) diacrylate or poly(lactide-co-glycolide) (PLGA), and an inner, mechanically strong polymer layer that reduces the permeation of water and is compatible with the components of the drug-containing fluid (e.g., its water, oil, and surfactant), such as polyethylene terephthalate (PET), high density polyethylene (HDPE), polyvinylidene chloride (PVDC), polytetrafluoroethylene (PTFE), or polymeric fluorinated ethylene propylene (FEP). In some embodiments, the polyurethane is a thermoplastic polyurethane elastomer, such as Pellethane®. An exemplary delivery tube is shown in FIGS. 1-4. FIG. 4 is a cross sectional view of the delivery tube, showing a view of the laminated polymer layers: outer polymer layer 14, which can be a polyurethane layer, chosen for its biocompatibility and elasticity; and inner polymer layer 15, which can be a PET layer, providing radial strength and reducing permeation of water. Inner polymer layer 15 is also compatible with the components of the drug containing-fluid, such as oil, water, and surfactant. The thickness of outer layer 14 can be between 0.05 mm and 0.2 mm (such as 0.05 mm to 0.075 mm, 0.05 mm to 0.1 mm, 0.05 mm to 0.125 mm, 0.05 mm to 0.15 mm, 0.05 mm to 0.175 mm, 0.075 mm to 0.2 mm, 0.1 mm to 0.2 mm, 0.125 mm to 0.2 mm, 0.15 mm to 0.2 mm, or 0.175 mm to 0.2 mm) and the thickness of inner layer 16 can be between 0.025 mm and 0.075 mm (e.g., 0.025 mm to 0.05 mm or 0.05 mm to 0.075 mm). In some embodiments, the thickness of the outer layer of the delivery tube (e.g., the polyurethane layer) is 0.005 inches.

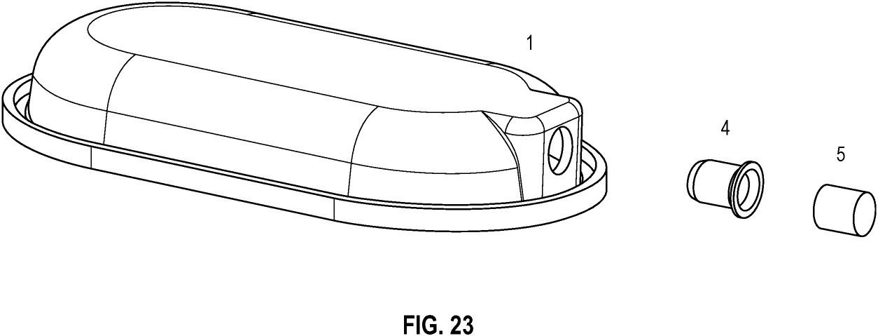

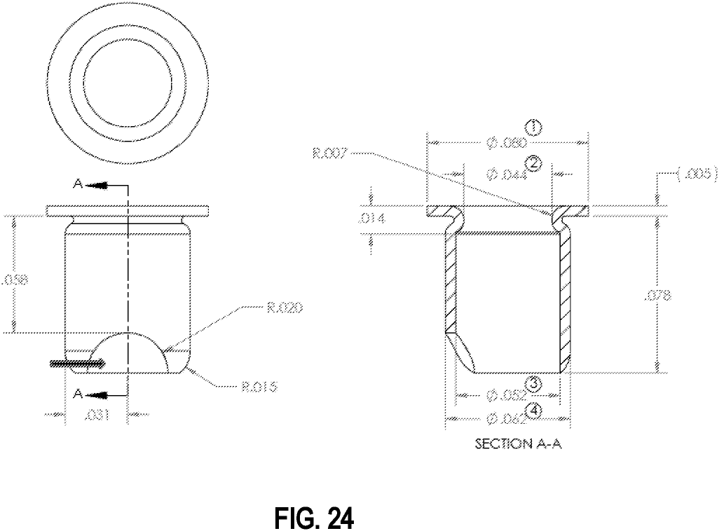

The drug delivery device can contain septa for loading the device with a propellant and a drug-containing fluid (e.g., a suspension, emulsion, or paste) - at least one septum for loading the propellant into a propellant chamber and at least a second septum for filling the drug reservoir with the drug-containing fluid. Both of these septa pass extractables and leachables testing to FDA and EU standards in addition to biocompatibility testing. In the exemplary drug delivery device shown in FIGS. 1 and 2, septum 5 can be used for filling the propellant chamber (the chamber between diaphragm 2 and propellant housing 1) with propellant, and elastomeric plug 6 can be used for sealing the drug chamber (the drug reservoir, which corresponds to the chamber between diaphragm 2 and drug housing 3) after it has been filled with a drug-containing fluid. Components of the septum for filling the propellant chamber are shown in FIG. 23. These include eyelet 4, which is bonded to propellant housing 1, and septum 5, which can be elastomeric and compressible. The diameter of septum 5 can be reduced upon compression, for example, from 0.063 inches to 0.052 inches, allowing its press-fitting into eyelet 4 to form a hermetic seal. The elastomeric material used to make septum 5 can be both biocompatible (e.g., compatible with living tissue, for example, the material does not cause sensitivity or irritation of the oral mucosa and does not cause systemic toxicity or cytotoxicity) and compatible with the liquid propellant (e.g., does not react with the propellant). It can be, for example, a nitrile rubber. The propellant chamber can be filled with propellant by piercing septum 5 with a needle for injection of a propellant, such as 1,1,1,2-tetrafluoroethane. Once the needle is withdrawn, the elastomeric plug (septum 5) seals the propellant compartment hermetically. For example, after injection of about 100 mg 1,1,1,2-tetrafluoroethane, its leakage at about 23 ± 3° C., at which the vapor pressure of 1,1,1,2-tetrafluoroethane is about 5 bars, may not cause a weight loss exceeding 0.2 mg in 24 hours. The plug (septum 5) can be, for example, about 0.085 inches long, can extend beyond the slightly shorter eyelet, and it can flare out (e.g., the septum may be flared), preventing its ejection under the pressure in the propellant chamber. A cut out in eyelet 4, marked with a solid arrow in FIG. 24, can guide the propellant-injecting needle, which can be inserted at an angle so as to prevent piercing of diaphragm 2. An internal rib in the eyelet can increase resistance to injection.

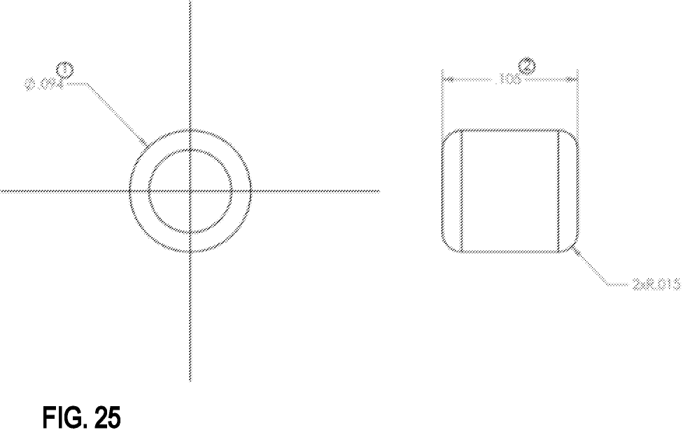

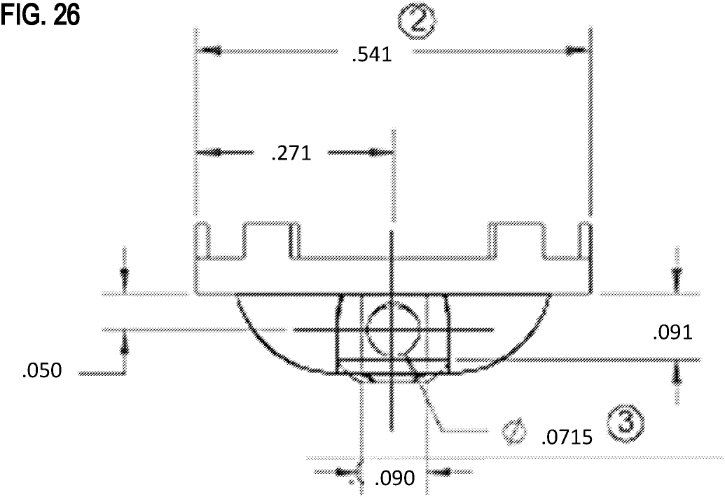

Elastomeric plug 6 can be used to seal the drug chamber after it is filled with the drug-containing fluid. An exemplary drawing of an elastomeric plug for sealing the drug chamber is shown in FIG. 25. The diameter of the plug can be about 0.094 inches and its length can be about 0.105 inches. Drug housing 3 of FIGS. 1 and 2 can include an exemplary port for filling with the drug-containing fluid, as shown in the drawing of FIG. 26, which can be sealed with the exemplary elastomeric plug of FIG. 25. The elastomeric plug can be an injection molded elastomer. The elastomer can be both biocompatible (e.g., compatible with living tissue, for example, the material does not cause sensitivity or irritation of the oral mucosa and does not cause systemic toxicity or cytotoxicity) and compatible with all components of the drug-containing fluid, such as oil, surfactant, and water (e.g., does not react with any of the components of the drug-containing fluid). The elastomeric plug can be made, for example, of a fluorocarbon elastomer, such as Viton™. The exemplary 0.094 inch diameter plug can be compressed to about 0.0715 inches to allow its pressing into the port after the chamber is filled with the drug-containing fluid (e.g., a suspension, emulsion, or paste). In combination with a chamber wall thickness of about 0.015 inches, the plugged port can sustain a pressure of about 12 bar. Although it is exemplified in a device containing a propellant-driven pump, an elastomeric plug can be used for sealing the drug reservoir of any of the drug delivery devices described herein after the drug reservoir is filled with drug-containing fluid.

In a preferred embodiment, greater than 60% (e.g., 75% - 85%, 86% - 95%, or greater than 95%) of the drug-including fluid can be dispensed while the delivery rate varies by less than ±20% (e.g., less than ±15%, ±10%, or ±5%) over a period of greater than or equal to 4 hours (e.g., greater than or equal to 8, 16, or 24 hours).

Typically, neither the metal of the rigid housing nor of the diaphragm may corrode visibly after 3 months when the housing metal and the diaphragm metal are electrically shorted and are immersed in a substantially de-oxygenated 0.1 M citrate buffer solution of about pH 4 at about 23±3° C. The de-oxygenated solution can be a solution kept under nitrogen. Typically, neither the metal of the rigid housing nor of the diaphragm may corrode visibly after 3 months while the housing metal and the diaphragm metal are electrically shorted and are immersed in an air-exposed 0.1 M citrate buffer solution of about pH 4.0 at about 23±3° C. The density of the current flowing between two electrically shorted electrodes of about equal area, one of the metal of the rigid housing, the other of the metal of the diaphragm, can less than 2 µA cm⁻² such as less than 0.5 µA cm⁻², for example less than 0.1 µA cm⁻² when the electrodes are immersed in a substantially de-oxygenated about pH 4 0.1 M citrate buffer solution at 23±3° C. for 24 hours or more.

In order to obtain the desired rate of delivery of the pharmaceutical composition without clogging the flow restrictor (e.g., the nozzle) the apparent viscosity and the particle size of the pharmaceutical composition, the vapor pressure, as well as the diameter and length of the flow restrictor are simultaneously controlled. Table 4 provides exemplary ranges for these simultaneously controlled parameters for an intra-oral drug delivery device and formulation of the invention.

TABLE 4

Exemplary parameter ranges for continuous intra-oral drug delivery devices and formulations

| | Flow Restrictor ID, mm | Flow Restrictor Length, cm | Propellant Vapor Pressure, bar at about 37° C. | Oral Extrusion Rate, mL/hour | Drug or Excipient Particle Size, D₉₀, µm* | Drug or Excipient Particle Size, D₅₀, µm** |
|---|---|---|---|---|---|---|
| Possible | 0.05-3.00 | 0.25-20 | 1.2-50 | 0.001-1.000 | 0.1-200 | 0.1-50 |
| Typical | 0.1-0.7 | 1.0-5.0 | 2.5-15.0 | 0.03-0.5 | 1.0-50 | 0.5-30 |
| Preferred | 0.2-0.5 | 1.00-2.5 | 4.0-10.0 | 0.05-0.2 | 3.0-30 | 2.00-20.0 |

*Measured by light scattering when the particles are suspended in a non-solvent, e.g. with a Malvern Ltd (UK) Mastersizer.
**Typically, the compositions contain solid drug particles and/or excipient particles and can be semirigid deformable pastes

The oral device can continuously or semi-continuously extrude a semisolid drug-comprising composition into the mouth, the composition deforming under pressure; it can also comprise a mechanical pump comprising, for example, a spring, pressurized gas, or propellant. The device can comprise a flow restrictor such as a nozzle, a channel, a tube or any other flow or extrusion restricting component. The rate of extrusion through the nozzle can depend on its internal diameter, on its length, and on the vapor pressure of the liquefied propellant.

The oral device can comprise a semisolid deformable drug-comprising paste, extruded into the mouth at a rate that can be between 0.001 mL/hour and 1.25 mL/hour (e.g., 0.015 - 1.25 mL/hour). The viscosity of the paste, the solution or the suspension can be greater than 100 poise and less than 50,000 poise at about 37° C. The device can comprise a propellant having a vapor pressure at about 37° C. greater than 2 bar and less than 50 bar (e.g., 3 — 10 bar). When a paste comprising drug particles and/or excipient particles is extruded into the mouth, the particle size distribution, measured by light scattering (e.g. with a Malvern Mastersizer after dispersing the paste in a liquid non-solvent) can have a D₉₀ less than 200 µm and a D₅₀ between about 0.5 µm and about 30 µm.

A typical device can comprise a viscous drug-comprising paste, or a viscous orally infused drug-comprising solution, or a viscous orally-infused drug-comprising suspension, extruded or infused into the mouth at a rate that can be between 0.03 mL/hour and 0.5 mL/hour. The typical viscosity of the paste, solution or suspension can be greater than 200 poise and less than 100,000 poise at about 37° C.; its extrusion rate or flow rate can be controlled mostly by a flow restrictor (e.g., nozzle) which can have an internal diameter between 0.1 mm and 0.7 mm and can be between 1 cm and 5 cm long; the typical device can also comprise a mechanical pump. A semirigid deformable drug-comprising paste can be administered at an extrusion rate between 0.001 mL/hour and 1.25 mL/hour; the paste, can have at low shear rate a viscosity greater than 2,000 poise and less than 50,000 poise; the extrusion rate or the flow rate can be controlled mostly by a flow restrictor or pair of flow restrictors; the extrusion or infusion can be driven by a mechanical pump. The mechanical pump can comprise a propellant, the propellant can have a vapor pressure at about 37° C. greater than 2 bar and less than 50 bar. The paste or the suspension or the solution can comprise solid drug and/or excipient particles whose particle size distribution (when dispersed in a non-solvent and when measured by light scattering) can have a D₉₀ less than 200 µm and a D₅₀ between 0.1 µm and 50 µm.

Any of the drug delivery devices described herein can include a drug reservoir described in U.S. Publication Nos. US2016-0278899A1 and US2017-0172961A1, which are incorporated herein by reference in their entirety.

### Delivery Tubes

The drug delivery devices of the invention release the drug-containing fluid contained within the device (e.g., within the drug reservoir) into the mouth via a delivery tube, which is fluidically connected to the drug reservoir. The delivery tube can have a length of 0.5 cm to 5 cm (such as 0.5 cm to 1.0 cm, 0.5 cm to 1.5 cm, 0.5 cm to 2.0 cm, 0.5 cm to 2.5 cm, 0.5 cm to 3.0 cm, 0.5 cm to 3.5 cm, 0.5 cm to 4.0 cm, 0.5 cm to 4.5 cm, 1.0 cm to 5.0 cm, 1.5 cm to 5.0 cm, 2.0 cm to 5.0 cm, 2.5 cm to 5.0 cm, 3.0 cm to 5.0 cm, 3.5 cm to 5.0 cm, 4.0 cm to 5.0 cm, or 4.5 cm to 5.0 cm) and an internal diameter of 0.010 inches to 0.08 inches (e.g., 0.010 inches to 0.075 inches, 0.010 inches to 0.070 inches, 0.010 inches to 0.065 inches, 0.010 inches to 0.060 inches, 0.010 inches to 0.055 inches, 0.010 inches to 0.050 inches, 0.010 inches to 0.045 inches, 0.010 inches to 0.040 inches, 0.010 inches to 0.035 inches, 0.010 inches to 0.030 inches, 0.010 inches to 0.025 inches, 0.010 inches to 0.020 inches, 0.015 inches to 0.080 inches, 0.020 inches to 0.080 inches, 0.025 inches to 0.080 inches, 0.030 inches to 0.080 inches, 0.035 inches to 0.080 inches, 0.040 inches to 0.080 inches, 0.045 inches to 0.080 inches, 0.050 inches to 0.080 inches, 0.055 inches to 0.080 inches, 0.060 inches to 0.080 inches, 0.065 inches to 0.080 inches, or 0.070 inches to 0.080 inches). The delivery tube can be multilayered. For example, the delivery tube can be made of a laminated PET/polyurethane tubing. A typical delivery tube is shown in FIGS. 1 and 2. The tube is shaped and located in the mouth so as not to interfere with speech. When the drug delivery device is located in the cheek pocket and contains a fluid pharmaceutical composition (e.g., a suspension, emulsion, or paste) containing a drug in the drug chamber, also referred to herein as the drug reservoir, the drug-containing fluid is extruded through the outlet tube into the salivated oral cavity on the lingual side of the teeth, avoiding accumulation of the drug-containing fluid in the cheek pocket or in a nearby buccal region. The delivery tube is designed such that the components of the drug-containing fluid are neither absorbed by the tube nor diffuse through the wall of the tube during storage (e.g., during the shelf life before use). The wall of the delivery tube can prevent substantial loss of water from, and drying out of, the fluid pharmaceutical composition when it is in the outlet tube. The tube can be reversibly compressible, able to revert to about its original shape after a patient bites on it or after it is pinched or kinked (e.g., pinched or kinked by a patient during insertion or use). The tube can also be compressed when stored in a storage case, as described below. The outlet tube can be flexible, comfortable to wear without impeding speech, and can re-acquire its inner diameter or a sufficiently large inner diameter after being kinked or pressed on to stop extrusion of the fluid pharmaceutical composition during storage. If the delivery tube is bitten, pinched, kinked, or otherwise compressed, the rate of drug delivery can change by less than 10% (e.g., less than 10%, 9%, 8%, 7%, 6%, 5%, 4%, 3%, 2%, or 1%) after the biting, pinching, kinking, or compression ends (e.g., the flow rate once the delivery tube recovers from the compression is not significantly reduced as compared to the flow rate before the compression). It was found that a multilayered delivery tube was better able to recover from being kinked, particularly a multilayered delivery tube containing an external elastomeric layer.

The delivery tube can contain two laminated polymer layers: an outer layer, typically made of an elastomeric biocompatible polymer, such as a polyurethane or a crosslinked polyethylene glycol (PEG) derivative, such as poly(ethylene glycol) diacrylate or poly(lactide-co-glycolide) (PLGA), and an inner, mechanically strong polymer layer that reduces the permeation of water and is compatible with the components of the drug-containing fluid (e.g., its water, oil, and surfactant), such as polyethylene terephthalate (PET), high density polyethylene (HDPE), polyvinylidene chloride (PVDC), polytetrafluoroethylene (PTFE), or polymeric fluorinated ethylene propylene (FEP). In some embodiments, the polyurethane is a thermoplastic polyurethane elastomer, such as Pellethane®. An exemplary delivery tube is shown in FIGS. 1-4. FIG. 4 is a cross sectional view of the delivery tube, showing a view of the laminated polymer layers: outer polymer layer 14 and inner polymer layer 15. The outer polymer layer is chosen for its biocompatibility and elasticity, and the inner polymer layer is intended to provide radial strength and reduce permeation of water. The inner polymer layer can also be compatible with the components of the drug containing-fluid, such as oil, water, and surfactant. In some embodiments, the outer polymer layer is a polyurethane layer and the inner polymer layer is PET. The thickness of the outer layer can be between 0.05 mm and 0.2 mm (such as 0.05 mm to 0.075 mm, 0.05 mm to 0.1 mm, 0.05 mm to 0.125 mm, 0.05 mm to 0.15 mm, 0.05 mm to 0.175 mm, 0.075 mm to 0.2 mm, 0.1 mm to 0.2 mm, 0.125 mm to 0.2 mm, 0.15 mm to 0.2 mm, or 0.175 mm to 0.2 mm) and the thickness of the inner layer can be between 0.025 mm and 0.075 mm (e.g., 0.025 mm to 0.05 mm or 0.05 mm to 0.075 mm). In some embodiments, the thickness of the outer layer of the delivery tube (e.g., the polyurethane layer) is 0.005 inches. A multilayered, reversibly compressible delivery tube described herein can be included in any drug delivery device, regardless of the type of pump included in the device, including the drug delivery devices described in U.S. Publication Nos. US2016-0278899A1 and US2017-0172961A1, which are incorporated herein in their entirety by reference.

### Strengthening the Device

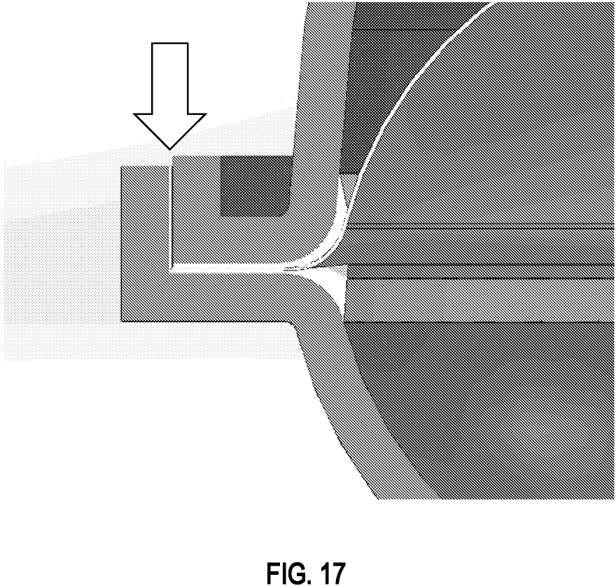

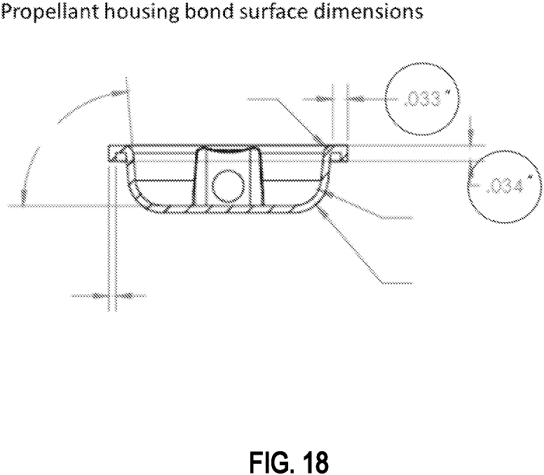

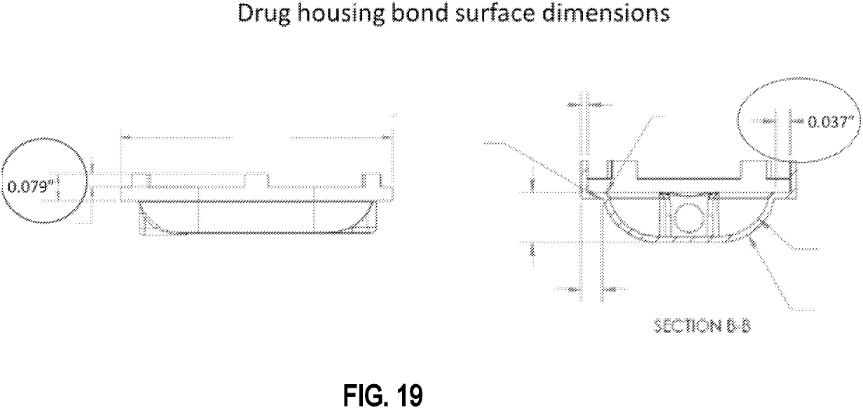

When welding is difficult, for example because of severely mismatched thermal expansion coefficients of the welded metallic parts, or because of embrittlement that can result of conditions of annealing of the weld, or because of excessive corrosion of the weld, resulting of lattice defects and phase changes, the components can be joined using an adhesive. The components of the drug delivery device (e.g., the components that make up the housing of the drug reservoir and/or pump, optionally including a diaphragm) can be joined together using an adhesive. In embodiments in which an adhesive is used to join the components of the device, the components of the device can include rims (e.g., flanges) with a feature, such as a step, that increases the adhesive-bonded area, thereby strengthening the adhesive bonds of the device. This approach can increase the adhered surface area without increasing the size of the device or the amount of space it requires in the mouth. For example, in an embodiment of the drug delivery device that includes a propellant-driven pump having a propellant chamber and a drug chamber, as shown in FIGS. 1 and 2, propellant housing 1 and drug housing 3 can each have rims with a feature, such as a step, as shown in FIG. 17, to increase the adhesive-bonded area. The increase in adhesive-bonded area can prevent failure of the adhesive bond subjected to the force associated with the pressure in the propellant chamber and transmitted, by diaphragm 2 of FIGS. 1 and 2, to the drug chamber containing the drug-containing fluid. When the device is in the mouth at body temperature, the absolute pressure can be between 8 bars and 12 bars, corresponding to a pressure difference at sea level between 7 bars and 11 bars. Different adhesives, such as acrylate, gum, epoxy-type, or a sealant can be used to strengthen the device. Acrylate-type adhesives can include acrylic acid polymer and its ethyl or methyl ester and their copolymers, methacrylic acid and its ethyl or methyl esters and their copolymers, or polymeric 2-ethylhexyl acrylate and its copolymers. The epoxy adhesive can contain two components, one including a compound with two or more epoxide functions, and the other including a compound with two or more amine functions. In some embodiments, the adhesive that is used is a two component adhesive epoxy. The cured epoxy adhesive can be compatible with both the propellant and with components of the drug-containing fluid, such as water, oil, and surfactant. The adhesive (e.g., the epoxy adhesive) can also be biocompatible (e.g., compatible with living tissue, for example, the adhesive does not cause sensitivity or irritation of the oral mucosa and does not cause systemic toxicity or cytotoxicity). Adhered mating parts propellant housing 1 and drug housing 3 of FIGS. 1 and 2, which may be metallic, can sandwich diaphragm 2. Mating flange features in both housings can provide a gap, shown by a hollow arrow in FIG. 17, that can be filled with adhesive. Filling the gap with adhesive can hermetically seal the device. In a propellant-driven pump, such as the pump shown in FIGS. 1 and 2, hermetically sealing the drug delivery device can prevent the propellant and drug-containing fluid from leaking out of their respective chambers under high pressure. Three seams can be sealed by the adhesive: the seam between propellant housing 1 and drug housing 3; the seam between propellant housing 1 and diaphragm 2; and the seam between drug housing 3 and diaphragm 2. FIG. 18 shows the typical dimensions of the flanges (rims) and their steps for strengthening the adhesive bond in a propellant housing, such as propellant housing 1 of FIGS. 1 and 2. FIG. 19 shows the typical dimensions of the flanges (rims) and their steps for strengthening the adhesive bond in a drug housing, such as drug housing 3 of FIGS. 1 and 2.

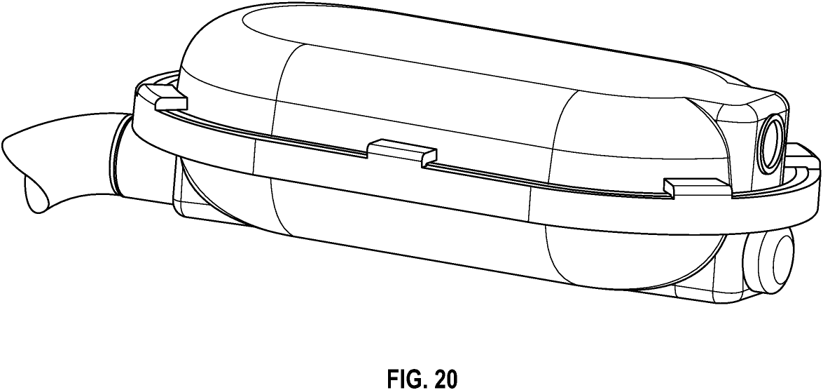

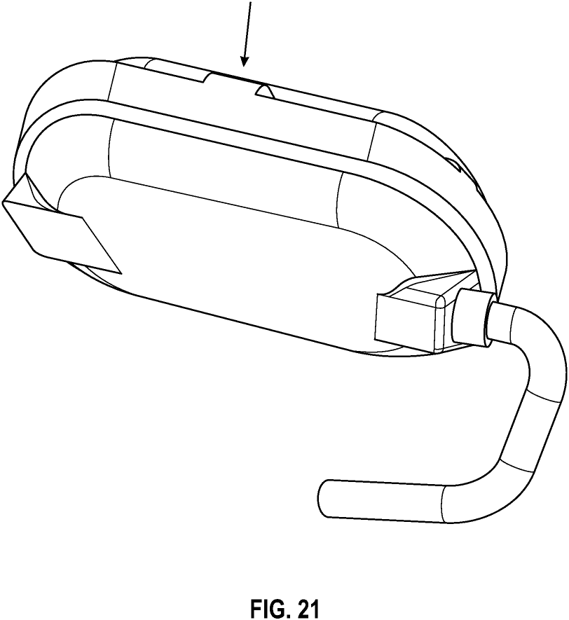

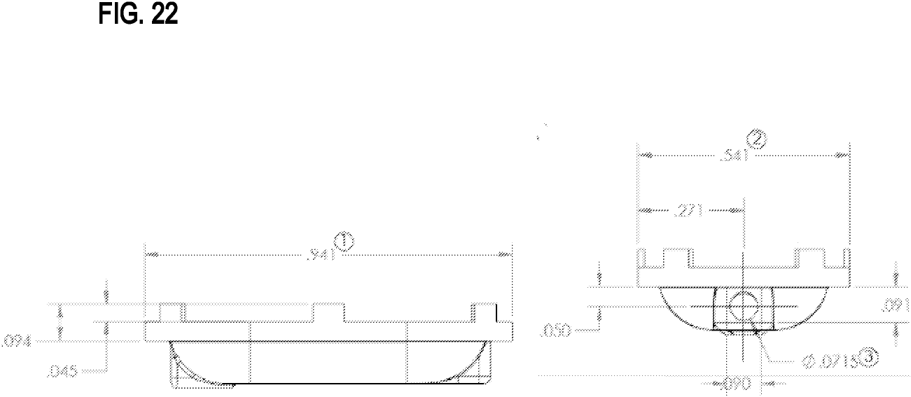

The drug delivery device can also be strengthened by incorporating crimpable tabs into a housing of the device (e.g., into a metallic housing). Crimping the tabs during assembly can provide strength, safety, and assure integrity during and after adhesive curing. Crimping can also increase adhesive bond strength and serve as an extra safety feature, holding the pump together in the event of an adhesive bond failure. Crimpable tabs can be included in a drug delivery device in which the components are joined using adhesive, as described above, or in a drug delivery device in which the components are joined using other means. In a drug delivery device that includes two housings (e.g., a housing for a drug chamber and a housing for a propellant chamber), crimpable tabs are included on one of the two housings. As an example, drug housing 3 of FIGS. 1 and 2 can contain tabs that can be crimped during assembly. FIGS. 20 and 21 each show an assembled drug delivery device in which the tabs have been crimped. FIG. 22 is a set of engineering drawings showing exemplary dimensions of the crimpable tabs.

### Preventing Extrusion of the Drug-Containing Fluid Before Use of the Drug Delivery Device

The drug delivery device can be stored after production and before use. During this time, a removable plug can be positioned within or at the end of the delivery tube to hermetically seal the delivery tube and prevent extrusion of the drug-containing fluid (e.g., suspension, emulsion, or paste) before use. If the drug delivery tube were not hermetically sealed, the drug-containing fluid could leak out of the delivery tube and climb along the delivery tube-removable plug interface. This could lead to a loss of water, which would change the flow rate once the drug delivery device was in use, or drying of the drug-containing fluid in the delivery tube, which could lead to clogs. Using a removable plug that hermetically seals the delivery tube minimizes these potential problems. The removable plug (e.g., an elongated composite plug) can prevent entry of the drug-containing fluid (e.g., paste) into the plugged part of the delivery tube, reducing the area of the drug-containing fluid-contacting surface area through which water permeates, and slowing the eventual drying out of the proximal drug-containing fluid, which is beneficial since drying out of the drug-containing fluid reduces the rate of fluid (e.g., paste) extrusion (e.g., when the drug delivery device is in use). Sheathing of part of the delivery tube, including its plugged zone, further reduces the outflux of water vapor. The elongated plug can additionally be designed to facilitate its removal by the patient or caregiver prior to the use of the device. The removable plug may be hard to remove from the delivery tube, so the removeable plug may be encased in a heat shrink tubing (e.g., tubing that is heat shrunk onto the removable plug and that extends beyond the removable plug away from the delivery tube to provide a length of tubing to grasp) to make it easier to remove. The removable plug may be in the form of a rod (e.g., a long rod) and the rod may be made of an elastomeric polymer. The material of the plug, e.g., the polymer of the rod, can be compatible with components of the drug-containing fluid, such as water, oil, and surfactant. In some embodiments, the removable plug is an elastomeric fluoropolymer rod, such as a Viton™ rod. Additional suitable materials for the removable plug include polymeric elastomers such as perfluoroelastomers (FFKM), fluoroelastomers (FKM and FEPM), polybutadiene (BR), polychloroprene (CR), styrene-butadiene copolymer (SBR), nitrile rubber (NBR), hydrogenated nitrile rubber, (HNBR), ethylene propylene rubber (EPM), ethylene propylene diene rubber (EPDM), chlorosulfonated polyethylene (CSM), and ethylene vinyl acetate rubber (EVA). The elastomeric polymer can optionally be filled with a non-toxic filler such as carbon black or amorphous (non-crystalline) silica. In embodiments in which the removable plug is encased by a heat shrink tubing, the tubing may be made of a heat shrinkable, biocompatible (e.g., compatible with living tissue, for example, the material does not cause sensitivity or irritation of the oral mucosa and does not cause systemic toxicity or cytotoxicity), and chemically inert material, such as fluorinated ethylene propylene (FEP). Both the removable plug (e.g., the elastomeric polymer rod) and part or all of the delivery tube (e.g., part of the delivery tube containing the removable plug or the entire length of the delivery tube) can be sheathed with a polymer sheath. In embodiments in which the removable plug is encased by a heat shrink tubing, the polymer sheath also covers the removable plug/heat shrink tubing assembly. The polymer sheath can serve to slow water permeation (e.g., water permeation through the wall of the delivery tube), preventing the drug-containing fluid in the drug delivery tube from drying. In addition, the polymer sheath can slow oxygen permeation (e.g., oxygen permeation through the wall of the delivery tube). The polymer sheath can also be made of a heat shrinkable, biocompatible, and chemically inert material, and can provide hydrophobicity and low oxygen permeability. The polymer sheath (e.g., the water permeation-slowing polymer sheath) can be a fluoropolymer sheath, such as a sheath of fluorinated ethylene propylene (FEP), which helps reduce water loss through the wall of the delivery tube. Due to its low water permeation rate, an FEP sheath can prevent or reduce the drying of the drug-containing fluid in the delivery tube during extended storage. The polymer sheath (e.g., the FEP sheath) can be heat shrunk over the removable plug (e.g., the elastomeric polymer rod, including the removable plug/heat shrink tubing assembly if the removable plug is encased by a heat shrink tubing) and part or all of the delivery tube and can extend past the plug (e.g., past the plug or past the removable plug/heat shrink tubing assembly if the removable plug is encased by a heat shrink tubing) in the delivery tube, thereby maintaining a mechanical seal between the delivery tube and the removable plug. The polymer sheath can extend beyond the removable plug (e.g., the elastomeric polymer rod or past the removable plug/heat shrink tubing assembly if the removable plug is encased by a heat shrink tubing) in both directions, meaning that the tail of the sheathing can extend beyond the removable plug (or the removable plug/heat shrink tubing assembly) in the direction opposite the delivery tube and the head of the sheathing can extend beyond the removable plug (or the removable plug/heat shrink tubing assembly) to cover all or a portion of the delivery tube into which the plug does not extend. In some embodiments, the tail of the sheathing extends beyond the removable plug (or the removable plug/heat shrink tubing assembly). In some embodiments, both the head and tail of the sheathing extend beyond the removable plug (or the removable plug/heat shrink tubing assembly). In some embodiments, the polymer sheath covers the entire length of the delivery tube and the entire removable plug (or the removable plug/heat shrink tubing assembly, e.g., to provide hydrophobicity and low oxygen permeability across the entire length of the delivery tube). In some embodiments, the polymer sheath covers the entire length of the delivery tube and the entire removable plug and extends beyond the removable plug in the direction opposite the delivery tube (e.g., the tail of the polymer sheath extends beyond the removable plug or the removable plug/heat shrink tubing assembly to apply additional retention force to maintain the removable plug inside the delivery tube). The tail of the polymer sheath can contain a slit and the sheath can be peeled off (e.g., removed from the removable plug and delivery tube) by pulling apart its slit tail. After the sheath is peeled off, part of the removable plug or the removable plug/heat shrink tubing assembly is exposed (e.g., the part of the removable plug or the removable plug/heat shrink tubing assembly that extends beyond the end of the delivery tube). The removable plug can then be pulled out of the delivery tube (e.g., by pulling on the heat shrink tubing) to start the flow of the drug-containing fluid. Exemplary dimensions for the polymer sheath include a length of about 105 mm, an internal diameter of about 0.114 inches, and a thickness of approximately 0.008 inches.

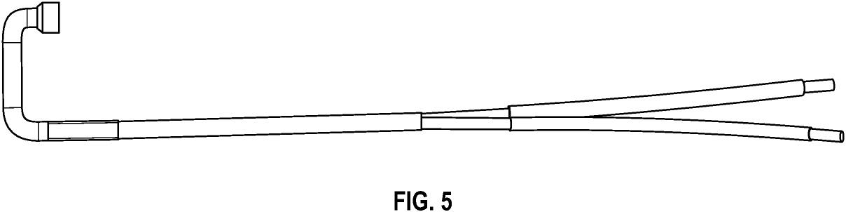

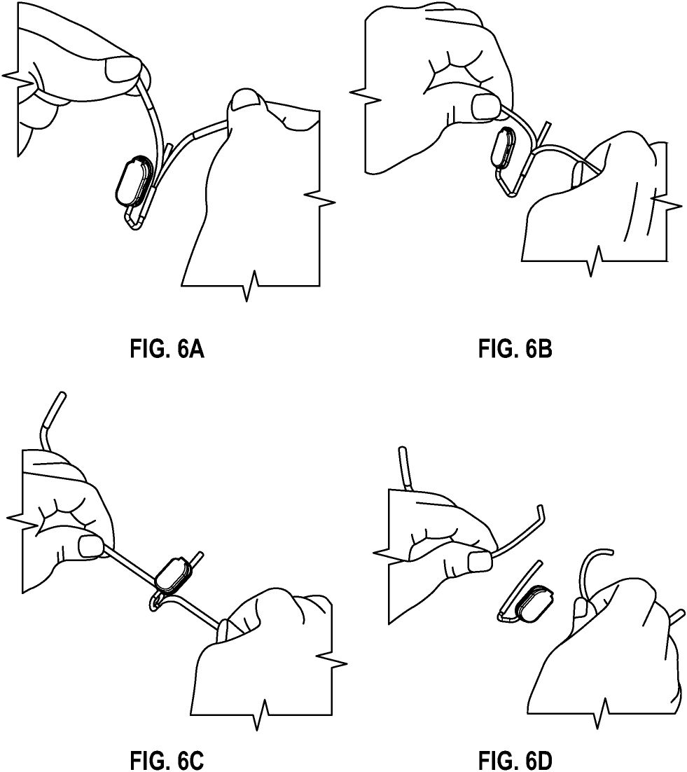

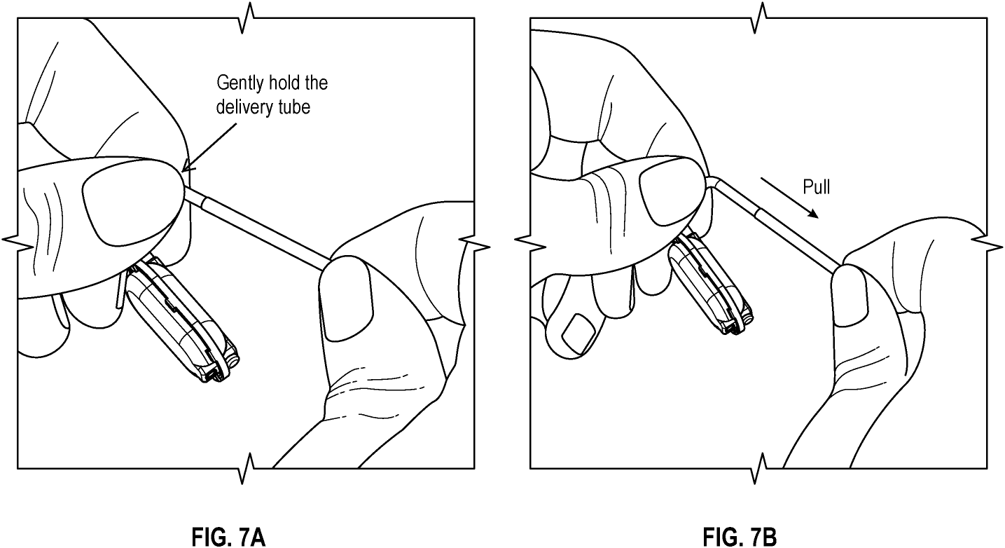

Removable plugs are exemplified in FIGS. 1, 3A-3B, 5, 6A-6B, and 7A-7B. In the drug delivery device of FIG. 1, removable plug 12 can be inserted into delivery tube 9 and 10 to seal the delivery tube and to stop or prevent the flow of drug-containing fluid (e.g., before use). Removable plug 12 can be encased by heat shrink tubing 13. The removable plug, heat shrink tubing, and all or part of the delivery tube can be sheathed with a heat-shrunk polymer sheath, such as an FEP sheath. Removable plug 12 and delivery tube 9 and 10 of FIG. 1 can be sheathed with polymer sheath, which slows water vapor permeation, thereby preventing drying of the drug-containing fluid in delivery tube 9 and 10. For example, FIG. 1 shows an embodiment in which the entire length of delivery tube 9 and 10, removable plug 12, and heat shrink tubing 13 are sheathed by polymer sheath 11. A cross-sectional view of part of an exemplary stored assembly is shown in FIG. 5. FIG. 5 shows a removable plug inserted into a delivery tube, encased in a heat shrink tubing, and enwrapped with a polymer sheath extending beyond the removable plug. The slit tail of the sheath is shown on the right of FIG. 5. The slit tail of the sheath can be pulled apart, as shown in FIGS. 6A-6D, to facilitate peeling off the sheath so the removable plug can be accessed and removed before the device is inserted into the mouth of a patient. In FIGS. 6A-6D, the removable plug is encased in a heat shrink tubing and the polymer sheath enwraps the removable plug, heat shrink tubing, and the entire length of the delivery tube. The entirety of the polymer sheath is removed. FIGS. 7A-7B demonstrate how the removable plug encased in the heat shrink tubing can be removed from the delivery tube to start the flow of the drug-containing fluid.

A removable plug, heat shrink tubing, and/or sheath can be used with any drug delivery device containing a multilayered, reversibly compressible delivery tube described herein, regardless of the type of pump included in the device, including pumps described in U.S. Publication Nos. US2016-0278899A1 and US2017-0172961A1, which are incorporated herein in their entirety by reference.

### Pausing Extrusion of the Drug-Containing Fluid During Use of the Drug Delivery Device

A patient using a drug delivery device described herein may wish to remove the device (e.g., the fastener-mounted drug delivery device) from the mouth during use, such as during meals or toothbrushing. When the device is removed from the mouth, extrusion of the drug-containing fluid can be temporarily paused. If the extrusion of the drug-containing fluid is not paused, the drug-containing fluid may continue to be extruded on the fastener (e.g., the retainer), leading to confusion about the actual dose and the potential for delivery of a bolus when the fastener (covered in the extruded paste) is reinserted in the mouth.

In one embodiment, extrusion of the drug-containing fluid can be paused using a storage case. The storage case can be used to store the drug delivery device when it is separated from the fastener or it can be used to store the drug delivery device while it is attached to or mounted on the fastener (e.g., a retainer). The storage case is constructed such that a component of the case pinches or kinks the delivery tube of the drug delivery device when the storage case is closed, thereby stopping extrusion of the drug-containing fluid. Accordingly, placing the drug delivery device, such as a fastener-mounted (e.g., retainer-mounted) drug delivery device, in the storage case and closing the case (e.g., shutting the lid of the case) allows the patient to pause extrusion of the drug-containing fluid. Closing the storage case stops the extrusion of the drug-containing fluid, and opening the case (e.g., lifting the lid of the storage case), causes the flow of the drug-containing fluid to resume. Opposing components in the case (e.g., components on opposing interior surfaces of the case) that meet (e.g., come together) when the case is closed can be used to pinch or kink the drug delivery tube. An exemplary mechanism for pinching or kinking the delivery tube when the storage case is closed includes a pin (e.g., a metal pin) and an elastomeric pad. The delivery tube can be positioned on the elastomeric pad and the metal pin can compress the delivery tube on the elastomeric pad when the storage case is closed, indenting the elastomeric pad while the tube is pinched or kinked.

The storage case can be made of materials that can be cleaned by wiping and rinsing with water and, optionally, of materials that prevent, or do not support, microbial growth. The patient, or the caregiver, can be provided with instructions for cleaning or sterilizing the case. Optionally, the case can include an ultraviolet light source for reducing or eliminating microbial growth, such as an ultraviolet light source emitting at wavelengths equal to or shorter than 320 nm, such as between 260 nm and 320 nm, or between 275 nm and 290 nm. The light source can include one or more light emitting diodes and the case can include a battery, such as a lithium-containing battery, to power the light source. Optionally, the surface of the case (e.g., an interior surface of the case) can contain silver particles or a silver compound to extend bactericidal activity to longer wavelengths, such as wavelengths in the range between 300 nm and 450 nm, such as 400-450 nm, 350-400 nm, or 300-400 nm.

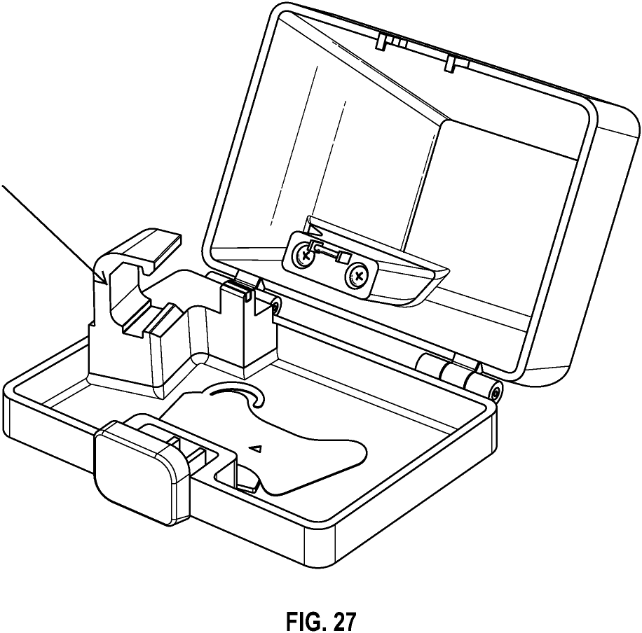

The storage case can also include one or more components to align the drug delivery device and/or fastener (e.g., a fastener, such as a retainer, to which the drug delivery device is mounted) within the case, and can be configured to accommodate a drug delivery device mounted on the right or left side of a patient’s retainer. An exemplary component that can be used to align the drug delivery device is the holder indicated by an arrow in the storage case shown in FIG. 27. The holder can be used to align the drug delivery device when it is mounted on a retainer to properly position the delivery tube such that it will be pinched or kinked with the storage case is closed.

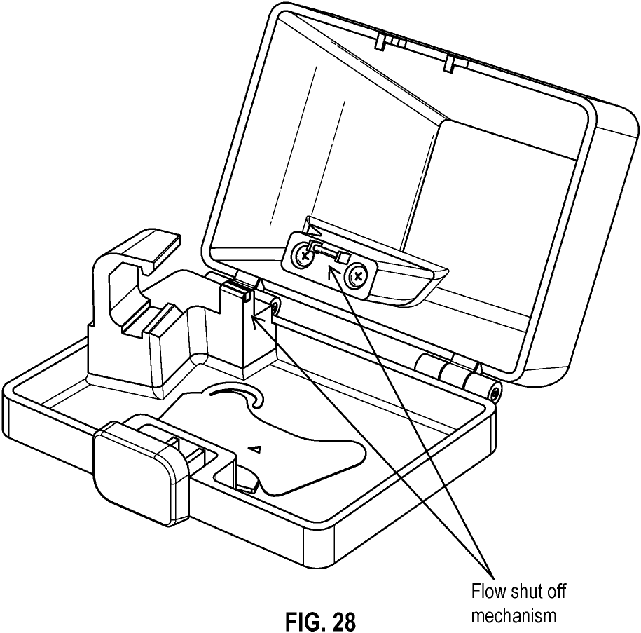

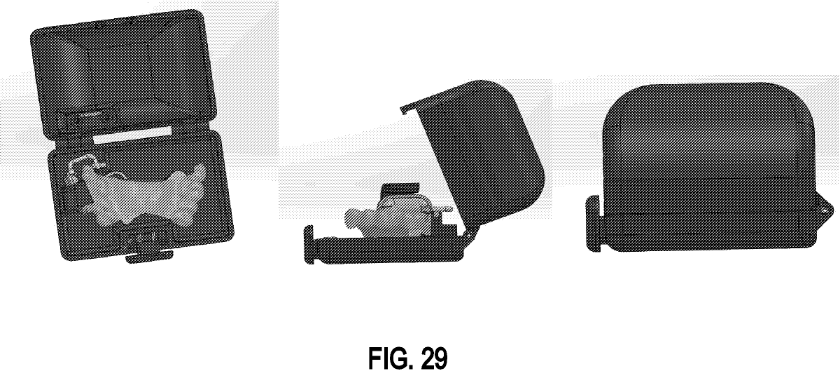

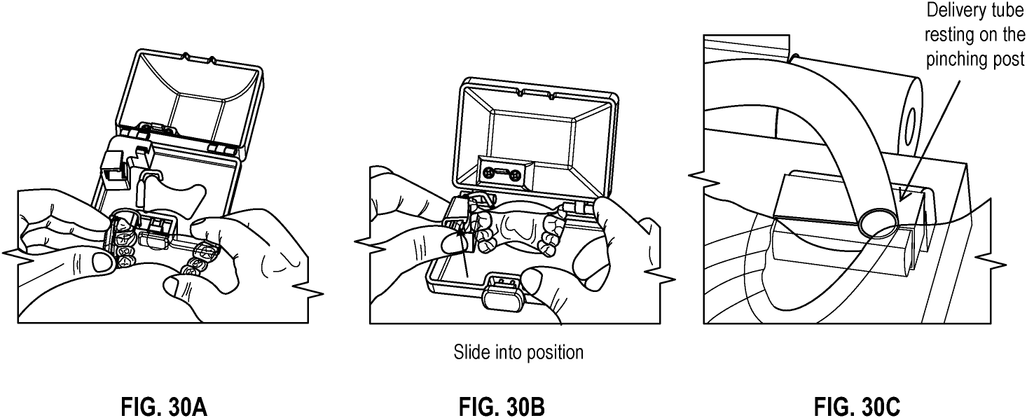

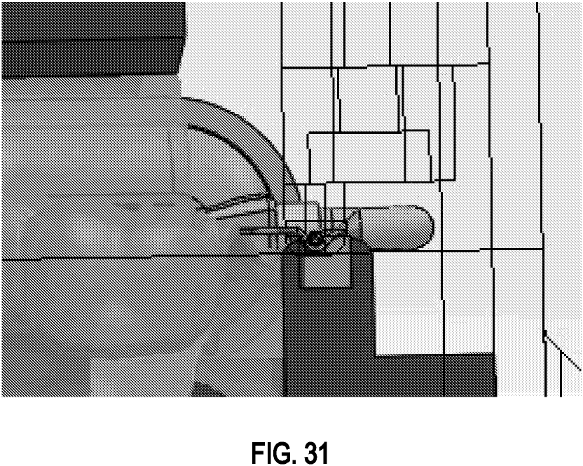

An exemplary storage case is shown in FIGS. 27 and 28. The case is configured to hold a retainer with a drug delivery device (e.g., a retainer-mounted drug delivery device) such that, when the case is closed, the delivery tube of the drug delivery device is pinched, stopping extrusion of the drug-containing fluid from the device. Upon removal from the case, the delivery tube is no longer pinched and extrusion of the drug-containing fluid resumes. The components on opposing interior surfaces of the storage case that pinch or kink the delivery tube when the storage case is closed are indicated using arrows in FIG. 28 (described as a “flow shut off mechanism”). The storage case also contains a picture of the drug device on the bottom interior surface of the case to direct a patient or caregiver to insert the drug delivery device in the correct orientation. FIG. 29 exemplifies the positions of the retainer and the drug delivery device in the storage case and FIGS. 30A-30C illustrate how the drug delivery device is inserted into the storage case, including how the drug delivery device is inserted into the holder. FIG. 31 provides a schematic of the kinking or pinching of the delivery tube when the case is shut. Once the drug delivery device is inserted into the storage container and the case is closed to pause drug extrusion, it can be stored for up to 5 hours at room temperature.

A storage case that can be used to temporarily stop extrusion of a drug-containing fluid can be used with any drug delivery device containing a multilayered, reversibly compressible delivery tube described herein, regardless of the type of pump included in the device, including pumps described in U.S. Publication Nos. US2016-0278899A1 and US2017-0172961A1, which are incorporated herein in their entirety by reference.

### Packaging for Long-term Storage of the Drug Delivery Device

A drug delivery device described herein can be stored for at least 6 months at a temperature between about 10° C. and about -2° C., such as between 5° C. and 8° C., in a hermetically sealed pouch with an atmosphere that is substantially free of oxygen, such as a nitrogen, argon, or carbon dioxide atmosphere. The pouch wall can contain a laminate of a metal film and a polymer, such as a metallized polymer. The packaging can be used to minimize oxygen diffusion into the device. Optionally, the sealed pouch can contain a humidifying porous insert, such as porous paper or gauze, the insert wetted with water, or with an aqueous solution, the solution having at a particular temperature a water vapor pressure lower than that of pure water, so as to maintain in the pouch a relative humidity near, but less than, 100%.

### DRUG FORMULATIONS

Formulations of drugs to be delivered via the drug delivery devices (such as LD, LD prodrugs, DDC inhibitors, and any other drugs described herein) may include non-toxic aqueous or non-aqueous carrier liquids, such as water, and edible oils such as vegetable oils, lipids, triglycerides, paraffin oil, and their mixtures. Formulations that can be delivered include those described in U.S. Pat. Publication No. US2017-0172961A1, which is incorporated by reference herein in its entirety.

### EXAMPLES

The following examples are meant to illustrate the invention. They are not meant to limit the invention in any way.

### Example 1 Study of Continuous Oral Levodopa

The DopaFuse Delivery System is a novel, intra-oral system that continuously delivers LD/CD at a controlled rate for patients with PD. The DopaFuse System consists of an oral retainer, its case, and a single-use drug delivery device that continuously releases an investigational LD/CD paste in the back of the mouth.

The DopaFuse Delivery System was developed to provide the benefits of continuously delivered LD/CD without the need for a surgical procedure (e.g., with duodenum infusion) and the associated adverse effects. DopaFuse provides continuous oral levodopa via a container, secured by a retainer worn on the teeth, which releases a LD/CD paste at a constant rate. A proof of concept study of continuous oral levodopa therapy (using sips of an oral LD/CD suspension at 5-10 minute intervals) demonstrated that continuous oral levodopa therapy was well tolerated and significantly reduced plasma levodopa variability and Off time in comparison to standard oral LD/CD tablets. As a next step, this study is designed to evaluate the pharmacokinetics, safety, efficacy and tolerability of the system.

The purpose of this study is to evaluate whether the DopaFuse System can reduce the fluctuation of plasma levodopa levels as compared to participants’ standard intermittent doses of oral LD/CD tablets (background treatment). An assessment of whether the system is safe, well tolerated, and can relieve motor symptoms will also be made.

### Participants

Approximately 30-35 participants will be screened to achieve an estimated total of 24 participants. The patients have Parkinson’s Disease and are on a stable LD/CD treatment and experience at least 2 hours of OFF time per day. Patients will be asked to stop taking COMT inhibitors.

### Dosing

DopaFuse is an oral Paste containing suspended particles of micronized levodopa and carbidopa. Two infusion rates: (i) 50 mg LD/13 mg CD per hour; and (ii) 68 mg LD/17 mg CD per hour will be administered. Treatment regimens will be individualized in order to closely align with the participant’s usual dosing quantities and timetable. Over the 15-day active study period, each participant will receive DopaFuse treatment for three days under inpatient observation and 11 days via in-home use. On Day 1, participants will take their usual oral immediate release LD/CD regimen. On Day 2, participants will receive DopaFuse alone. Days 3-15 will involve participants taking their usual initial AM dosing of LD/CD, followed by continuous DopaFuse for the rest of the day. A study summary is shown in Table 5.

TABLE 5

Study Summary.

| | |
|---|---|
| Screening & Retainer Manufacturing | •Screening Activates |
| | •Dental exam |
| | •Retainer manufacturing and fitting |
| Day 1-Baseline Oral LD/CD Tablets^{∗} (Inpatient) | •Participant’s usual oral LD/CD regimen |
| | •PK measurements |
| | •Motor assessments |
| Day 2: DopaFuse Alone (Inpatient) | •DopaFuse alone |
| | •No oral LD/CD tablets |
| | •PK measurements |
| | •Motor assessments |
| Day 3: DopaFuse + Oral LD/CD Tablets^{∗} (Inpatient) | •Participant’s usual oral LD/CD morning dose |
| | •DopaFuse for the rest of the day |
| | •PK measurements |
| | •Motor measurements |
| Days 4-14: DopaFuse + Oral LD/CD Tablets^{∗} (Home Use) | •Participant’s usual oral LD/CD morning dose |
| | •DopaFuse for the rest of the day |
| Day 15: DopaFuse + Oral LD/CD Tablets^{∗} (Inpatient) | •Participant’s usual oral LD/CD morning dose |
| | •DopaFuse for the rest of the day |
| | •Motor assessments |
| Day 29: Safety & Follow Up | •Final Safety Televisit |

*Oral LD/CD tablets refers to participants’ previously prescribed oral LD/CD.

### DopaFuse Delivery System

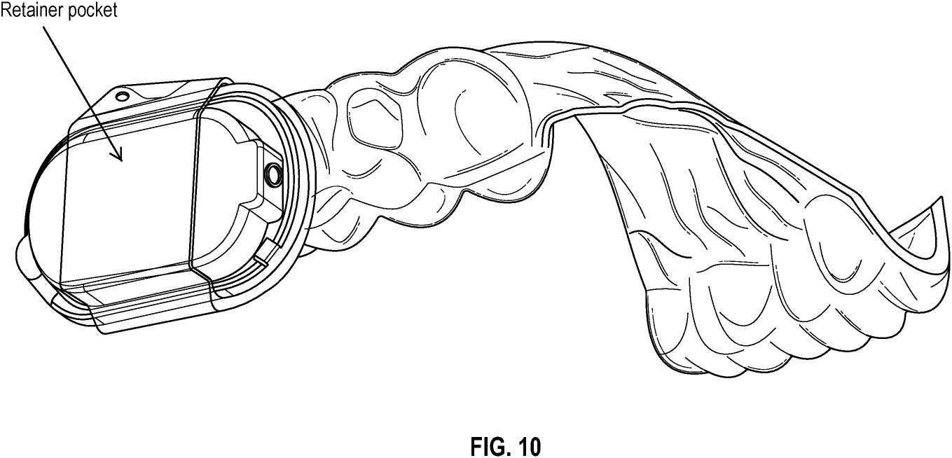

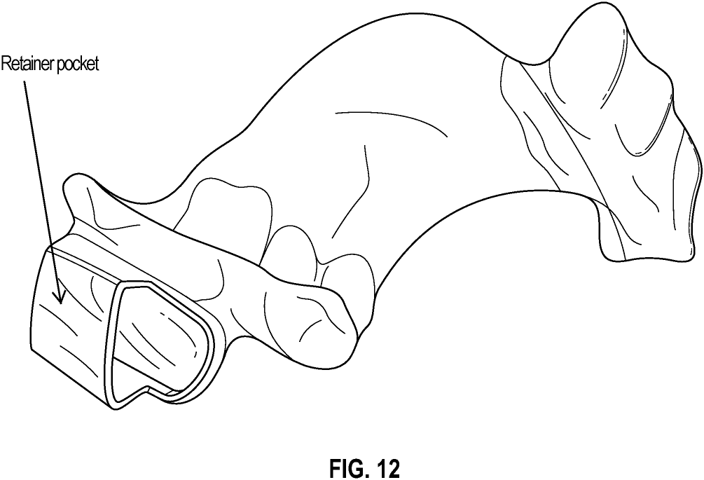

Participants will be given a reusable custom dental retainer, its storage case, and a single-use drug delivery device prefilled with the LD/CD paste described above (LD:CD in a ratio of 4:1). The retainer is a re-usable, custom-made thermoformed retainer made of a dental thermoform material called Essix® PLUS™. The retainer has a pocket molded on one side to hold the single-use drug delivery device and positions the drug delivery device on the buccal side of the upper molars coplanar with the occlusal plane of the first or second molar. The retainer is worn over the bicuspids and molars, but the drug delivery device will span only the first and second molars and will be positioned in the pocket such that the delivery tube of the drug delivery device wraps around the rear-most molar of the participant. The retainer is shown in FIGS. 10 and 12. FIGS. 13A-13C and 14 show how the container can be inserted into and removed from the retainer pocket and how the retainer containing the drug delivery device is inserted into the mouth.

The drug delivery device is a disposable, pre-filled single-use container constructed as shown in FIGS. 9 and 21. The drug delivery device includes a propellant chamber and a drug chamber separated by a deformable metallic diaphragm. The propellant chamber and drug chamber have titanium housings and are hermetically sealed using a two-component adhesive epoxy (LOCTITE®). The adhesive bonds are strengthened by the step included on the rim of each housing that increases the adhesive-bonded area (see FIGS. 17-19). The drug delivery device is further strengthened through the use of crimpable tabs in the housings (see FIGS. 20 and 21). The LD/CD paste leaves the drug delivery device through two flow restrictors of different lengths as described in Table 3 before it is extruded into a reversibly compressible, multilayered delivery tube. The delivery tube contains two laminated polymer layers, an outer polyurethane layer and an inner PET layer, and can revert to about its original shape after a patient bites on it or after it is pinched or kinked. If the delivery tube is bitten, pinched, kinked, or otherwise compressed, the rate of drug delivery changes by less than 10% after the biting, pinching, kinking, or compression ends. The propellant chamber is hermetically sealed using a nitrile rubber septum and the drug chamber is hermetically sealed using an elastomeric Viton™ plug. Prior to use, the delivery tube is hermetically sealed using a removable Viton™ plug, the removable Viton™ plug is encased in an FEP heat shrink tubing, and the removable plug, heat shrink tubing, and drug delivery tube are enwrapped by an FEP sheath. The removable plug prevents the LD/CD paste from being extruded before the drug delivery device is used, the FEP heat shrink tubing makes the removable plug easier to remove from the delivery tube before the device is inserted into the mouth of a patient, and FEP sheath can prevent or reduce the drying of the LD/CD paste in the delivery tube during extended storage and maintain a mechanical seal between the delivery tube and the removable plug. The FEP sheath can be peeled off by pulling apart its slit tail and the removable plug can be pulled out of the delivery tube using the heat shrink tubing to start the flow of the LD/CD paste, as shown in FIGS. 6A-6D and 7A-7B. The drug delivery device can then be pushed into the pocket of a retainer and the retainer can be inserted into the mouth for use (see FIGS. 8-10, 13A-13B, and 14A-14B). The drug delivery devices are packaged in a non-sterile plastic tray sealed in a foil pouch with an atmosphere that is substantially free of oxygen that must be refrigerated (2- 8° C.) during storage. The composition of the LD/CD paste that is contained in the drug delivery device is shown below in Table 5.

TABLE 5

Composition of LD/CD paste

| Ingredient | Quality Standard | Function | Quantity | |
|---|---|---|---|---|
| | | | mg/container | % w/w |
| Levodopa | Ph. Eur. | Active | 495.0 | 49.5 |
| Carbidopa | Ph. Eur. | Active | 133.7 | 13.4 |
| Poloxamer 188 | Ph. Eur. | Emulsifier | 49.5 | 5.0 |
| Medium Chain Triglycerides (Miglyol 812) | Ph. Eur. | Suspending liquid | 252.5 | 25.3 |
| Purified Water | Ph. Eur. | Suspending liquid | 65.3 | 6.5 |
| Benzoic Acid | Ph. Eur. | Antimicrobial | 3.0 | 0.3 |
| Sodium Edetate (EDTA) | Ph. Eur. | Antioxidant | 1.0 | 0.1 |
| Total | | | 1000.0 | 100.0 |

Participants are also provided with a storage case for the retainer and drug delivery device. The DopaFuse case functions to physically protect the retainer during storage and contains a mechanism to pause the flow of LD/CD paste from the drug delivery device when the retainer-mounted drug delivery device is placed into the case and the case is closed. When the storage case is opened and the retainer/drug-container are removed, flow of the paste resumes. Participants can use the storage case to temporarily stop extrusion of the LD/CD paste through the delivery tube when the retainer/drug-container are removed from the mouth, such as during meals or for toothbrushing. The mechanism for pausing the flow of the LD/CD paste includes a metal pin and an elastomeric pad. The delivery tube can be positioned on the elastomeric pad and the metal pin can compress the delivery tube on the elastomeric pad when the storage case is closed, thereby pinching the delivery tube and stopping the flow of the paste. The storage case also contains a picture of the drug device on the bottom interior surface of the case to direct a participant or caregiver to insert the drug delivery device in the correct orientation and contains a holder to correctly align the retainer-mounted drug delivery device. The storage case is made of materials that can be cleaned by wiping and rinsing with water and that do not support microbial growth. FIGS. 27-31 show images of the storage container and its use.

### Pharmacokinetic Analyses

he primary estimand for the PK study will be the difference in levodopa fluctuation index (Fl, Cmax-Cmin)/Caverage) between Day 1 (oral levodopa) and Day 2 (DopaFuse alone). As steady-state levodopa concentrations are not anticipated to be achieved during the first 4 hours of treatment with continuous oral administration, the fluctuation index will be evaluated between hours 4 and 12. Statistical analyses will be performed using the 2-tailed t-test for continuous data and Wilcoxon signed rank test for non-continuous data. Sensitivity analyses will include fluctuation indices in the Per Protocol population. The fluctuation index will also be assessed at hourly intervals for additional sensitivity analyses.

### Results

This study can demonstrate the importance of using a retainer-mounted drug delivery device for continuous or semi-continuous intraoral infusion of a drug on PK performance. It is believed that superior results can be achieved using the retainer-mounted drug delivery device to achieve continuous or semi-continuous intraoral infusion of a drug compared to administration of the same drug in at least four doses per day.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising:
(a) a drug reservoir comprising said pharmaceutical composition, the volume of said drug reservoir being from 0.1 mL to 5 mL; and
(b) a reversibly compressible, multi-layered delivery tube in fluidic communication with the drug reservoir for delivery of the pharmaceutical composition to the mouth of the patient that is configured to recover its shape after compression such that the steady-state rate of administration of said pharmaceutical composition from said drug delivery device increases or decreases by less than 10% after said compression is released.

2. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth;
(ii) a pump comprising:
(a) a drug reservoir comprising said pharmaceutical composition, the volume of said drug reservoir being from 0.1 mL to 5 mL; and
(b) a delivery tube in fluidic communication with the drug reservoir for delivery of the pharmaceutical composition to the mouth of the patient; and
(iii) a removable plug comprising an elastomeric polymer for hermetically sealing the delivery tube prior to initial use by the patient, wherein, following removal of the removable plug, the device is ready to be inserted into the mouth of the patient for said intraoral administration.

3. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising a drug reservoir comprising said pharmaceutical composition, the volume of said drug reservoir being from 0.1 mL to 5 mL;
wherein the fastener comprises a pocket that is fabricated as an integral part of the fastener for securing the drug delivery device to a buccal side of an upper molar of the patient, and wherein the pocket is configured to position the drug delivery device coplanar with an occlusal plane of the molar.

4. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising a drug reservoir comprising said pharmaceutical composition, the volume of said drug reservoir being from 0.1 mL to 5 mL,
wherein the drug reservoir comprises a rigid housing having a port for filling the drug reservoir with said pharmaceutical composition that is hermetically sealed with an elastomeric plug comprising a fluorocarbon elastomer that is biocompatible with the oral mucosa and compatible with the pharmaceutical composition.

5. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising a drug reservoir comprising said pharmaceutical composition, the volume of said drug reservoir being from 0.1 mL to 5 mL;
wherein the drug delivery device comprises two flow restrictors fluidically connected to the drug reservoir, and wherein a first flow restrictor has a length of 6 - 12 mm and an internal diameter of 0.0100 0.0120 inches, and a second flow restrictor has a length of 1424 mm and an internal diameter of 0.0130 - 0.0145 inches.

6. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising:
(a) a drug reservoir comprising said pharmaceutical composition, the volume of said drug reservoir being from 0.1 mL to 5 mL;
(b) a flow restrictor in fluidic communication with the drug reservoir; and
(c) a reversibly compressible delivery tube in fluidic communication with the drug reservoir for delivery of the pharmaceutical composition to the mouth of the patient,
wherein said flow restrictor and/or said delivery tube are hermetically bonded either directly or indirectly to the drug reservoir by an adhesive.

7. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a propellant-driven pump comprising a drug reservoir comprising said pharmaceutical composition, the volume of said drug reservoir being from 0.1 mL to 5 mL;
wherein the propellant-driven pump comprises a rigid housing, an eyelet, and a compressible, elastomeric septum for propellant loading fitted into the eyelet to form a hermetic seal.

8. A drug delivery device configured to be removably inserted in a patient’s mouth and for continuous or semi-continuous intraoral administration of a pharmaceutical composition comprising a drug, said device comprising:
(i) a first chamber comprising the pharmaceutical composition;
(ii) a second chamber comprising a propellant; and
(iii) a flexible and/or deformable diaphragm separating said first chamber from said second chamber,
wherein said first chamber, said second chamber, and, optionally, said diaphragm are bonded by an adhesive to form a hermetic seal.

9. A drug delivery device configured to be removably inserted in a patient’s mouth and for continuous or semi-continuous intraoral administration of a pharmaceutical composition comprising a drug, said device comprising:
(i) a first chamber comprising the pharmaceutical composition;
(ii) a second chamber comprising a propellant; and
(iii) a flexible and/or deformable diaphragm separating said first chamber from said second chamber,
wherein said first chamber, said second chamber, and, optionally, said diaphragm are sealed by a crimp.

10. A drug delivery device configured to be removably inserted in a patient’s mouth and for continuous or semi-continuous intraoral administration of a pharmaceutical composition comprising a drug, said device comprising:
(i) a first chamber comprising the pharmaceutical composition;
(ii) a second chamber comprising a propellant; and
(iii) a flexible and/or deformable diaphragm separating said first chamber from said second chamber,
wherein said first chamber comprises a first rigid housing, said second chamber comprises a second rigid housing, and wherein the first chamber and the second chamber each comprise a rim having a step to increase the adhesive-bonded area.

11. A drug delivery device configured to be removably inserted in a patient’s mouth and for continuous or semi-continuous intraoral administration of a pharmaceutical composition comprising a drug, said device comprising:
(i) a fastener to removably secure said drug delivery device to a surface of said patient’s mouth; and
(ii) a pump comprising a drug reservoir comprising said pharmaceutical composition, the volume of said drug reservoir being from 0.1 mL to 5 mL;
wherein the fastener is configured such that the patient’s oral anatomy would mechanically interfere with and prevent the removal of the drug delivery device from the fastener when the fastener is attached to the surface of the patient’s mouth.

12. The drug delivery device of claim 2, wherein the delivery tube is a reversibly compressible, multi-layered delivery tube that is configured to recover its shape after compression such that the steady-state rate of administration of said pharmaceutical composition from said drug delivery device increases or decreases by less than 10% after said compression is released.

13. The drug delivery device of any one of claims 3-11, wherein the device further comprises a reversibly compressible, multi-layered delivery tube in fluidic communication with the drug reservoir or first chamber for delivery of the pharmaceutical composition to the mouth of the patient that is configured to recover its shape after compression such that the steady-state rate of administration of said pharmaceutical composition from said drug delivery device increases or decreases by less than 10% after said compression is released.

14. The drug delivery device of any one of claims 1, 12, and 13, wherein administration of the pharmaceutical composition can be temporarily stopped when the drug delivery device is in use by compressing the delivery tube.

15. The drug delivery device of any one of claims 1 and 12-14, wherein the delivery tube comprises an outer layer and an inner layer comprising laminated polymers, and wherein the outer layer comprises a polymer that is biocompatible with the oral mucosa.

16. The drug delivery device of claim 15, wherein the outer layer comprises polyurethane, poly(ethylene glycol) diacrylate, or poly(lactide-co-glycolide) (PLGA).

17. The drug delivery device of claim 16, wherein the outer layer comprises polyurethane.

18. The drug delivery device of any one of claims 15-17, wherein the thickness of the outer layer is between 0.05 mm and 0.2 mm.

19. The drug delivery device of any one of claims 15-18, wherein the inner layer comprises a polymer that is compatible with the pharmaceutical composition and that provides radial strength and reduces water permeation through the delivery tube.

20. The drug delivery device of any one of claims 15-19, wherein the inner layer comprises polyethylene terephthalate (PET), high density polyethylene (HDPE), polyvinylidene chloride (PVDC), polytetrafluoroethylene (PTFE), or polymeric fluorinated ethylene propylene (FEP).

21. The drug delivery device of claim 20, wherein the inner layer comprises PET.

22. The drug delivery device of any one of claims 15-21, wherein the thickness of the inner layer is between 0.025 mm and 0.075 mm.

23. The drug delivery device of any one of claims 1 and 12-22, wherein the delivery tube substantially re-acquires its internal diameter after compression.

24. The drug delivery device of any one of claims 1 and 12-23, wherein the drug delivery device further comprises a removable plug comprising an elastomeric polymer for hermetically sealing the delivery tube prior to initial use by the patient, and wherein, following removal of the removable plug, the device is ready to be inserted into the mouth of the patient for said intraoral administration.

25. The drug delivery device of claim 2 or 24, wherein the removable plug prevents extrusion of the pharmaceutical composition from the drug delivery device.

26. The drug delivery device of any one of claims 2, 24, and 25, wherein the elastomeric polymer is compatible with the pharmaceutical composition.

27. The drug delivery device of any one of claims 2 and 24-26, wherein the removable plug comprises a fluoropolymer.

28. The drug delivery device of any one of claims 2 and 24-27, wherein the removeable plug is encased in a heat shrink tubing to facilitate its removal from the drug delivery device.

29. The drug delivery device of claim 28, wherein the heat shrink tubing comprises a fluoropolymer.

30. The drug delivery device of any one of claims 2 and 24-27, wherein the removeable plug and all or a portion of the delivery tube are enwrapped in a polymeric sheath prior to initial use of the drug delivery device by the patient.

31. The drug delivery device of any one of claims 28-30, wherein the heat shrink tubing is enwrapped in a polymeric sheath prior to initial use of the drug delivery device by the patient.

32. The drug delivery device of claim 30 or 31, wherein the polymeric sheath comprises a fluoropolymer.

33. The drug delivery device of any one of claims 30-32, wherein the polymeric sheath slows water vapor permeation, thereby reducing or preventing drying of pharmaceutical composition in the delivery tube.

34. The drug delivery device of any one of claims 30-33, wherein the delivery tube is water impermeable when enwrapped by the polymeric sheath such that the pharmaceutical composition does not dry in the delivery tube.

35. The drug delivery device of any one of claims 30-34, wherein the polymeric sheath comprises a slit tail.

36. The drug delivery device of any one of claims 30-35, wherein the polymeric sheath is configured to be removed before insertion of the drug delivery device into the mouth of the patient.

37. The drug delivery device of any one of claims 2 and 24-36, wherein the removable plug is configured to be removed before insertion of the drug delivery device into the mouth of the patient.

38. The drug delivery device of any one of claims 8-10, wherein the drug delivery device further comprises a fastener to removably secure said drug delivery device to a surface of said patient’s mouth.

39. The drug delivery device of any one of claims 1, 2, 4-7, and 11-38, wherein the fastener comprises a pocket that is fabricated as an integral part of the fastener for securing the drug delivery device to a buccal side of an upper molar of the patient, and wherein the pocket is configured to position the drug delivery device coplanar with an occlusal plane of the molar.

40. The drug delivery device of claim 3 or 39, wherein the pocket is configured to position the drug delivery device coplanar with the occlusal plane of the first or second molar.

41. The drug delivery device of any one of claims 3, 39, and 40, wherein the pocket is configured to position the drug delivery device such that it is set on the Wilson plane.

42. The drug delivery device of any one of claims 1-7 and 11-41, wherein the fastener is worn over the bicuspids and molars.

43. The drug delivery device of any one of claims 1-7 and 11-42, wherein the fastener is made of a plastic.

44. The drug delivery device of claim 43, wherein the plastic is an acrylate-containing polymer or a thermoplastic polyolefin.

45. The drug delivery device of any one of claims 3 and 39-44, wherein the pocket is configured to position the drug delivery device such that the pharmaceutical composition is released from a delivery tube on a lingual side of the patient’s teeth.

46. The drug delivery device of any one of claims 3 and 39-45, wherein the pocket is configured such that the patient’s oral anatomy would mechanically interfere with and prevent the removal of the drug delivery device from the pocket when the fastener is attached to the surface of the patient’s mouth.

47. The drug delivery device of any one of claims 3 and 39-46, wherein the pocket is configured such that it has a friction fit with the drug delivery device and thereby prevents the drug delivery device from being dislodged from the pocket during insertion into the mouth or administration of the pharmaceutical composition.

48. The drug delivery device of any one of claims 3 and 39-47, wherein the drug delivery device can be removed from the pocket by pushing on the device.

49. The drug delivery device of any one of claims 1-7 and 11-48, wherein the fastener is a retainer.

50. The drug delivery device of any one of claims 1, 2, 3, and 5-49, wherein the drug reservoir or the first chamber comprises a rigid housing having a port for filling the drug reservoir or chamber with said pharmaceutical composition that is hermetically sealed with an elastomeric plug comprising a fluorocarbon elastomer that is biocompatible with the oral mucosa and compatible with the pharmaceutical composition.

51. The drug delivery device of claim 4 or 50, wherein the elastomeric plug is an injection molded elastomeric plug.

52. The drug delivery device of any one of claims 4, 50, and 51, wherein the port sealed with the elastomeric plug can sustain a pressure of about 12 bar when the housing wall has a thickness of about 0.015 inches.

53. The drug delivery device of any one of claims 1-4 and 6-52, wherein drug delivery device comprises two flow restrictors fluidically connected to the drug reservoir or first chamber, and wherein a first flow restrictor has a length of 6 - 12 mm and an internal diameter of 0.0100 - 0.0120 inches, and a second flow restrictor has a length of 14 - 24 mm and an internal diameter of 0.0130 - 0.0145 inches.

54. The drug delivery device of claim 5 or 53, wherein the two flow restrictors have the dimensions of a configuration listed in Table 3.

55. The drug delivery device of any one of claims 1, 2 and 11-54, wherein the drug delivery device comprises a flow restrictor in fluidic communication with the drug reservoir or first chamber, and wherein said flow restrictor and/or said delivery tube are hermetically bonded either directly or indirectly to the drug reservoir by an adhesive.

56. The drug delivery device of claim 6 or 55, wherein the flow restrictor is bonded to a coupling adaptor, which is then hermetically bonded to the drug reservoir or first chamber.

57. The drug delivery device of any one of claims 6, 55, and 56, wherein the delivery tube is hermetically bonded to the drug reservoir or first chamber.

58. The drug delivery device of any one of claims 1-6 and 11, wherein the pump is a propellant-driven pump.

59. The drug delivery device of any one of claims 8-58, wherein the second chamber or the propellant-driven pump comprises a rigid housing, an eyelet, and a compressible, elastomeric septum for propellant loading fitted into the eyelet to form a hermetic seal.

60. The drug delivery device of claim 7 or 59, wherein the septum is biocompatible with the oral mucosa and compatible with the propellant.

61. The drug delivery device of any one of claims 7, 59, and 60, wherein the septum comprises nitrile rubber.

62. The drug delivery device of any one of claims 7 and 59-61, wherein the septum is flared to prevent its ejection from the device under the pressure in the propellant chamber.

63. The drug delivery device of any one of claims 1-7 and 11-62, wherein said pump is a propellant-driven pump and the device comprises:
(i) a first chamber comprising the pharmaceutical composition;
(ii) a second chamber comprising a propellant; and
(iii) a flexible and/or deformable diaphragm separating said first chamber from said second chamber.

64. The drug delivery device of any one of claims 9, 10, and 63, wherein said first chamber, said second chamber, and, optionally, said diaphragm are bonded by an adhesive to form a hermetic seal.

65. The drug delivery device of any one of claims 8, 63, and 64, wherein the first chamber has a first rigid housing and wherein the second chamber has a second rigid housing, and wherein the first chamber and the second chamber each comprise a rim having a step for increasing the adhesive-bonded area.

66. The drug delivery device of any one of claims 6, 8, and 55-65 wherein the adhesive is acrylate, gum, epoxy, or a sealant.

67. The drug delivery device of claim 66, wherein the epoxy adhesive comprises two components, a first component comprising compound with two or more epoxide functions and a second component comprising a compound with two or more amine functions.

68. The drug delivery device of any one of claims 6, 8, and 55-67, wherein the cured adhesive is compatible with both the propellant and with components of the drug-containing fluid.

69. The drug delivery device of any one of claims 8-10 and 63-68, wherein the first housing and the second housing sandwich the diaphragm.

70. The drug delivery device of any one of claims 8, 10, and 63-69, wherein said first chamber, said second chamber, and, optionally, said diaphragm are sealed by a crimp.

71. The drug delivery device of claim 9 or 70, wherein the first chamber has a first rigid housing and wherein the second chamber has a second rigid housing, wherein the first chamber and the second chamber each comprise a rim having a step, and wherein the step of the rim of either the first housing or the second housing comprises crimped tabs.

72. The drug delivery device of any one of claims 8-10 and 63-71, wherein the second chamber comprises a propellant having a vapor pressure of about 4 atm or greater at 37° C.

73. The drug delivery device of any one of claims 4 and 8-10, and 13-72, wherein each of said rigid housings has a wall thickness between 0.25 mm and 0.5 mm.

74. The drug delivery device of any one of claims 8-10 and 13-73, wherein the diaphragm is 0.025 mm to 0.25 mm thick.

75. The drug delivery device of any one of claims 1, 2, and 12-74, wherein the pharmaceutical composition comprises the drug and one or more excipients, and wherein less than about 3% of said drug and of each of said excipients is absorbed by or transported through said delivery tube when the drug delivery device is stored non-frozen for one year.

76. The drug delivery device of any one of claims 1-75, wherein the pump maintains an internal pressure of greater than or equal to about 2 bar on said pharmaceutical composition.

77. The drug delivery device of any one of claims 1-6, 11-57, 75, and 76, wherein the pump comprises a battery-driven pump, a mechanically-driven pump, or an osmotic pump.

78. A method of manufacturing the drug delivery device of any one of claims 8-77, comprising applying an adhesive to any of said first chamber, said second chamber, said diaphragm, said flow restrictor, and/or said delivery tube, and curing said adhesive to form a hermetic seal.

79. A storage case for temporarily stopping extrusion of a pharmaceutical composition from a drug delivery device, comprising paired components on opposing interior surfaces of the storage case that are configured to pinch or kink a reversibly compressible tube of a drug delivery device when the storage case is closed, thereby temporarily stopping extrusion from the drug delivery device.

80. The storage case of claim 79, wherein the storage case is configured to temporarily stop extrusion from the drug delivery device while the drug delivery device is attached to a fastener configured to removably secure said drug delivery device to a surface of a patient’s mouth.

81. The storage case of claim 79, wherein the storage case is configured to temporarily stop extrusion from the drug delivery device while the drug delivery device is not attached to a fastener configured to removably secure said drug delivery device to a surface of a patient’s mouth.

82. The storage case of any one of claims 79-81, wherein the paired components comprise an elastomeric pad and a metal pin.

83. The storage case of any one of claims 79-82, further comprising a holder to align the drug delivery device in the storage case.

84. The storage case of any one of claims 79-83, wherein the case comprises materials that do not support microbial growth.

85. The storage case of any one of claims 79-84, wherein the drug delivery device is the device of any one of claims 1-75.

86. A method of pausing extrusion of the pharmaceutical composition from the drug delivery device of any one of claims 1-77, comprising inserting the drug delivery device into the storage case of any one of claims 79-85 and closing the lid of the storage case.

87. A method of administering a pharmaceutical composition into a mouth of a patient, the method comprising:
(i) inserting the drug delivery device of any one of claims 1-77 into the mouth of said patient;
(ii) administering said pharmaceutical composition to said patient; and
(iii) removing said device from the mouth of said patient.

88. The method of claim 87, wherein the method further comprises temporarily stopping administration of the pharmaceutical composition by pinching or kinking the delivery tube.

89. The method of claim 88, wherein the tube is pinched or kinked by inserting the drug delivery device into the storage case of any one of claims 79-85 and closing the lid.

90. The method of claim 89, further comprising first inserting the pump for delivery of said pharmaceutical composition into the fastener configured to removably secure said drug delivery device to the surface of the patient’s mouth.

91. A packaging material for long term storage of a drug delivery device, comprising a hermetically sealed pouch that is gas impermeable with an atmosphere that is substantially free of oxygen.
